SEQUENCE LISTING (15 sequences)

SEQ ID NO: 1 - protein, 4 aa, Artificial Sequence
  Synthetic
CAAX

SEQ ID NO: 2 - DNA, 33 bp, Artificial Sequence
  Synthetic
gggggatccatgccggaacagagtaacgattac

SEQ ID NO: 3 - DNA, 31 bp, Artificial Sequence
  Synthetic
gcggaattctcacatgagggtgcatttgccc

SEQ ID NO: 4 - DNA, 597 bp, Homo sapiens
atgccggaacagagtaacgattaccgcgtggtggtgttcggggcgggcggcgtgggcaagagctcgctggtgctgcgcttcgtgaagggcacgttccgcgacacctacatccccaccatcgaggacacctaccggcaggtgatcagctgcgacaagagcgtgtgcacgctgcagatcacagacaccaccggcagccaccagttcccggccatgcagcgcctgtccatctccaagggccacgccttcatcctggtgttctccgtcaccagcaagcagtcgctggaggagctggggcccatctacaagctcatcgtgcagatcaagggcagcgtggaggacatccccgtgatgctcgtgggcaacaagtgcgatgagacgcagcgggaggtggacacgcgcgaggcgcaggcggtggcccaagagtggaagtgcgctttcatggagacctcggccaagatgaactacaacgtcaaggagctcttccaggagctgctgacgctggagacgcgccggaacatgagcctcaacatcgacggcaagcgctccgggaagcagaagaggacagaccgcgtcaagggcaaatgcaccctcatgtga

SEQ ID NO: 5 - protein, 198 aa, Homo sapiens
MPEQSNDYRVVVFGAGGVGKSSLVLRFVKGTFRDTYIPTIEDTYRQVISCDKSVCTLQITDTTGSHQFPAMQRLSISKGHAFILVFSVTSKQSLEELGPIYKLIVQIKGSVEDIPVMLVGNKCDETQREVDTREAQAVAQEWKCAFMETSAKMNYNVKELFQELLTLETRRNMSLNIDGKRSGKQKRTDRVKGKCTLM

SEQ ID NO: 6 - DNA, 21 bp, Artificial Sequence
  Synthetic
gcgtgggcaagaactcgctgg

SEQ ID NO: 7 - DNA, 21 bp, Artificial Sequence
  Synthetic
ccagcgagttcttgcccacgc

SEQ ID NO: 8 - protein, 20 aa, Artificial Sequence
  Synthetic
LNIDGKRSGKQKRTDRVKGK

SEQ ID NO: 9 - protein, 229 aa, Homo sapiens
MGNASFGSKEQKLLKRLRLLPALLILRAFKPHRKIRDYRVVVVGTAGVGKSTLLHKWASGNFRHEYLPTIENTYCQLLGCSHGVLSLHITDSKSGDGNRALQRHVIARGHAFVLVYSVTKKETLEELKAFYELICKIKGNNLHKFPIVLVGNKSDDTHREVALNDGATCAMEWNCAFMEISAKTDVNVQELFHMLLNYKKKPTTGLQEPEKKSQMPNTTEKLLDKCIIM

SEQ ID NO: 10 - protein, 206 aa, Homo sapiens
MAANKPKGQNSLALHKVIMVGSGGVGKSALTLQFMYDEFVEDYEPTKADSYRKKVVLDGEEVQIDILDTAGQEDYAAIRDNYFRSGEGFLCVFSITEMESFAATADFREQILRVKEDENVPFLLVGNKSDLEDKRQVSVEEAKNRAEQWNVNYVETSAKTRANVDKVFFDLMREIRARKMEDSKEKNGKKKRKSLAKRIRERCCIL

SEQ ID NO: 11 - protein, 184 aa, Homo sapiens
MREYKLVVLGSGGVGKSALTVQFVQGIFVEKYDPTIEDSYRKQVEVDCQQCMLEILDTAGTEQFTAMRDLYMKNGQGFALVYSITAQSTFNDLQDLREQILRVKDTEDVPMILVGNKCDLEDERVVGKEQGQNLARQWCNCAFLESSAKSKINVNEIFYDLVRQINRKTPVEKKKPKKKSCLLL

SEQ ID NO: 12 - protein, 183 aa, Homo sapiens
MREYKVVVLGSGGVGKSALTVQFVTGTFIEKYDPTIEDFYRKEIEVDSSPSVLEILDTAGTEQFASMRDLYIKNGQGFILVYSLVNQQSFQDIKPMRDQIIRVKRYEKVPVILVGNKVDLESEREVSSSEGRALAEEWGCPFMETSAKSKTMVDELFAEIVRQMNYAAQPDKDDPCCSACNIQ

SEQ ID NO: 13 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 14 - protein, 218 aa, Homo sapiens
MSSGAASGTGRGRPRGGGPGPGDPPPSETHKLVVVGGGGVGKSALTIQFIQSYFVSDYDPTIEDSYTKICSVDGIPARLDILDTAGQEEFGAMREQYMRAGHGFLLVFAINDRQSFNEVGKLFTQILRVKDRDDFPVVLVGNKADLESQRQVPRSEASAFGASHHVAYFEASAKLRLNVDEAFEQLVRAVRKYQEQELPPSPPSAPRKKGGGCPCVLL

SEQ ID NO: 15 - protein, 184 aa, Homo sapiens
MPQSKSRKIAILGYRSVGKSSLTIQFVEGQFVDSYDPTIENTFTKLITVNGQEYHLQLVDTAGQDEYSIFPQTYSIDINGYILVYSVTSIKSFEVIKVIHGKLLDMVGKVQIPIMLVGNKKDLHMERVISYEEGKALAESWNAAFLESSAKENQTAVDVFRRIILEAEKMDGAASQGKSSCSVM

(19) United States
(12) Patent Application Publication
(10) Pub. No.: US 2003/0059771 A1
(43) Pub. Date: Mar. 27, 2003

(54) Compositions and methods related to the Rig tumor suppressor gene and protein

(75) Inventors: Geoff J. Clark, Gaithersburg, MD (US); Chad A. Ellis, Rockville, MD (US); Michelle Vos, Boyds, MD (US)

Correspondence Address: MEDLEN & CARROLL, LLP, 101 HOWARD STREET, SUITE 350, SAN FRANCISCO, CA, 94105, US

(73) Assignee: THE UNITED STATES OF AMERICA, Department of Health and Human Services

(21) Appl. No.: 09/873,546

(22) Filed: Jun. 4, 2001

(51) Int. Cl. C12Q 1/68; C07H 21/04; C12N 9/10; C12P 21/02; C12N 5/06

(52) U.S. Cl. 435/6; 435/193; 435/320.1; 435/325; 530/388.26; 536/23.2

(57) ABSTRACT

The present invention relates to Ras-family proteins and tumor suppressor genes. Specifically, the present invention provides the Ras-related tumor suppressor gene and protein Rig, as well as means for the regulation of cell growth.

This invention was made during the course of work supported by the United States Government, under the National Cancer Institute. As such, the United States Government may have certain rights to this invention.

### FIELD OF THE INVENTION

The present invention relates to Ras-family proteins and tumor suppressor genes. Specifically, the present invention relates to the Ras-related tumor suppressor gene and protein Rig, and the regulation of cell growth. The invention provides compositions and methods for the analysis of Rig gene structure, transcription, expression and function, as well as the treatment of cancer.

### BACKGROUND OF THE INVENTION

Ras superfamily proteins, also called “small GTP-binding proteins” are utilized by all eukaryotes to transduce extracellular signals which regulate basic cellular functions. These diverse pathways mediate pleiotropic effects ranging from growth and transformation to membrane and cytoskeletal rearrangements to cell survival and apoptosis in multiple tissues and during development. The integral role played by Ras proteins in regulating signal transduction, gene expression, cell growth and transformation are widespread.

The Ras (also known as “p21”) oncoproteins are prototypical members of the large, structurally related Ras superfamily of monomeric GTP-binding proteins. In the best studied Ras-mediated signal transduction pathways, Ras is activated by receptor tyrosine kinases (RTK), which are located at the cell membrane. The Ras proteins serve as molecular switches, cycling between active and inactive states, integrating extracellular signals into cellular responses. There exists a wide variety of RTK proteins, which receive and transmit extracellular signals, that in turn activate the Ras proteins. Activated Ras proteins then, in turn, activate other signaling proteins resulting in highly regulated and specific signaling cascades (Katz and McCormick, Curr. Opin. Genet. Dev., 7:75-79 [1997]; Campbell et al., Oncogene 17:1395-1413 [1998]; and Malumbres and Pellicer, Front Biosci 3:d887-d912 [1998]). The downstream components of many of these signalling cascades remain unidentified.

Activated Ras proteins mediate a broad range of biological effects, many of which are associated with enhanced growth and transformation. These effects include reduced growth factor dependence (Andrejauskas and Moroni, EMBO J., 8:2575-2581 [1989]), the induction of DNA synthesis (Mulcahy et al., Nature 313:241-243 [1985]), loss of contact inhibition (Huber and Cordingley, Oncogene 3:245-256 [1988]), inhibition of terminal differentiation (Yuspa et al., Nature 314:459-462 [1985]), resistance to apoptosis (Kauffmann-Zeh et al., Nature 385:544-548 [1997]), enhanced motility (Trahey et al., Mol. Cell Biol., 7:541-544 [1987]), metastasis/invasion (Ochieng et al., Invasion Metastasis 11:38-47 [1991]; and Takiguchi et al., Clin. Exp. Metastasis 10:351-360 [1992]) and tumorigenic transformation (Barbacid, Annu. Rev. Biochem., 56:779-827 [1987]; and Lowy and Willumsen, Annu. Rev. Biochem., 62:851-891 [1993]).

The signaling activity of the Ras protein is modulated by its bound guanine nucleotide. Ras proteins which bind the trinucleotide GTP are in an active conformation, while Ras proteins which bind the dinucleotide GDP are inactive (McCormick, Nature 363:15 [1993]; and Marshall, Curr. Opin. Genet. Dev., 4(1):82-92 [1994]). Following the binding of GTP, intrinsic Ras GTPase activity hydrolyses the terminal phosphate of GTP to yield GDP, which is subsequently exchanged for GTP. The GTPase and nucleotide exchange activities intrinsic to Ras are augmented by several regulatory proteins. The ancillary proteins, Ras-GTPase activating protein (GAP) and guanine nucleotide exchange factor (GNEF), are important modulators of Ras-signaling.

All Ras proteins contain conserved structural domains critical for normal biological function, including sequence motifs necessary for guanine nucleotide binding and GTPase activity. Ras proteins also have a conserved effector domain enabling them to interact with a multitude of downstream signaling molecules. Ras proteins ultimately exert their influence by activating a myriad of effector molecules such as Raf-1 and phosphatidylinositol-3-kinase (PI3K), leading to MAPK and PKB/Akt activation, respectively.

Mammalian cells are known to have at least three Ras proteins, namely, H-Ras, K-Ras and N-Ras. These Ras proteins, although sharing a highly conserved structure, have been shown to serve different functions within the cell. In addition, there are families of more distantly related small GTP-binding proteins, including Rac, Rho, CDC42, TC21, Rit, Ral, and Rap (Campbell et al., Oncogene 17:1395-1413 [1998]; and Malumbres and Pellicer, Front Biosci 3:d887-d912 [1998]).

For proper membrane association and biological function, Ras proteins (and most Ras-related proteins) must undergo a series of post-translational modifications resulting in carboxy-terminus isoprenylation by a farnesyl-lipid moiety (Bar-Sagi, Mol Cell Biol 21:1441-1443 [2001]. Nearly all Ras-related proteins possess a terminal CAAX (SEQ ID NO: 1) motif (cysteine-aliphatic-aliphatic-X) that serves as a signal for sequential isoprenylation, proteolytic cleavage of the -AAX group and carboxymethylation of the now terminal cysteine. The X amino acid residue determines the type of covalent isoprenylation.

This covalent modification is catalyzed by a family of enzymes called farnesyl transferases. Drugs that target and inactivate farnesyl transferase enzymes can inhibit the farnesylation of Ras proteins and disrupt Ras activity. Drugs that specifically inhibit the farnesyl transferase enzymes are called Farnesyl Transferase Inhibitor (FTI) compounds.

Ras Signaling in Cancer

Activated Ras proteins play a key role in the development of human cancers. Mutations in Ras are observed in approximately one third of all tumors, underscoring their importance (Bos, Cancer Res 49:4682-4689 [1989]; and Clark and Der, in GTPases in Biology [eds. Dickey and Birmbauer], Springer-Verlag London Ltd., pp. 259-287 [1993]). Indeed, the Ras mutation frequency approaches 100% in some types of tumors (e.g., pancreatic adenocarcinoma). These mutated Ras proteins demonstrate decreased inherent GTPase activity, and are resistant to the action of GTPase-activating proteins (GAPs). Thus, these mutations are activating mutations resulting in the Ras protein being locked in an active conformation, leading ultimately to inappropriate cell proliferation signals. Furthermore, activated forms of the Ras protein are able to induce tumors in animal models, thereby providing direct evidence for Ras involvement in malignant cell transformation and tumorigenesis. Moreover, deletion of the activated Ras gene from tumor cell lines impairs their tumorigenicity (Paterson et al., Cell 51:803-812 [1987]; and Shirasawa et al., Science 260:85-88 [1993])

In addition to the role Ras proteins play in oncogenesis, Ras proteins can also function as tumor suppressors. For example, some Rap and Rheb proteins largely antagonize Ras signaling and transformation. Furthermore, a Ras superfamily member, Noey2/Ahri1, functions as a tumor suppressor in breast and ovarian tissue (Yu et al., Proc. Natl. Acad. Sci. USA 96:214-219 [1999]). Thus, despite amino acid sequence homology, Ras superfamily proteins are functionally diverse, exhibiting both oncogenic and tumor suppressor properties.

There is a need in the art to identify genes and proteins involved in the regulation of cell proliferation and a need for improved understanding of Ras-family proteins in order to elucidate the molecular basis of cancer. There is also a need in the art for compositions and methods which have the ability to control unregulated or harmful cell proliferation. Such compositions and methods have therapeutic value. For example, such compositions and methods find use in the eradication and/or suppression of tumors.

### SUMMARY OF THE INVENTION

The present invention relates to a Ras-related Inhibitor of Growth (Rig) gene and protein. Rig is a Ras family member with tumor suppressor properties. In particular, the present invention provides compositions and methods for the analysis of Rig gene structure, expression and function. It is contemplated that Rig will find use in the diagnosis and treatment of cancer, and most preferably, for the treatment of tumors that show deletion or mutation of the endogenous Rig gene and/or reduced expression of the Rig transcript or protein.

In one embodiment, the present invention provides a recombinant expression vector comprising the nucleotide sequence encoding the polypeptide set forth in SEQ ID NO:5 (i.e., the Rig protein). In a preferred embodiment, this nucleotide sequence comprises the nucleotide sequence set forth in SEQ ID NO:4 (i.e. Rig cDNA). In related embodiments, the recombinant expression vector comprises a replication defective virus. In other embodiments, a host cell transfected with said recombinant expression vector is provided, wherein the host cell is either prokaryotic (i.e., bacterial) or eukaryotic (e.g., mammalian).

The present invention also provides antibodies directed against Rig, or any portion of the Rig polypeptide. In some embodiments, the antibody is monoclonal, while in other embodiments, the antibody is polyclonal. In a related embodiment, the invention provides compositions comprising an anti-Rig antibody.

Moreover, the present invention provides methods for detecting Rig nucleotide sequences in a sample by using a probe comprising nucleic acids having complementarity to a portion of the nucleotide sequence set forth in SEQ ID NO: 4. These methods involve hybridization under suitable conditions and detection of the newly-formed hybridization complex. In a preferred embodiment these methods comprise Northern blotting. In some embodiments of this invention, the sample is total RNA, while in other embodiments the sample is polyA-enriched RNA or genomic DNA. In related embodiments, the sample comprises tumor tissue which may be collected from a human subject.

In addition, the present invention provides methods for amplifying Rig nucleic acid sequences. In a most preferred embodiment, this method is polymerase chain reaction (PCR) method comprising: (a) providing a sample, a DNA polymerase, two primers having complementarity to the nucleotide sequence of SEQ ID NO: 4, and amplification reagents; (b) combining the sample, DNA polymerase, primers and amplification reagents; (c) annealing the primers to the template in the sample; (d) extending the primers with reiterated DNA synthesis under conditions such that the template is amplified to produce an amplified PCR product; and (e) detecting the amplified PCR product. In some embodiments the Rig nucleic acid sequences are DNA, while in others the nucleic acid sequences are RNA. In a preferred embodiment, the sample is derived from a human subject, while in a particularly preferred embodiment the sample is derived from tumor tissue. In one preferred embodiment, the DNA polymerase has DNA-dependent DNA polymerase activity, while in an alternate preferred embodiment, the polymerase has both DNA-dependent DNA polymerase activity and reverse transcriptase RNA-dependent DNA polymerase activity. In some embodiments, the primers are oligonucleotides comprising the sequences set forth in SEQ ID NO:2 and SEQ ID NO:3.

The present invention also provides methods for detecting a Rig polypeptide in a sample, comprising: (a) providing a sample and an antibody directed against the sequence set forth in SEQ ID NO:5; (b) contacting the sample with the antibody under conditions such that the antibody specifically binds to the polypeptide in the sample to form an antigen-antibody complex; and (c) detecting the antigen-antibody complex. In one embodiment, the sample is from a human subject. In another embodiment, the sample is tumor tissue. In some embodiments, the method comprises Western immunoblotting, while in other embodiments, the method comprises an enzyme-linked immunosorbent assay (ELISA). In some embodiments, the ELISA is selected from the group consisting of direct ELISA, indirect ELISA, direct sandwich ELISA, indirect sandwich ELISA, and competitive ELISA.

The present invention also provides methods for inhibiting cell growth comprising: (a) providing a cell, a recombinant expression vector comprising a nucleotide sequence encoding the polypeptide set forth in SEQ ID NO:5, a means for delivery of the vector into the cell; (b) delivering the vector into the cell using the delivery means; and (c) expressing the polypeptide within the cell. In a preferred embodiment, the cell is a human cell, while in a particularly preferred embodiment, the human cell is within a human subject (i.e. in vivo). In some embodiments, the cell is a tumor cell which may be of neural origin. In embodiments in which the cell is of neural origin, the cell is selected from the group consisting of astrocytoma, glioblastoma, Ewing sarcoma, primitive neuroectoderm, rhabdomyosarcoma, undifferentiated carcinoma and neuroblastoma cells. In some preferred embodiments, the means of delivery of the vector is selected from the group comprising direct nucleic acid administration, lipofection, recombinant viral delivery and any combination thereof. In a related embodiment, the recombinant virus comprises operably linked nucleotide sequences comprising a suitable promoter sequence and viral sequences, wherein the viral sequences are selected from the group consisting of adenovirus, adeno-associated virus, retrovirus, herpes virus, vaccinia virus and Moloney virus sequences. In some embodiments, the means of delivery is local or systemic. In embodiments in which the delivery is local, it may be accomplished by surgical delivery, implantation or injection.

In one embodiment, the present invention provides a method for treating a subject comprising: (a) providing a subject, a recombinant expression vector comprising the nucleotide sequence encoding the polypeptide set forth in SEQ ID NO:5 (i.e., the Rig protein), a target within the subject, and a means of delivery of the vector to the target within the subject; and (b) delivering the vector to the target within the subject. In one preferred embodiment, the subject is a human and in another preferred embodiment the target is a solid tumor. In some embodiments, the tumor is of neural origin and in a related embodiment the neural tumor is an astrocytoma. Moreover, in some embodiments, the means of vector delivery is selected from the group consisting of direct nucleic acid administration, lipofection, recombinant viral infection, and any combination thereof. In embodiments including a recombinant virus, the virus comprises operably linked recombinant nucleotide sequences such as a promoter and viral sequences selected from the group consisting of adenovirus, adeno-associated virus, retrovirus, herpes virus, vaccinia virus, and Moloney virus sequences. Also, in some embodiments, the means of delivery is selected from the group consisting of local and systemic delivery, wherein the local delivery means is selected from the group comprising surgical delivery, implantation and injection.

### DESCRIPTION OF THE FIGURES

FIG. 1 shows the sequence of the Rig gene and the predicted sequence of the Rig protein. Panel A shows the nucleotide sequence of the human Rig open reading frame (SEQ ID NO:4). Panel B shows the predicted amino acid sequence of the human Rig open reading frame (GenBank Accession No. AAD13119; and SEQ ID NO:5).

FIG. 2 shows an alignment of the Rig amino acid sequence with the amino acid sequences of other human Ras-family members. In this Figure, the Rig protein guanine nucleotide binding domain is indicated in bold capital letters, the effector binding domain is indicated by underlined capital letters, and the CAAX (SEQ ID NO: 1) motif is simply capitalized.

FIG. 3 shows a dendogram analysis of sequence divergence of various Ras family proteins, including Rig.

FIG. 4 shows a multi-tissue mRNA analysis. Panel A shows a human poly(A)⁺ RNA Northern blot (Clontech) probed with a Rig cDNA probe. Panel B shows a human poly(A)⁺ RNA Northern slot blot (Clontech) probed with a Rig cDNA probe. Panel C shows the key to the RNA samples in the slot blot.

FIG. 5 shows Northern blot results for total RNA from tumor-derived neural cell lines examined with a Rig cDNA probe (top panel). The bottom panel of the Figure shows 28S ribosomal RNA staining of the same blot, which was used for loading-normalization.

FIG. 6 shows the results of a clonogenic focus formation assay and an immunoblot control. Panel A shows the results of the clonogenic focus formation assay using NIH-3T3 cells stably transfected with vectors expressing wild-type and mutant Rig proteins. Panel B shows an immunoblot analysis of the transfected NIH-3T3 cell line lysates tested using an anti-HA primary antibody.

FIG. 7 shows the results of an Elk-1-dependent luciferase transient transfection assay, as a function of increasing Rig expression. The Elk-i transcriptional activity was determined using a GAL4 DNA binding luciferase reporter system.

FIG. 8 shows the results of a Ras-transformation focus formation assay using NIH-3T3 cells. The effects of Rig and Rap(Q63E) expression on Ras transformation are indicated.

FIG. 9 shows the results of an in vivo co-immunoprecipitation assay, where various proteins were tested for their ability to interact with the Raf-1 protein in HEK-293T cells. An anti-Raf antibody was used in the immunoprecipitation, and anti-FLAG and anti-HA antibodies used, alternatively, in the immunodetection step. Rig, H-Ras and K-Ras were tested in the assay.

FIG. 10 shows the results of cellular growth rate assays and immunoblot analyses. Panel A shows the results of a cellular growth rate assay, where the growth rate (as determined by enumerating cells at multiple time points) of U251 and A673 neural tumor cell lines was observed to be a function of Rig expression levels. This experiment employed a pLRT-based inducible expression system, where addition of doxycycline (dox) results in Rig gene expression. Untreated control cultures were also assayed. Panel B shows a Western blot of lysates from experimental cell lines following drug induction. This blot was probed with anti-Rig antiserum.

FIG. 11 shows the results of a Western blot analysis of normal and tumor cell lysates. The lysates were prepared from human neural tissue (normal striatum), oligodendroglioma (05 and 011), and end-stage astrocytoma (U15, U42 and U49) tumor specimens. The blot in the top panel was probed with an anti-Rig polyclonal antiserum. The same blot was then stripped and probed with a β-tubulin-reactive antibody for loading control purposes, as shown in the lower panel.

FIG. 12 shows the results of a Western blot analysis of normal and tumor cell lysates. The blotted material was prepared from a series of human astrocytoma tumor samples and the blot was probed with an anti-Rig polyclonal antiserum. The tumor samples were derived from two clinical groups, low-grade tumors (13 samples) and high-grade tumors (6 samples). Normal human striatum tissue was also analyzed.

FIG. 13 shows the results of a Western blot analysis of human and murine neural tissue lysates. The blot was probed with an anti-Rig polyclonal antiserum. The positions to which molecular size markers traveled are shown in the center lane.

FIG. 14 shows the results of an in vitro prenylation assay. In panel A, the prenylation of GST-purified Ras, Rap and Rig proteins was examined using radiolabelled forms of farnesyl pyrophosphate, geranylgeranyl pyrophosphate or mevalonate as substrates. In panel B, the prenylation of GST-Ras, GST-Rap and GST-Rig by mevalonate in the presence of various concentrations of farnesyl transferase inhibitor was examined.

FIG. 15 shows fluorescence microscopic images of the E293 cell line engineered to conditionally express GFP alone or a GFP-Rig fusion protein through the use of a pIND(SP1)/pVgRXR two part, ecdysone-inducible mammalian conditional expression system.

### GENERAL DESCRIPTION OF THE INVENTION

The present invention relates to a novel Ras-superfamily gene having tumor suppressor activity. Specifically, the present invention provides the human Rig gene and the protein encoded by this gene. In addition, the present invention also provides recombinant vectors comprising the gene, host cells comprising the vectors and antibodies specific for the Rig protein, as well as other compositions.

The compositions of the present invention find use in the detection of the Rig gene, transcript and/or protein for research and cancer diagnosis. Furthermore, the compositions of the present invention find use in methods for inhibiting cell growth, and in the treatment of cancer, where the Rig gene is delivered to the cancer cells of a subject.

An understanding of the mechanism(s) of Rig activity is not required in order to make or use the present invention. Furthermore, is it not intended that the present invention be limited to any particular mechanism(s).

Definitions

To facilitate understanding of the invention, a number of terms are defined and discussed below.

The terms “nucleic acid,” “nucleic acid sequence,” “nucleotide sequence,” “oligonucleotide,” “polynucleotide” or “nucleic acid molecule” as used herein refer to an oligonucleotide or polynucleotide, and fragments or portions thereof, and to DNA or RNA of genomic or synthetic origin which can be single- or double-stranded, and represent the sense or antisense strand. Similarly, “amino acid sequence” as used herein refers to the primary sequence of amino acids in a peptide, polypeptide or protein.

The term “nucleotide” as used herein refers to any nucleotide that comprises any of the known base analogs of DNA and RNA including, but not limited to, 4-acetylcytosine, 8-hydroxy-N6-methyladenosine, aziridinylcytosine, pseudoisocytosine, 5-(carboxyhydroxyhnethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “oligonucleotide,” refers to a short length of single-stranded polynucleotide chain. Oligonucleotides are typically less than 100 nucleotides long (e.g., between 15 and 50), however, as used herein, the term is also intended to encompass longer polynucleotide chains. Oligonucleotides are often referred to by their length. For example a 24 residue oligonucleotide is referred to as a “24-mer.” Oligonucleotides can form secondary and tertiary structures by self-hybridizing or by hybridizing to other polynucleotides. Such structures include, but are not limited to, duplexes, hairpins, cruciforms, bends, and triplexes.

As used herein, “recombinant nucleic acid,” “recombinant gene” “recombinant DNA molecule” or similar terms indicate that the nucleotide sequence or arrangement of its parts is not in a native configuration, and has been manipulated by molecular biological techniques. The term implies that the DNA molecule is comprised of segments of DNA that have been artificially joined together. Protocols and reagents to manipulate nucleic acids are common and routine in the art (See e.g., Maniatis et al. (eds.), Molecular Cloning: A Laboratory Manual, Cold Spring Harbor Laboratory Press, NY, [1982]; Sambrook et al. (eds.), Molecular Cloning: A Laboratory Manual, Second Edition, Volumes 1-3, Cold Spring Harbor Laboratory Press, NY, [1989]; and Ausubel et al. (eds.), Current Protocols in Molecular Biology, Vol. 1-4, John Wiley & Sons, Inc., New York [1994]).

As used herein, the terms “restriction endonucleases” and “restriction enzymes” refer to bacterial enzymes, each of which cut double-stranded DNA at or near a specific nucleotide sequence.

As used herein, the term “probe” refers to an oligonucleotide (i.e., a sequence of nucleotides), which is often produced from nucleic acid isolated from cells (typically a recombinant nucleic acid), produced synthetically or in vitro, which is capable of hybridizing to a nucleic acid of interest. Probes are useful in the detection, identification and isolation of particular gene sequences. It is contemplated that any probe used in the present invention is capable of being labelled with any “reporter molecule,” so that the probe is detectable. Detection systems include, but are not limited to, the detection of enzymatic activity, fluorescence, radioactivity, and luminescence. It is not intended that the present invention be limited to any particular probe, label or detection system.

As used herein, the terms “complementary” or “complementarity” are used in reference to antiparallel polynucleotides (i.e., a sequence of nucleotides) related by the base-pairing rules. For example, the sequence 5′-AGTTC-3′ is complementary to the sequence 3′-TCAAG-5′. Complementarity can be “partial,” in which only some of the nucleic acids' bases are matched according to the base pairing rules. Or, there can be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “homology,” as it applies to nucleotide sequences, refers to a degree of complementarity. It is intended that the term encompass partial homology as well as complete homology (i.e., 100% identity). A partially complementary sequence is one that at least partially inhibits a completely complementary sequence from hybridizing to a target nucleic acid, and is referred to using the functional term “substantially homologous.” The inhibition of hybridization of the completely complementary sequence to the target sequence can be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding can be tested by the use of a second target which lacks even a partial degree of complementarity (e.g., less than about 30% identity); in the absence of non-specific binding, the probe will not hybridize to the second non-complementary target.

The term “hybridization” as used herein includes “any process by which a strand of nucleic acid joins with a complementary strand through base pairing” (Coombs, Dictionary of Biotechnology, Stockton Press, New York NY [1994]. Hybridization can be demonstrated using a variety of hybridization assays (Southern blot, Northern Blot, slot blot, phage plaque hybridization, and other techniques). These protocols are common in the art (See e.g., Sambrook et al. (eds.), Molecular Cloning: A Laboratory Manual, Second Edition, Volumes 1-3, Cold Spring Harbor Laboratory Press, NY, [1989]; Ausubel et al. (eds.), Current Protocols in Molecular Biology, Vol. 1-4, John Wiley & Sons, Inc., New York [1994]).

Hybridization may occur between two antiparallel nucleic acids which may or may not have 100% complementarity. Two nucleic acids which contain 100% antiparallel complementarity will show strong hybridization. Two antiparallel nucleic acids which contain no antiparallel complementarity (generally considered to be less than 30%) will not hybridize. Two nucleic acids which contain between 31-99% complementarity will show an intermediate level of hybridization. A single molecule that contains pairing of complementary nucleic acids within its structure is said to be “self-hybridized.”

As used herein, the term “stringency” is used in reference to the conditions of temperature, ionic strength, and the presence of other compounds such as organic solvents, under which nucleic acids hybridize. “Low or weak stringency” conditions are reaction conditions which favor the complementary base pairing and annealing of two nucleic acids. “High stringency” conditions are those conditions which are less optimal for complementary base pairing and annealing. The art knows well that numerous variables affect the strength of hybridization and that numerous equivalent conditions can be employed to comprise either low or high stringency conditions. Specifically, factors such as the length and composition of the probe (DNA, RNA, nucleotide sequence) and composition of the target (DNA, RNA, nucleotide sequence, present in solution or immobilized, the degree of complementary between the nucleic acids, etc.) influence the strength of the nucleic acid hybridization complex. In addition, the concentration of salts and other components (e.g., the presence or absence of formamide, dextran sulfate, polyethylene glycol), temperature and the number and volume of the wash steps also must be considered in selecting suitable hybridization conditions. Conditions which constitute high or low stringency are common to one familiar with the art, and are described in numerous sources (e.g., Anderson and Young, Quantitative Filter Hybridization, in Nucleic Acid Hybridization [1985] and Ausubel et al. (eds.), Current Protocols in Molecular Biology, Vol. 1-4, John Wiley & Sons, Inc., New York [1994]).

During hybridization of two nucleic acids under high stringency conditions, complementary base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less. As used herein, two nucleic acids which are able to hybridize under high stringency conditions are considered “substantially homologous.”

“Stringency” typically occurs in a range from about Tₘ−5° C. (i.e., 5° C. below the Tₘ of the probe) to about 20° C. to 25° C. below Tₘ. As will be understood by those of skill in the art, a stringent hybridization can be used to identify or detect identical polynucleotide sequences or to identify or detect similar or related polynucleotide sequences.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value can be calculated by the equation: Tₘ=81.5+0.41 (% G+C), when a nucleic acid is in aqueous solution at 1 M NaCl (See e.g., Anderson and Young, supra). Other references known to those in the art include more sophisticated computations which take structural as well as sequence characteristics into account for the calculation of Tₘ.

Whether sequences are “substantially homologous” can be verified using hybridization competition assays. For example, a “substantially homologous” nucleotide sequence is one that at least partially inhibits a completely complementary probe sequence from hybridizing to a target nucleic acid under conditions of low stringency. This is not to say that conditions of low stringency are such that non-specific binding is permitted; low stringency conditions require that the binding of two sequences to one another be a specific (i.e., selective) interaction. The absence of non-specific binding can be verified by the use of a second target that lacks even a partial degree of complementarity (e.g., less than about 30% identity); in the absence of non-specific binding the probe will not hybridize to the second non-complementary target. When used in reference to a double-stranded nucleic acid sequence such as a cDNA or genomic clone, the term “substantially homologous” refers to any probe that is capable of hybridizing to either or both strands of the double-stranded nucleic acid sequence under conditions of high stringency.

A gene may produce multiple RNA species that are generated by differential splicing of the primary RNA transcript. cDNAs that are splice variants of the same gene contain regions of nucleotide sequence identity (100% homology), representing the presence of the same exon or portion of the same exon on both cDNAs, and regions of non-identity. The two cDNAs contain regions of nucleotide sequence that will hybridize to a probe derived from the entire gene or portions of the gene containing sequences found on both cDNAs. As used herein, the two splice variants are therefore substantially homologous to such a probe and to each other.

As used herein the term “hybridization complex” refers to a complex formed between two nucleic acid sequences by virtue of the formation of hydrogen bonds between complementary G and C bases and between complementary A and T bases; these hydrogen bonds can be further stabilized by base stacking interactions. The two complementary nucleic acid sequences hydrogen bond in an antiparallel configuration. A hybridization complex can be formed in solution or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH [fluorescent in situ hybridization]).

As used herein, the term “antisense” is used in reference to any nucleic acid which is antiparallel to and complementary to another nucleic acid. The present invention encompasses antisense DNA and RNA produced by any method. For example, in some embodiments, a cDNA or a portion of a cDNA is subcloned into an expression vector containing a promoter which permits transcription either in vitro or in vivo. The cDNA or a portion of the cDNA is subcloned in such a way that it is in the reverse orientation relative to the direction of transcription of the cDNA in its native chromosome. Transcription of this antisense cDNA produces an RNA transcript that is complementary and antiparallel to the native mRNA. In alternative embodiments, the antisense nucleic acid is a synthetically-produced oligonucleotide. The mechanism by which an antisense nucleic acid produces effects in a biological system is unclear. In some embodiments, antisense techniques are used to produce an “artificial knockout” mutant in an animal or animal cell line. The term “antisense strand” is used in reference to the nucleic acid strand that is complementary to the “sense” strand. The designation (−) (i.e., “negative”) is sometimes used in reference to the antisense strand, with the designation (+) (i.e., “positive”) sometimes used in reference to the sense strand.

“Amplification” is defined as the production of additional copies of a nucleic acid sequence and is generally carried out using polymerase chain reaction (PCR) or other technologies well known in the art (e.g., Dieffenbach and Dveksler, PCR Primer, a Laboratory Manual, Cold Spring Harbor Press, Plainview N.Y. [1995]). As used herein, the term “polymerase chain reaction” (“PCR”) refers to the methods of U.S. Pat. Nos. 4,683,195, 4,683,202 and 4,965,188 (hereby incorporated by reference) for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension (DNA synthesis) are typically reiterated numerous times (i.e., denaturation, annealing and extension constitute one “cycle”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

As used herein, the term “polymerase” refers to any polymerase suitable for use in the amplification of nucleic acids of interest. It is intended that the term encompass such DNA polymerases as Taq DNA polymerase obtained from Thermus aquaticus, although other polymerases, both thermostable and thermolabile, are also encompassed by this definition.

As used herein, the term “primer” refers to an oligonucleotide, typically but not necessarily produced synthetically, that is capable of acting as a point of initiation of nucleic acid synthesis when placed under conditions in which synthesis of a primer extension product that is complementary to a nucleic acid strand is induced, (i.e., in the presence of nucleotides, an inducing agent such as DNA polymerase, and at a suitable temperature and pH). The primer is preferably single stranded for maximum efficiency in amplification, but in alternative embodiments, it is double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, primer source and method.

As used herein, the term “nested primers” refers to primers that anneal to the target sequence in an area that is inside the annealing boundaries used to initiate PCR. (See, K. B. Mullis, et al., Cold Spring Harbor Symposia, Vol. LI, pp. 263-273 [1986]). Because the nested primers anneal to the target inside the annealing boundaries of the starting primers, the predominant PCR-amplified product of the starting primers is necessarily a longer sequence, than that defined by the annealing boundaries of the nested primers. The PCR-amplified product of the nested primers is an amplified segment of the target sequence that cannot, therefore, anneal with the starting primers. As used herein, the term “amplification reagents” refers to those reagents (deoxyribonucleoside triphosphates, buffer, etc.), needed for amplification except for primers, nucleic acid template and the amplification enzyme.

Using PCR and an appropriate set of primer molecules, it is possible to amplify a single copy of a specific target sequence in genomic DNA, cDNA, mRNA or any other nucleic acid, to a level detectable by several different methodologies (e.g., ethidium bromide visualization; hybridization with a labelled probe; incorporation of biotinylated primers followed by avidin-enzyme conjugate detection and incorporation of ³²P-labeled deoxynucleotide triphosphates, such as dCTP or dATP, into the amplified segment). In particular, the amplified segments created by the PCR process itself are, themselves, efficient templates for subsequent PCR amplifications. Amplified target sequences are useful to obtain segments of DNA (e.g., genes) for insertion into recombinant vectors.

As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences.

The terms “peptide,” “polypeptide” and “protein” all refer to a primary sequence of amino acids that are joined by covalent “peptide linkages.” In general, a peptide consists of a few amino acids, typically from 2-25 amino acids, and is shorter than a protein. “Polypeptides” encompass both peptides or proteins. As used herein, a recited “amino acid sequence” refers to an amino acid sequence of a naturally occurring protein molecule, a protein produced by recombinant molecular genetic techniques, or a synthetic or naturally occurring peptide, and may refer to a portion of a larger “peptide,” “polypeptide” or “protein,” and is not meant to limit the amino acid sequence to the complete, native amino acid sequence associated with the recited protein molecule.

A “recombinant protein” or “recombinant polypeptide” refers to a protein molecule that is expressed from a recombinant DNA molecule. Use of these terms indicates that the primary amino acid sequence, arrangement of its domains or nucleic acid elements which control its expression are not native, and have been manipulated by molecular biology techniques. As indicated above, techniques to manipulate recombinant proteins are also common and routine in the art.

The terms “exogenous” and “heterologous” are sometimes used interchangeably with “recombinant.” An “exogenous nucleic acid,” “exogenous gene” and “exogenous protein” indicate a nucleic acid, gene or protein, respectively, that has come from a source other than its native source, and has been artificially supplied to the biological system. In contrast, the terms “endogenous protein,” “native protein,” “endogenous gene,” and “native gene” refer to a protein or gene that is native to the biological system, species or chromosome under study. A “native” or “endogenous” gene is a gene that does not contain nucleic acid elements encoded by sources other than the chromosome on which it is normally found in nature. An endogenous gene or transcript is encoded by its natural chromosomal locus, and is not artificially supplied to the cell.

As used herein the term “portion” when in reference to a protein (as in “a portion of a given protein”) refers to fragments of that protein. In some embodiments, the fragments range in size from four amino acid residues to the entire amino acid sequence minus one amino acid. In other embodiments, the “portion” is further limited to only fragments of the full length protein that retain biological activity. For example, a portion of the Rig protein is a fragment of the Rig protein that retains the ability to inhibit Ras activity. Whether or not a fragment of the Rig protein retains the ability to inhibit Ras activity can be determined by use of any suitable functional assay, as provided by the present invention, including by not limited to growth inhibition assays, focus formation assays or Elk-1 luciferase assays.

The following definitions are the commonly accepted definitions of the terms “identity,” “similarity” and “homology.” Percent identity, as it applies to polypeptides, is a measure of strict amino acid conservation. Percent similarity is a measure of amino acid conservation which incorporates both strictly conserved amino acids, as well as “conservative” amino acid substitutions, where one amino acid is substituted for a different amino acid having similar chemical properties (i.e., a “conservative” substitution). In some embodiments, the term “homology” pertains to either proteins or nucleic acids. Two proteins be described as “homologous” or “non-homologous,” but the degree of amino acid conservation is quantitated by percent identity and percent similarity. Nucleic acid conservation is measured by the strict conservation of the bases adenine, thymine, guanine and cytosine in the primary nucleotide sequence. When describing nucleic acid conservation, conservation of the nucleic acid primary sequence is sometimes expressed as percent homology. In the same nucleic acid, one region may show a high percentage of nucleotide sequence conservation, while a different region may show little or no conservation. It is not possible to infer nucleotide sequence conservation from an amino acid similarity score. Indeed, it is possible for two proteins to show domains that in one region are homologous, while other regions of the same protein the domains are non-homologous.

The term “isolated” when used in relation to a nucleic acid, as in “an isolated nucleic acid,” “an isolated oligonucleotide,” “isolated polynucleotide” or “isolated nucleotide sequence,” refers to a nucleic acid that is identified and separated from at least one contaminant nucleic acid with which it is ordinarily associated in its natural source. Isolated nucleic acid is present in a form or setting that is different from the form or setting of that nucleic acid found in nature. In contrast, non-isolated nucleic acids are found in the state in which they exist in nature. For example, a given DNA sequence (e.g., a gene) is found on the host cell chromosome in proximity to neighboring genes; RNA sequences, such as a specific mRNA sequence encoding a specific protein, are found in the cell in a mixture with numerous other mRNAs that encode a multitude of proteins. However, isolated nucleic acid encoding a given polypeptide includes, by way of example, such nucleic acid in cells ordinarily expressing the given protein where the nucleic acid is in a chromosomal location different from that of natural cells, or is otherwise flanked by a different nucleic acid sequence than that found in nature. This isolated nucleic acid, oligonucleotide, or polynucleotide is either single-stranded or double-stranded. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide is single-stranded). In other embodiments, the oligonucleotide or polynucleotide contains both the sense and anti-sense strands (i.e., the oligonucleotide or polynucleotide is double-stranded).

As used herein, the term “purified” or “to purify” refers to the removal of at least one contaminant from a sample. As used herein, the term “substantially purified” refers to molecules, either nucleic acids or amino acid sequences, that are removed from their natural environment, “isolated” or “separated,” and are largely free from other components with which they are naturally associated. An “isolated nucleic acid” or “isolated polypeptide” is therefore a substantially purified nucleic acid or substantially purified polypeptide. For example, antibodies are purified by removal of contaminating non-immunoglobulin proteins; they are also purified by the removal of non-specific immunoglobulin that does not bind to the target molecule. The removal of non-immunoglobulin proteins and/or the removal of immunoglobulins that do not bind to the target molecule results in an increase in the percent of target-reactive immunoglobulins in the sample (i.e., “enrichment” of an antibody). In another example, recombinant polypeptides are expressed in bacterial host cells and the polypeptides are purified by the removal of host cell proteins; the percent of recombinant polypeptides relative to all polypeptides in the sample is thereby increased.

Nucleic acid molecules (e.g., DNA or RNA) are said to have “5′ ends” and “3′ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide, is referred to as the “5′ end” if its 5′ phosphate is not linked to the 3′ oxygen of a mononucleotide pentose ring and as the “3′ end”, if its 3′ oxygen is not linked to a 5′ phosphate of a subsequent mononucleotide pentose ring. As used herein, a nucleic acid sequence, even if internal to a larger oligonucleotide or polynucleotide, also can be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, in some embodiments, enhancer elements exert their effect even when located 3′ of the promoter element or the coding region. Transcription termination and polyadenylation signals are located 3′ or downstream of the coding region.

The term “gene” refers to a nucleic acid (e.g., DNA) sequence comprised of parts, that when appropriately combined in either a native or recombinant manner, provide some product or function. In some embodiments, genes comprise coding sequences necessary for the production of a polypeptide, while in other embodiments, the genes do not comprise coding sequences necessary for the production of a polypeptide. Examples of genes that do not encode polypeptide sequences include ribosomal RNA genes (rRNA) and transfer RNA (tRNA) genes. In preferred embodiments, genes encode a polypeptide or any portion of a polypeptide within the gene's “coding region” or “open reading frame.” In some embodiments, the polypeptide produced by the open reading frame of a gene displays functional activity or properties of the full-length polypeptide (e.g., enzymatic activity, ligand binding, signal transduction, etc.), while in other embodiments, it does not.

In addition to the coding region of the nucleic acid, the term “gene” also encompasses the transcribed nucleotide sequences of the full-length mRNA adjacent to the 5′ and 3′ ends of the coding region. These noncoding regions are variable in size, and typically extend for distances up to or exceeding 1 kb on both the 5′ and 3′ ends of the coding region. The sequences that are located 5′ and 3′ of the coding region and are contained on the mRNA are referred to as 5′ and 3′ untranslated region (5′ UTR and 3′ UTR). Both the 5′ and 3′ UTR may serve regulatory roles, including translation initiation, post-transcriptional cleavage and polyadenylation. The term “gene” encompasses mRNA, cDNA and genomic forms of a gene.

In some embodiments, the genomic form or genomic clone of a gene contains the sequences of the transcribed mRNA, as well as other non-coding sequences which lie outside of the mRNA. The regulatory regions which lie outside the mRNA transcription unit are sometimes called “5′ or 3′ flanking sequences.” A functional genomic form of a gene must contain regulatory elements necessary for the regulation of transcription. The term “promoter/enhancer region” is usually used to describe this DNA region, typically but not necessarily 5′ of the site of transcription initiation, sufficient to confer appropriate transcriptional regulation. Used alone, the term “promoter” is sometimes used synonymously with “promoter/enhancer.” In some embodiments, the promoter is constitutively active, or while in alternative embodiments, the promoter is conditionally active (i.e., where transcription is initiated only under certain physiological conditions or in the presence of certain drugs). In some embodiments, the 3′ flanking region contains additional sequences which regulate transcription, especially the termination of transcription.

“Introns” or “intervening regions” or “intervening sequences” are segments of a gene which are contained in the primary transcript (i.e., hetero-nuclear RNA, or hnRNA), but are spliced out to yield the processed mRNA form. In some embodiments, introns contain transcriptional regulatory elements such as enhancers. The mRNA produced from the genomic copy of a gene is translated in the presence of ribosomes to yield the primary amino acid sequence of the polypeptide.

As used herein, the term “regulatory element” refers to a genetic element which controls some aspect of the expression of nucleic acid sequences. For example, a promoter is a regulatory element that enables the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells, as well as viruses. Analogous control elements (i.e., promoters and enhancers) are also found in prokaryotes. The selection of a particular promoter and enhancer to be operably linked in a recombinant gene depends on what host cell is selected to heterologously express the protein of interest. Some eukaryotic promoters and enhancers have a broad host range while others are functional only in a limited subset of cell types (for review see, Voss et al., Trends Biochem. Sci., 11:287 [1986] and Maniatis et al., Science 236:1237 [1987]). For example, the SV40 early gene enhancer is very active in a wide variety of mammalian cell types (Dijkema et al., EMBO J., 4:761 [1985]). Two other examples of promoter/enhancer elements active in a broad range of mammalian cell types are those from the human elongation factor 1α gene (Uetsuki et al., J. Biol. Chem., 264:5791 [1989]; Kim et al., Gene 91:217 [1990]; Mizushima and Nagata, Nuc. Acids. Res., 18:5322 [1990]), and the long terminal repeats of the Rous sarcoma virus (Gorman et al., Proc. Natl. Acad. Sci. USA 79:6777 [1982]), and human cytomegalovirus (Boshart et al., Cell 41:521[1985]). Some promoter elements serve to direct gene expression in a tissue-specific manner.

As used herein, the term “promoter/enhancer” denotes a segment of DNA which contains sequences capable of providing both promoter and enhancer functions (i.e., the functions provided by a promoter element and an enhancer element). For example, the long terminal repeats of retroviruses contain both promoter and enhancer functions. In some embodiments, the promoter/enhancer is “endogenous,” while in other embodiments, the promoter/enhancer is “exogenous,” or “heterologous.” An “endogenous” promoter/enhancer is one which is naturally linked with a given gene in the genome. An “exogenous” or “heterologous” promoter/enhancer is one placed in juxtaposition to a gene by means of genetic manipulation (i.e., molecular biological techniques such as cloning and recombination) such that transcription of the gene is controlled by the linked promoter/enhancer.

The presence of “splicing signals” on an expression vector often results in higher levels of expression of the recombinant transcript. Splicing signals mediate the removal of introns from the primary RNA transcript and consist of a splice donor and acceptor site (See e.g., Sambrook et al., Molecular Cloning: A Laboratory Manual, Second Edition, Cold Spring Harbor Laboratory Press, New York [1989], pp. 16.7-16.8). A commonly used splice donor and acceptor site is the splice junction from the 16S RNA of SV40.

Efficient expression of recombinant DNA sequences in eukaryotic cells requires the presence of signals directing the efficient termination and polyadenylation of the resulting transcript. Transcription termination signals are generally found downstream of the polyadenylation signal and are generally a few hundred nucleotides in length. The term “poly A site” or “poly A sequence” as used herein denotes a nucleic acid sequence that directs both the termination and polyadenylation of the nascent RNA transcript. Efficient polyadenylation of the recombinant transcript is desirable as transcripts lacking a poly A tail are unstable and are rapidly degraded. In some embodiments, the poly A signal utilized in an expression vector is “heterologous,” while in other embodiments, it is “endogenous.” An endogenous poly A signal is one that is found naturally at the 3′ end of the coding region of a given gene in the genome. A heterologous poly A signal is one that is isolated from one gene and placed 3′ of another gene. A commonly used heterologous poly A signal is the SV40 poly A signal. The SV40 poly A signal is contained on a 237 bp BamHI/BclI restriction fragment and directs both termination and polyadenylation (See e.g., Sambrook et al., Molecular Cloning: A Laboratory Manual, Second Edition, Cold Spring Harbor Laboratory Press, New York [1989], pp.16.6-16.7).

The terms “in operable combination,” “in operable order,” “operably linked” and similar phrases when used in reference to nucleic acid herein are used to refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner that a functional protein is produced.

As used herein, the terms “an oligonucleotide having a nucleotide sequence encoding a gene,” “polynucleotide having a nucleotide sequence encoding a gene,” and similar phrases are meant to indicate a nucleic acid sequence comprising the coding region of a gene (i.e., the nucleic acid sequence which encodes a gene product). In some embodiments, the coding region is present in a cDNA, while in other embodiments, the coding region is present in genomic DNA or RNA form. When present in a DNA form, the oligonucleotide, polynucleotide or nucleic acid is either single-stranded (i.e., the sense strand or the antisense strand) or double-stranded. In some embodiments, suitable control elements such as enhancers/promoters, splice junctions, polyadenylation signals, etc. are placed in close proximity to the coding region of the gene if needed to permit proper initiation of transcription and/or correct processing of the primary RNA transcript. Alternatively, the coding region utilized in the expression vectors of the present invention contains endogenous enhancers/promoters, splice junctions, intervening sequences, polyadenylation signals, etc. or a combination of both endogenous and exogenous control elements.

As used herein, the terms “nucleic acid molecule encoding,” “DNA sequence encoding,” and “DNA encoding” and similar phrases refer to the order or sequence of deoxyribonucleotides along a strand of deoxyribonucleic acid encoding a particular polypeptide. The order of the deoxyribonucleotides determines the order of the amino acids in the polypeptide chain. The DNA sequence thus codes for the amino acid sequence.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of the mRNA. Gene expression regulation often occurs at many stages. “Up-regulation” or “activation” refers to regulation that increases the production of gene expression products (i.e., RNA or protein), while “down-regulation” or “repression” refers to regulation that decreases mRNA or protein production. Molecules (e.g., transcription factors) that are involved in up-regulation or down-regulation are often called “activators” and “repressors,” respectively.

As used herein, the term “vector” is used in reference to nucleic acid molecules that transfer DNA segment(s) from one cell to another. The term “vehicle” is sometimes used interchangeably with “vector.” In some embodiments, a vector “backbone” comprises those parts of the vector which mediate its maintenance and enable its intended use (e.g., the vector backbone contains sequences necessary for replication, genes imparting drug or antibiotic resistance, a multiple cloning site, and possibly operably linked promoter/enhancer elements which enable the expression of a cloned nucleic acid). The cloned nucleic acid (e.g., such as a cDNA coding sequence, or an amplified PCR product) is inserted into the vector backbone using common molecular biology techniques. Vectors are often derived from plasmids, bacteriophages, or plant or animal viruses. A “cloning vector” or “shuttle vector” or “subcloning vector” contain operably linked parts which facilitate subcloning steps (e.g., a multiple cloning site containing multiple restriction endonuclease sites). A “recombinant vector” indicates that the nucleotide sequence or arrangement of its parts is not a native configuration, and has been manipulated by molecular biological techniques. The term implies that the vector is comprised of segments of DNA that have been artificially joined.

The term “expression vector” as used herein refers to a recombinant DNA molecule containing a desired coding sequence and operably linked nucleic acid sequences necessary for the expression of the operably linked coding sequence in a particular host organism (e.g., a bacterial expression vector, a yeast expression vector or a mammalian expression vector). Nucleic acid sequences necessary for expression in prokaryotes typically include a promoter, an operator (optional), and a ribosome binding site, often along with other sequences. Eukaryotic cells utilize promoters, enhancers, and termination and polyadenylation signals and other sequences which are generally different from those used by prokaryotes.

In some embodiments, eukaryotic expression vectors also contain “viral replicons” or “viral origins of replication.” Viral replicons are viral DNA sequences that allow for the extrachromosomal replication of a vector in a host cell expressing the appropriate replication factors. Some vectors replicate their nucleic acid to high copy numbers (e.g., vectors that contain either the SV40 or polyoma virus origin of replication replicate to high “copy number” (up to 10⁴ copies/cell) in cells that express the appropriate viral T antigen). Other vectors replicate their nucleic acid in low copy numbers (e.g., vectors that contain the replicons from bovine papillomavirus or Epstein-Barr virus replicate extrachromosomally at “low copy number” (˜100 copies/cell). The viral origins of replication listed above are not limiting, as the art is aware of other origins of replication that are commonly used in eukaryotic expression vectors.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and in some embodiments, include gene sequences found in that animal so long as the introduced gene does not reside in the same location as does the naturally-occurring gene.

The terms “overexpression” and “overexpressing” and grammatical equivalents are used in reference to levels of mRNA or protein where the level of expression of the mRNA or protein is higher than that typically observed in a given tissue in a control or non-transgenic animal. Levels of mRNA or protein are measured using any of a number of techniques known to those skilled in the art. For example, in some embodiments mRNA levels are assayed using methods such as Northern blot analysis (however, it is not intended that the present invention be limited to Northern analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample is used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into cells. Transfection can be accomplished by a variety of means known to the art including calcium phosphate (CaPO₄) precipitation, DEAE-dextran-mediated transfection, electroporation, microinjection, lipofection, retroviral infection, microinjection including biolistics and protoplast fusion. Mammalian cell transfection techniques are common in the art, and are described in many sources (See, e.g., Ausubel et al. (eds.), Current Protocols in Molecular Biology, Vol. 1-4, John Wiley & Sons, Inc., New York [1994]).

The term “stable transfection” or “stably transfected” refers to the long term (greater than 3 days post transfection) maintenance and expression of exogenous DNA by a transfected cell. While this term typically encompasses the introduction and integration of foreign DNA into the genome of the transfected cell, it also encompasses the episomal propagation of foreign DNA.

The term “transient transfection” or “transiently transfected” refers to the short term (up to 72 hours post transfection) maintenance and expression of exogenous DNA by a transfected cell. The transfected DNA fails to integrate into the host cell genome and in the absence of selective pressure, the exogenous DNA is lost from the host cell.

The term “calcium phosphate (CaPO₄) precipitation,” refers to a technique for the introduction of nucleic acids into a eukaryotic cell, most typically mammalian cells. The uptake of nucleic acids by cells is enhanced when the nucleic acid is presented as a calcium phosphate-nucleic acid co-precipitate. Various modifications of the original technique of Graham and van der Eb (Graham and van der Eb, Virol., 52:456 [1973]) are known in which the conditions for the transfection of a particular cell type has been optimized. The art is well aware of these various methods.

The term “transformation” has various meanings, depending on its usage. In one sense, the term “transformation” is used to describe the process of introduction of foreign DNA into prokaryotic cells (i.e., bacterial cells), and most frequently E. coli strains. Bacterial cell transformation can be accomplished by a variety of means well known in the art, including the preparation of “competent” bacteria by the use of calcium chloride, magnesium chloride or rubidium chloride, and electroporation. When a plasmid is used as the transformation vector, the plasmid typically contains a gene conferring drug resistance, such as the genes encoding ampicillin, tetracycline or kanamycin resistance. Bacterial transformation techniques are common in the art, and are described in many sources (e.g., Cohen et al., Proc. Natl. Acad. Sci. USA 69: 2110-2114 [1972]; Hanahan, J. Mol. Biol., 166:557-580 [1983]; Sambrook et al. (eds.), Molecular Cloning: A Laboratory Manual, Second Edition, Volumes 1-3, Cold Spring Harbor Laboratory Press, NY, [1989]; Ausubel et al. (eds.), Current Protocols in Molecular Biology, Vol. 1-4, John Wiley & Sons, Inc., New York [1994]).

“Transformation” also describes the physiological process by which a normal eukaryotic cell acquires the phenotypic qualities of a malignant cell. Such properties include, but are not limited to immortality, the ability to grow in soft agar, the ability to grow in nutrient poor conditions, rapid proliferation, and the loss of contact inhibition. A eukaryotic cell which is “transformed” displays the properties of malignant cells. In some embodiments, eukaryotic cells acquire their transformed phenotype in vivo, while in other embodiments, the cells are artificially transformed in culture.

As used herein, the term “selectable marker” refers to the use of a gene that encodes an enzymatic activity that confers the ability to grow in medium lacking what would otherwise be an essential nutrient (e.g., the HIS3 gene in yeast cells); in addition, in some embodiments, a selectable marker confers resistance to an antibiotic or drug upon the cell in which the selectable marker is expressed. Furthermore, some selectable markers are “dominant.” Dominant selectable markers encode an enzymatic activity that is detectable in any suitable eukaryotic cell line. Examples of dominant selectable markers include the bacterial aminoglycoside 3′ phosphotransferase gene (i.e., the neo gene) that confers resistance to the drug G-418 in mammalian cells, as well as the bacterial hygromycin G phosphotransferase (hyg) gene that confers resistance to the antibiotic hygromycin, and the bacterial xanthine-guanine phosphoribosyl transferase gene (i.e., the gpt gene) that confers the ability to grow in the presence of mycophenolic acid. The use of non-dominant selectable markers must be in conjunction with a cell line that lacks the relevant enzyme activity. Examples of non-dominant selectable markers include the thymidine kinase (tk) gene (used in conjunction with tk- cell lines), the carbamoyl-phosphate synthetase, aspartate carbamoyltransferase, and dihydroorotase (CAD) gene (used in conjunction with CAD-deficient cells) and the mammalian hypoxanthine-guanine phosphoribosyl transferase (hprt) gene (used in conjunction with hprt cell lines). A review of the use of selectable markers in mammalian cell lines is provided in Sambrook et al., Molecular Cloning: A Laboratory Manual, Second Edition, Cold Spring Harbor Laboratory Press, New York (1989), at pp.16.9-16.15.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the terms “host,” “expression host,” and “transformant” refer to organisms and/or cells which harbor an exogenous DNA sequence (e.g., via transfection), an expression vector or vehicle, as well as organisms and/or cells that are suitable for use in expressing a recombinant gene or protein. It is not intended that the present invention be limited to any particular type of cell or organism. Indeed, it is contemplated that any suitable organism and/or cell will find use in the present invention as a host.

As used herein, the term “eukaryote” refers to organisms distinguishable from “prokaryotes.” It is intended that the term encompass all organisms with cells that exhibit the usual characteristics of eukaryotes such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “antibody” (or “antibodies”) refers to any immunoglobulin that binds specifically to an antigenic determinant, and specifically, binds to proteins identical or structurally related to the antigenic determinant which stimulated their production. Thus, antibodies are useful in methods to detect the antigen which stimulated their production. Monoclonal antibodies are derived from a single B lymphocyte clone and are generally homogeneous in structure and antigen specificity. Polyclonal antibodies originate from many different clones of antibody-producing cells, and thus are heterogenous in their structure and epitope specificity, but are generally enriched in antibodies which bind to same antigen. In some embodiments, purified monoclonal and/or polyclonal antibodies are used, while in other embodiments, crude preparations are used. For example, in some embodiments, polyclonal antibodies in crude antiserum are utilized. It is intended that the term “antibody” encompass any immunoglobulin (e.g., IgG, IgM, IgA, IgE, IgD, etc.) obtained from any source (e.g., humans, rodents, lagomorphs, non-human primates, caprines, bovines, equines, ovines, etc.).

As used herein, the term “antigen” is used in reference to any substance that is capable of being recognized by an antibody. It is intended that this term encompass any antigen and “immunogen” (i.e., a substance which induces the formation of antibodies). Thus, in an immunogenic reaction, antibodies are produced in response to the presence of an antigen or portion of an antigen. The terms “antigen” and “immunogen” are used to refer to an individual macromolecule or to a homogeneous or heterogeneous population of antigenic macromolecules. It is intended that the terms antigen and immunogen encompass protein molecules or portions of protein molecules, which contains one or more epitopes. In many cases, antigens are also immunogens, thus the term “antigen” is often used interchangeably with the term “immunogen.” An immunogenic substance can be used as an antigen in an assay to detect the presence of appropriate antibodies in the serum of the immunized animal.

As used herein, the terms “antigen fragment” and “portion of an antigen” and the like are used in reference to a portion of an antigen. Antigen fragments or portions occur in various sizes, ranging from a small percentage of the entire antigen to a large percentage (less than 100%) of the antigen. However, in situations where at least a portion of an antigen is specified, it is contemplated that the entire antigen is also present (although it is not required that the entire antigen be present). In some embodiments, antigen fragments and/or portions do not comprise an “epitope” recognized by an antibody, while in preferred embodiments, antigen fragments and/or portions do comprise an epitope that is recognized by an antibody (e.g., an antibody of interest). In some embodiments, antigen fragments and/or portions are not immunogenic, while in preferred embodiments, antigen fragments and/or portions are immunogenic.

The terms “antigenic determinant” and “epitope” as used herein refer to that portion of an antigen that makes contact with a particular antibody variable region. When a protein or fragment (or portion) of a protein is used to immunize a host animal, numerous regions of the protein may induce the production of antibodies which bind specifically to a given region or three-dimensional structure on the protein (i.e., these regions or structures are referred to as antigenic determinants). In some embodiments, an antigenic determinant (e.g., a fragment of an antigen) competes with the intact antigen (i.e., the “immunogen” used to elicit the immune response) for binding to an antibody.

The terms “specific binding” and “specifically binding” when used in reference to the interaction between an antibody and an antigen describe an interaction that is dependent upon the presence of a particular structure (i.e., the antigenic determinant or epitope) on the antigen. In other words, the antibody recognizes and binds to a protein structure unique to the antigen, rather than binding to all proteins in general (i.e., non-specific binding).

As used herein the term “immunogenically-effective amount” refers to that amount of an immunogen required to invoke the production of protective levels of antibodies in a host upon vaccination.

As used herein, the term “adjuvant” is defined as a substance which enhances the immunogenicity of a co-administered antigen. If adjuvant is used, it is not intended that the present invention be limited to any particular type or that the same adjuvant, once used, be used for all subsequent immunizations. The present invention contemplates many adjuvants, including but not limited to agar beads, aluminum hydroxide or phosphate (alum), Freund's adjuvant (incomplete or complete), Quil A adjuvant and Gerbu adjuvant (Accurate Chemical and Scientific Corporation), and bacterins (i.e., killed preparations of bacterial cells, especially mycoplasma).

As used herein, the term “immunoassay” refers to any assay that uses at least one specific antibody for the detection or quantitation of an antigen. Immunoassays include, but are not limited to, Western blots, enzyme-linked immunosorbent assays (ELISAs or EIAs), radioimmunoassays (RIAs), and immunofluorescence assays (IFAs). Furthermore, many different ELISA formats are known to those in the art, and which find use in the present invention. However, it is not intended that the present invention be limited to these assays. Thus, other antigen-antibody reactions find use in the present invention, including but not limited to “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen), “complement fixation” (i.e., the use of complement in an antibody-antigen reaction method), and other methods commonly used in serology, immunology, immunocytochemistry, immunohistochemistry, and related fields.

As used herein, the term “ELISA” refers to enzyme-linked immunosorbent assay (or EIA). Numerous ELISA methods and applications are known in the art, and are described in many references (See e.g., Crowther, “Enzyme-Linked Immunosorbent Assay (ELISA),” in Molecular Biomethods Handbook, Rapley et al [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]).

One ELISA method finding use with the present invention is a “direct ELISA.” In this embodiment, an antigen is immobilized to a solid support (e.g., a microtiter plate well), and is detected directly using an enzyme-conjugated antibody specific for the antigen. In an alternative embodiment, an “indirect ELISA” is used. In this embodiment, an antigen is immobilized to a solid support (e.g., a microtiter plate well) as in the direct ELISA, but is detected indirectly by first adding an antigen-specific antibody, followed by the addition of a detection antibody specific for the antibody that specifically binds the antigen. These secondary antibodies can be “species-specified ” antibodies (e.g., a goat anti-rabbit antibody), which are commercially available from a variety of vendors (e.g., Santa Cruz Biotechnology; Zymed; and Pharmingen/Transduction Laboratories).

“Sandwich ELISAs” also find use with the present invention. In a sandwich ELISA, the antigen is immobilized on a solid support (e.g., a microtiter plate) via an immobilized antibody (i.e., a capture antibody) which binds the antigen of interest. Following the affixing of a suitable capture antibody, a sample is added to the microtiter plate well, followed by washing. If the antigen of interest is present in the sample, it is bound to the capture antibody present on the support. In some embodiments, the sandwich ELISA is a “direct sandwich” ELISA, in which the captured antigen is detected directly by using an enzyme-conjugated antibody also directed against the antigen of interest. In alternative embodiments, the sandwich ELISA is an “indirect sandwich” ELISA, in which the captured antigen is detected indirectly by using an antibody directed against the antigen, which is then detected by another antibody which is enzyme-conjugated, thus forming an antibody-antigen-antibody-antibody complex. Suitable reporter reagents are then added to detect the third antibody. Alternatively, in other embodiments, any number of additional antibodies are added as necessary to detect the antigen-antibody complex. In some embodiments, these additional antibodies are also labelled or tagged to permit their visualization and/or quantitation.

As used herein, the term “capture antibody” refers to an antibody that is used in a sandwich ELISA (or other “sandwich” type immunoassays) to bind (i.e., capture) an antigen in a sample prior to detection of the antigen. Biotinylated capture antibodies are typically used in the present invention in conjunction with avidin-coated solid supports. Another antibody (i.e., the detection antibody) is then used to bind and detect the antigen-antibody complex, in effect forming a “sandwich” comprised of antibody-antigen-antibody (i.e., a sandwich ELISA).

As used herein, a “detection antibody” is an antibody which carries on it a means for visualization or quantitation, which is typically a conjugated enzyme moiety that yields a colored or fluorescent reaction product following the addition of a suitable substrate. Conjugated enzymes commonly used with detection antibodies in ELISAs include horseradish peroxidase, urease, alkaline phosphatase, glucoamylase and β-galactosidase. In some embodiments, detection antibodies are directed against the antigen of interest, while in other embodiments, they are not. Typically, detection antibodies are anti-species antibodies. Alternatively, the detection antibody is prepared with a label such as biotin, a fluorescent marker, or a radioisotope, and is detected and/or quantitated using this label.

As used herein, the term “reporter reagent” or “reporter molecule” or “detection substrate” or “detection reagent” is used in reference to reagents which permit the detection and/or quantitation of an antibody bound to an antigen. For example, in preferred embodiments, a reporter reagent is a calorimetric substrate for an enzyme that has been conjugated to an antibody. A suitable substrate in the presence of the antibody-enzyme conjugate results in the production of a calorimetric or fluorimetric signal. Other reporter reagents include, but are not limited to, radioactive compounds. This definition also encompasses the use of biotin and avidin-based compounds (e.g., including but not limited to neutravidin and streptavidin) as part of the detection system.

As used herein, the term “signal” is used generally in reference to any detectable process that indicates that a reaction has occurred, for example, binding of antibody to antigen. It is contemplated that signals in the form of radioactivity, fluorimetric or calorimetric products/reagents find use with the present invention. In some embodiments, the signal is assessed quantitatively, while in other embodiments, the signal is assessed qualitatively (or both quantitatively and qualitatively).

As used herein, the term “amplifier” is used in reference to a system which enhances the signal in a detection method, such as an ELISA (e.g., an alkaline phosphatase amplifier system used in an ELISA).

As used herein, the term “solid support” is used in reference to any solid material to which reagents such as antibodies, antigens, and other components may be attached. For example, in the ELISA method, the wells of microtiter plates provide solid supports. Other examples of solid supports include microscope slides, coverslips, beads, particles, cell culture flasks, as well as many other suitable items.

As used herein, the term “kit” is used in reference to a combination of reagents and other materials which facilitates an assay and the analysis of a sample. In some embodiments, the immunoassay kits of the present invention include suitable capture antibody, reporter antibody, antigen, detection reagents and an amplifier system. Furthermore, in some embodiments, the kit also includes, but is not limited to, apparatus for sample collection including sample tubes, holders, trays, racks, dishes, plates, instructions to the kit user, solutions or other chemical reagents, and samples to be used for standardization, normalization, and/or positive and negative controls.

The terms “Western blot,” “Western immunoblot” “immunoblot” and “Western” refer to the immunological analysis of protein(s), polypeptides or peptides that have been immobilized onto a membrane support. The proteins are first resolved by sodium dodecyl sulfate-polyacrylamide gel electrophoresis (i.e., SDS-PAGE) to separate the proteins, followed by transfer of the proteins from the gel to a solid support, such as nitrocellulose, polyvinylidene difluoride (PVDF) or a nylon membrane. The immobilized proteins are then exposed to an antibody having reactivity towards an antigen of interest. The binding of the antibody (i.e., the primary antibody) is detected by use of a secondary antibody which specifically binds the primary antibody. The secondary antibody is typically conjugated to an enzyme which permits visualization of the antigen-antibody complex by the production of a colored reaction product or the catalysis a luminescent enzymatic reaction (e.g., the ECL reagent, Amersham).

The term “sample” as used herein is used in its broadest sense. The term “sample” as used herein is typically of biological origin, where “sample” refers to any type of material obtained from animals or plants (e.g., any fluid or tissue), cultured cells or tissues, cultures of microorganisms (prokaryotic or eukaryotic), and any fraction or products produced from a living (or once living) culture or cells. A sample may be a cell extract (i.e., a crude lysate), and can be purified or unpurified. Samples suspected of containing a nucleic acid encoding a Rig protein encompass (i.e., are not limited to) a cell or cells, chromosomes or nucleic acid isolated from a cell, genomic DNA (in solution or bound to a solid support such as for Southern blot analysis), RNA (in solution or bound to a solid support such as for Northern blot analysis), cDNA (in solution or bound to a solid support), synthetic nucleic acids, and the like. A sample suspected of containing a Rig protein can comprise, but is not limited to, a cell, a portion of a tissue (e.g., a biopsy specimen), and/or an extract containing one or more proteins and the like. An “experimental sample” is a sample where the presence, concentration and/or activity of some molecule of interest is unknown. A “control sample” is a sample where the presence, concentration and/or activity of some molecule of interest is known. For example, control samples containing known concentrations of some molecule of interest (e.g., purified Rig protein), can be used to determine the concentration of Rig protein in an experimental sample by using a suitable assay (e.g., a Rig-specific ELISA) to construct a standard concentration curve. As used herein, a sample “suspected of containing” a component or biological/biochemical activity is a sample where the presence of the component or activity has not been demonstrated or proven.

As used herein, the term “host cell” refers to any cell capable of harboring an exogenous nucleic acid or gene product. In some embodiments, the host cell also transcribes and/or translates and expresses a gene contained on the exogenous nucleic acid. It is intended that the exogenous nucleic acid be obtained from any suitable source. In some embodiments, it is produced synthetically, while in other embodiments, it is produced by another cell or organism. In addition, in some embodiments, the exogenous nucleic acid is subjected to replication, while in other embodiments, it is not. For example, the bacterium Escherichia coli is suitable for use as a host cell for a bacterial expression vector encoding the Rig polypeptide.

As used herein, the term “in vitro” refers to an artificial environment and to processes or reactions that occur within an artificial environment. The term “in vivo” refers to the natural environment (e.g., in an animal or in a cell) and to processes or reactions that occur within a natural environment. The definition of an in vitro versus an in vivo system is particular for the system under study.

As used herein, the term “subject” refers to any animal being examined, studied or treated. It is not intended that the present invention be limited to any particular type of subject. It is contemplated that multiple organisms will find use in the present invention as subjects. In some embodiments, humans are the preferred subject.

As used herein, the term “inhibit” refers to the act of diminishing, suppressing, alleviating, preventing, reducing or eliminating. For example, in some embodiments, a method that inhibits cell growth results in the prevention of further cell growth, slows cell growth, or kills cells. The term “inhibit” applies equally to both in vitro and in vivo systems.

As used herein, the term “DNA-dependent DNA polymerase” refers to a DNA polymerase that uses a single strand of deoxyribonucleic acid (DNA) as a template for the synthesis of a complementary and antiparallel DNA strand.

As used herein, the term “RNA-dependent DNA polymerase” refers to a DNA polymerase that uses ribonucleic acid (RNA) as a template for the synthesis of a complementary and antiparallel DNA strand. The process of generating a DNA copy of an RNA molecule is commonly termed “reverse transcription,” and the enzyme that accomplishes this is a “reverse transcriptase” (RT). In some cases, a reverse transcriptase also contains ribonuclease activity. Furthermore, some DNA polymerase enzymes contain both DNA-dependent as well as RNA-dependent DNA polymerase activity. These dual-activity polymerases are frequently used in RT-PCR reactions.

As used herein, a “thermostable” enzyme is, in its most general sense, an enzyme that retains activity at elevated temperatures. In some embodiments, a thermostable DNA-polymerase, as used in PCR reactions, retains polymerase activity at temperatures at or in excess of 90° C. However, it is not intended that the present invention be limited to thermostable enzymes with a specific range of activity. Rather, it is intended that the term encompasses enzymes that are active at temperatures that are higher that the optimum temperature of mesophilic enzymes.

As used herein, the term “tumor” refers to a neoplasia, and most frequently, to a malignant neoplasia.

As used herein, a “solid tumor” is a tumor that forms a mass with defined borders. As used herein, “tumor tissue” refers to tissue (including cells) from a solid tumor.

As used herein, the term “non-tumorigenic tissue” is tissue (including cells) that is free of tumor, or does not otherwise give rise to tumor tissue.

As used herein, the terms “local” or “localized” and the like refer to confinement to a small area, a single tissue (e.g., ovarian tissue), a single organ (e.g., a lung) or other structure (e.g., a solid tumor).

As used herein, the term “localized delivery” is delivery of an agent (e.g., a gene therapy agent or a drug) to a small area, a single tissue, a single organ or other specific structure (e.g., a solid tumor). For example, localized delivery of a gene therapy agent to a single site (e.g., a solid tumor) in a subject is typically achieved by injection into that site.

As used herein, the term “systemic” refers to multiple sites, tissues or organs in an organism, or to the entire organism. Use of the word “systemic” generally indicates involvement of the circulatory or lymphatic systems.

As used herein, the term “systemic delivery” (in contrast to localized delivery) is delivery of an agent (e.g., a drug) to multiple sites, tissues or organs in an organism, or to the entire organism via the circulatory system following an intravenous injection, or via gastrointestinal absorption of an orally administered agent.

As used herein, the term “surgical delivery” refers to the delivery of an agent (e.g., a gene therapy agent) by surgical means (i.e., by operation or some other invasive manipulation). Thus, in some embodiments, surgical techniques provide means for localized delivery of an agent.

As used herein, the terms “implant” or “implantation” or the like refer to the grafting or insertion of some device or structure into an organism. As used herein, a device (e.g., a capsule or chamber) for controlled or extended release of a therapeutic agent (e.g., a gene therapy agent) is implanted into a subject. The implantation of devices for the delivery of therapeutic agents offers the benefit of delivery to a localized area (i.e., not systemically), increased localized concentration of the agent, as well as extended and continuous release of the agent to the localized area.

### DETAILED DESCRIPTION OF THE INVENTION

Ras proteins have been most extensively studied for their role in cell proliferation and tumorigenesis. However, a paradoxical observation regarding the function of Ras has emerged. Ras proteins are not only involved in signaling pathways which control cell proliferation, but they also transduce signals which result in growth inhibition, growth arrest and/or apoptosis. Examples of this phenomenon are demonstrated in a variety of cellular systems, and include the ability of Ras to induce senescence (Serrano et al., Cell 88:593-602 [1997]), necrosis (Chi et al., Oncogene 18:2281-2290 [1999]), apoptosis (Mayo et al., Science 278:1812-1815 [1997]; Chen and Faller, Oncogene 11:1487-1498 [1995]; and Joneson and Bar-Sagi, Mol. Cell. Biol., 19:5892-5901 [1999]), and terminal differentiation (Bar-Sagi and Feramisco, Cell 42:841-848 [1985]). The signalling mechanisms behind Ras and Ras-family mediated growth inhibition and apoptosis remain poorly understood. However, an understanding of the mechanism(s) is not necessary in order to use the present invention, nor is it intended that the present invention be limited to any particular mechanism(s).

The present invention provides means to identify Ras-superfamily genes which function in growth inhibition, and/or have tumor suppressor properties. Genes which fit this criteria are excellent candidates for development as anti-cancer therapeutics in the treatment of cancers which display elevated Ras activity.

For convenience, the remainder of the Detailed Description of the Invention is divided into the following sections:

I. Identification, Isolation, Subcloning and Sequencing of the Ras-Family Gene and Protein Rig;

II. Analysis of Rig mRNA Expression;

III. Rig Inhibits Cell Growth and Ras Signaling Activity

IV. Rig Interacts with Raf-1

V. Generation of Anti-Rig Antibodies

VI. Analysis of Rig Protein Expression

VII. Analysis of Rig Prenylation

VIII. Analysis of Rig Subcellular Localization

IX. Compositions and Methods for the Detection of the Rig Gene, Transcript and Protein

X. Methods for Inhibiting Cell Growth

XI. Pharmaceutical Compositions Comprising the Rig Gene for the Treatment of Cancer

I. Identification, Isolation, Subcloning and Sequencing of the Ras-Family Gene and Protein Rig

In order to identify novel Ras-family gene products which have tumor suppressor activity, an electronic screen was undertaken to identify proteins showing homology to the Noey2 (also called Ahri1) gene product (Yu et al., Proc. Natl. Acad. Sci. USA 96:214-219 [1999]; GenBank Accession No. NP_{—}004666; and SEQ ID NO:9). The full length Noey2 amino acid sequence (SEQ ID NO:9) was used as the query sequence to search the databases of the National Center for Biotechnology Information (NCBI), using the search program “Advanced tBLASTn” (>http://www.ncbi.nlm.nih.gov/BLAST/<). This electronic screen identified an open reading frame (ORF) on bacterial artificial chromosome (BAC) 41195 (GenBank Accession No. AC006538), corresponding to a region on human chromosome 19 at p13.3. This ORF was then used to search the NCBI expressed sequence tag (EST) databases. That search identified an IMAGE Consortium EST (IMAGE Consortium Clone ID No. 2165313; GenBank Accession No. AI49781 1) containing a human ORF corresponding to the region of interest on the BAC 41195 clone. This IMAGE Consortium Clone was obtained from ATCC (Catalog No. 3363561) and used as template material in a PCR strategy to isolate the full length ORF. These primers contained terminal BamHI and EcoRI restriction sites, respectively, to facilitate subcloning. The resulting PCR product was subcloned into a variety of vectors for subsequent study.

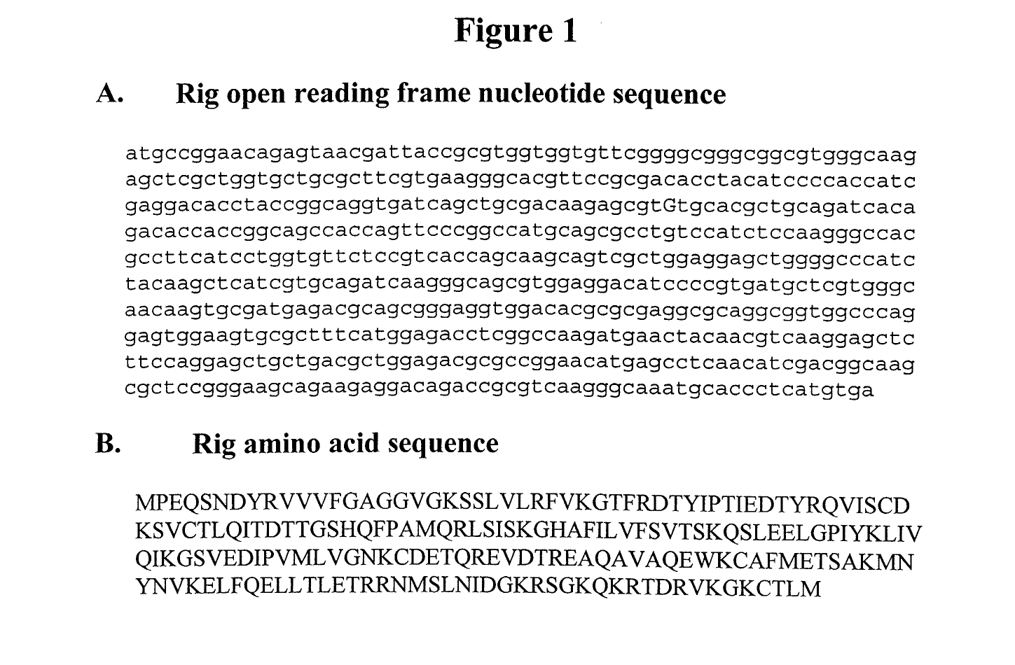

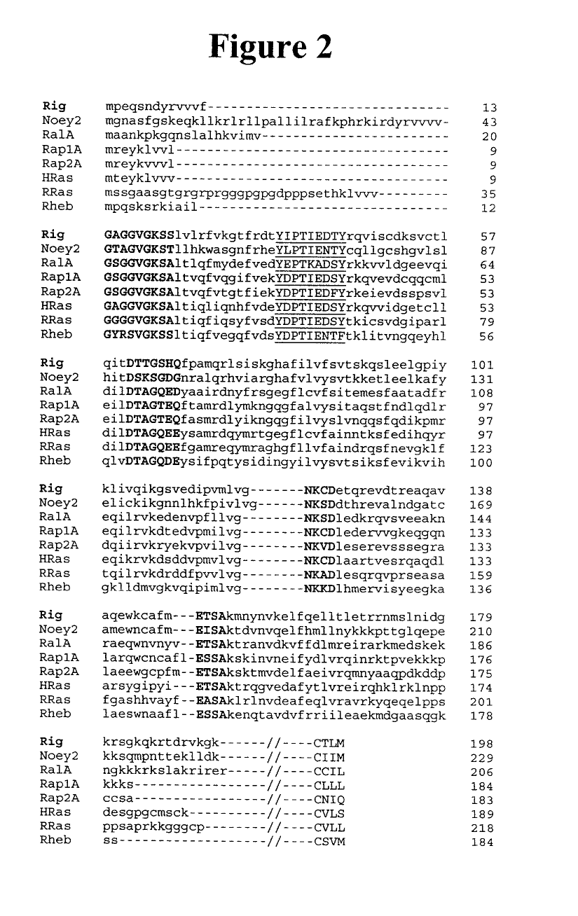

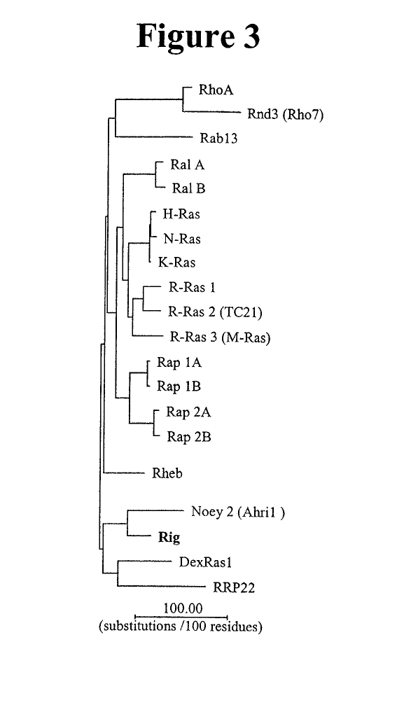

This PCR strategy identified a 597 base pair human ORF, including the 3′ stop codon; shown in FIG. 1, Panel A; and SEQ ID NO:4). This ORF (i.e., gene and polypeptide) are herein called “Rig.” The Rig ORF was predicted to encode a 198 amino acid polypeptide (shown in FIG. 1, Panel B; GenBank Accession No. AAD13119; and SEQ ID NO:5). The polypeptide encoded by this ORF was compared to other Ras-family members using PileUp and GrowTree programs of the Genetics Computer Group (GCG) suite of sequence analysis tools. Sequence alignment and dendogram comparison of the Rig polypeptide with other Ras-family proteins is shown in FIGS. 2 and 3, respectively.

From these comparisons, it was determined that the Rig protein shares significant homology with a large number of Ras-family monomeric GTP-binding proteins. Rig shares strongest homology to the Noey2 gene product (SEQ ID NO:9; and GenBank Accession No: NP_{—}004666), where the two proteins share 63% sequence similarity over their full length, 71% identity in the guanine nucleotide-binding domain and 77% identity in the effector domains. The Rig protein contains a C-terminal CAAX (SEQ ID NO:1) motif located at amino acid positions 195-198, which serves as a signals for post-translational prenylation. Surprisingly, the Rig protein contains a serine at amino acid residue 65, suggesting that the Rig protein does not have GTPase activity. In FIG. 2, the Rig protein guanine nucleotide binding domain is indicated in bold capital letters, the effector binding domain is indicated by underlined capital letters, and the CAAX (SEQ ID NO:1) motif is simply capitalized.

II. Analysis of Rig mRNA Expression

The present invention provides compositions and methods for the analysis of Rig RNA expression. In preferred embodiments, these methods use Northern blotting techniques to examine Rig RNA expression in a variety of tissues and cell lines (e.g., human, mouse, etc.), using both electrophoretic and slot-blot formats. A probe corresponding to the 597 base pair Rig ORF produced by random-primed ³²P-dCTP labelling was used in these Northern blots.

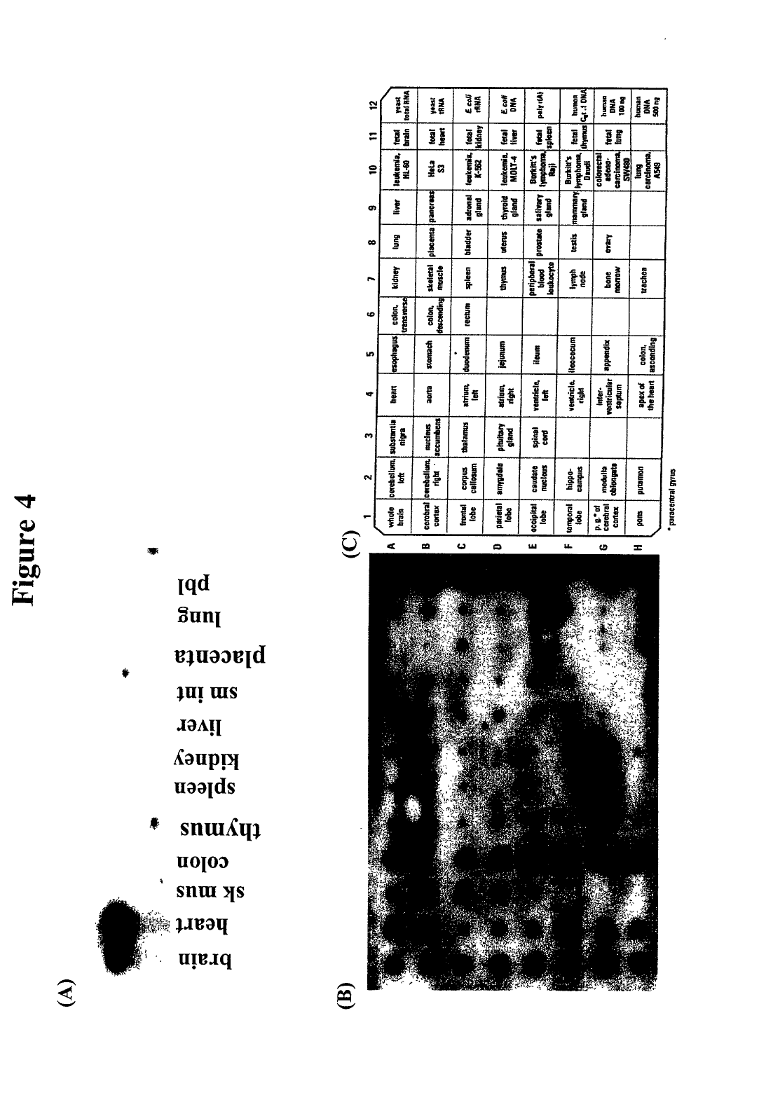

In one experiment, Rig mRNA expression was analyzed using a multiple human tissue poly(A)⁺ mRNA Northern blot (Clontech) probed with a Rig cDNA probe. As shown in FIG. 4, Panel A, a single predominant transcript corresponding to the Rig gene was present in the poly(A)⁺ mRNA derived from brain and heart. No Rig expression was observed in skeletal muscle, colon, thymus, spleen, kidney, liver, small intestine, placenta, lung or peripheral blood leukocytes.

In another experiment, a multiple human tissue poly(A)⁺ mRNA Northern slot blot (Clontech) was probed with the Rig ORF probe. FIG. 4, Panel C shows the key to the RNA samples in the slot blot. As shown in FIG. 4, Panel B, strong positive signals indicative of Rig message were detected in most adult brain and cardiac structures, as well as fetal brain and heart tissue. Significantly weaker signals were also observed in some additional adult tissues.

In another experiment, the expression pattern of the Rig gene was analyzed in tumor-derived neural cell lines by Northern blotting. Total RNA was isolated from cultured U251 (a human glioblastoma), TC32 (a human Ewing sarcoma), TC106 (a human PNET—primitive neuroectodermal tumor), A673 (human rhabdomyosarcoma/undifferentiated carcinoma; ATCC CRL No. 7910), SH5Y (a human neuroblastoma) and NE115 (a murine neuroblastoma) cell lines. Samples of the total RNA were resolved by agarose-formaldehyde gel electrophoresis, transferred to nylon membranes, and probed with the Rig ORF probe.

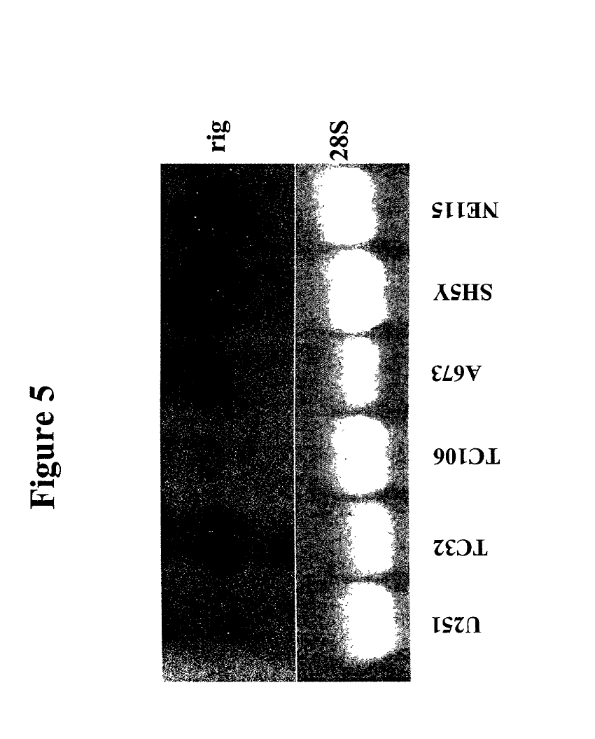

As shown in FIG. 5, virtually no Rig mRNA was detected in the U251 and TC106 cell lines, whereas Rig expression varied in the other cell lines. The lower panel in FIG. 5 shows loading-normalization staining of 28S ribosomal RNA from the same blot. No genomic deletions of the Rig gene were found, as determined by Southern blotting analysis (data not shown).

Thus, strong Rig expression is observed in heart and brain tissues, and is absent in some tumor cell lines. Therefore, the present invention provides compositions and methods for the detection of Rig transcripts in cells and tissues, using both polyA⁺ and total RNA as sample materials. However, it is not intended that the present invention be limited to the particular reagents for RNA isolation and Northern blotting described herein, as one familiar with the art knows numerous equivalent reagents that also find use with the present invention. Furthermore, it is also not intended that analysis of Rig expression be limited to human or mouse cells, nor to the tissues or cell types itemized herein, as the present invention finds use in the analysis of Rig expression in numerous cells, tissues and species.

III. Rig Inhibits Cell Growth and Ras Signaling Activity

A variety of experiments were undertaken to determine Rig function, and more specifically, to examine the role of Rig on the regulation of cell growth and Ras signaling. Thus, the present invention provides means for the assessment of Rig biological activity.

In one experiment, a clonogenic focus formation assay was used to observe the effect of Rig expression on cell growth. This assay involved the stable introduction of expression vectors encoding wild-type or mutant Rig proteins into NIH-3T3 mouse fibroblast cells, and subsequently observing the ability of the transfected cells to form clonogenic colonies. The expression vectors constructed for use in this experiment were based on the pZIP-NeoSV(x)1 retroviral-based expression vector, which carries the gene encoding neomycin resistance (neo). This vector programs constitutive recombinant protein production by placing the ORF under the control of the Moloney murine leukemia virus (MoMuLV) LTR promoter. Furthermore, this vector contains an upstream hemagglutinin (HA) tag resulting in the synthesis of HA-tagged fusion proteins. A pZIP-Neo(HA) vector encoding a mutant Rig(S17N) protein was produced using oligonucleotide site-directed mutagenesis. The Rig(S21N) mutant protein is analogous to the Ras(S17N) mutation, which acts in a dominant-negative manner by abolishing GTP-binding.

NIH-3T3 cells were transfected with the recombinant expression vectors by the calcium phosphate (CaPO₄) precipitation method known in the art. Following transfection, stably transfected NIH-3T3 cells were selected using the neomycin-analog G418/GENTICIN® (GIBco/Life Technologies), and surviving cells were maintained on the same plate in culture and allowed to form clonal cell colonies. After 10-14 days, cells were stained with crystal violet to facilitate observation and colony counting. This experiment was performed in duplicate at least 3 times.

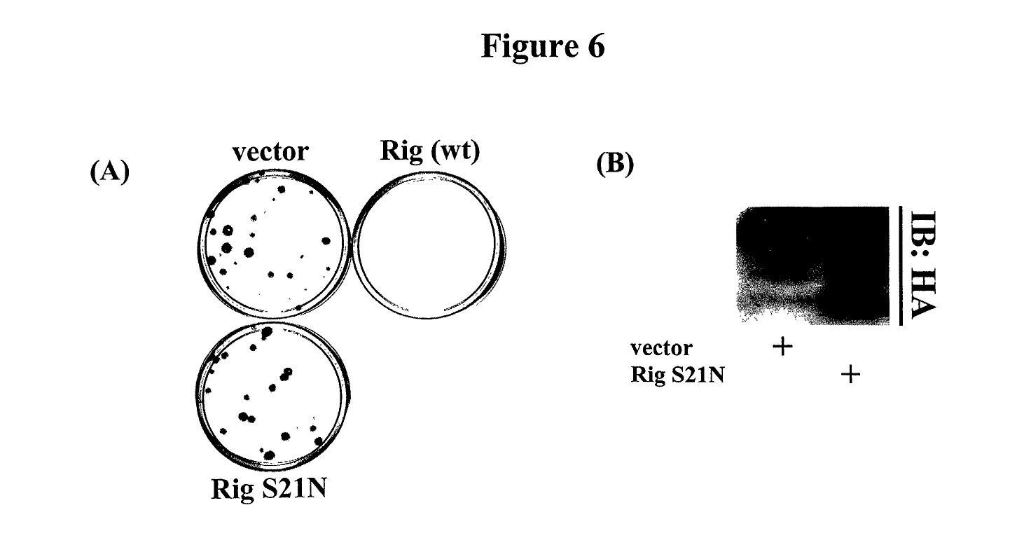

Representative images of the transfected plates are shown in FIG. 6, Panel A. As shown in the Figure, cells transfected with the empty expression vector pZIP-Neo (HA) (vector) formed abundant neo resistant colonies. However, cells transfected with wild-type Rig did not survive G418 selection. Cells transfected with the dominant-negative form of Rig also showed abundant colony formation following G418 selection. Thus, when stably expressed, Rig inhibits or prevents cell growth. Similar results were observed using COS-7 (SV-40 transformed, African Green Monkey kidney cell line; ATCC CRL No. 1651), HEK-293T (a transformed human embryonal kidney cell line, ATCC CRL No. 1573), TC32 (a human Ewing sarcoma), A673 (human rhabdomyosarcoma/undifferentiated carcinoma; ATCC CRL No. 7910), U251 (a human glioblastoma), TC106 (a human PNET—primitive neuroectodermal tumor) and SH5Y (a human neuroblastoma) cell lines (data not shown). It was for this reason that this gene and protein were called “Rig,” for Ras-related Inhibitor of Growth.

FIG. 6, Panel B, shows immunoblotting results of lysates from the stable NIH-3T3 cell lines using an anti-HA primary antibody and a horseradish peroxidase (HRP)-conjugated goat anti-mouse secondary antibody. This blot confirmed expression of the mutant HA-tagged Rig(S21N) protein. Thus the result of the clonogenic colony assay was not the result of deletion or misexpression of the mutant Rig protein.

In another experiment using a similar method, the ability of Rig to inhibit Ras-mediated transformation was examined. This experiment used a transformation/focus formation assay, as known in the art. In this experiment, NIH-3T3 cells were transfected with Ras, Rig and Rap(Q63E) expression vectors using the calcium phosphate (CaPO₄) technique as known in the art. This Rap(Q63E) mutant allele has been previously shown to antagonize Ras transformation (Clark et al., J. Biol. Chem., 272:10608-10615 [1997]). To express the Rig(wt) ORF, this nucleotide sequence was subcloned into the pCDNA3.1 expression vector (Invitrogen), which places the cloned ORF under the strong, constitutively-active CMV promoter. A mammalian pCGN expression vector (Fiordalisi et al., Methods Enzymol., 332:3-36 [2001]), also driven by the CMV promoter region, was used to express the mutant Rap(Q63E) protein.

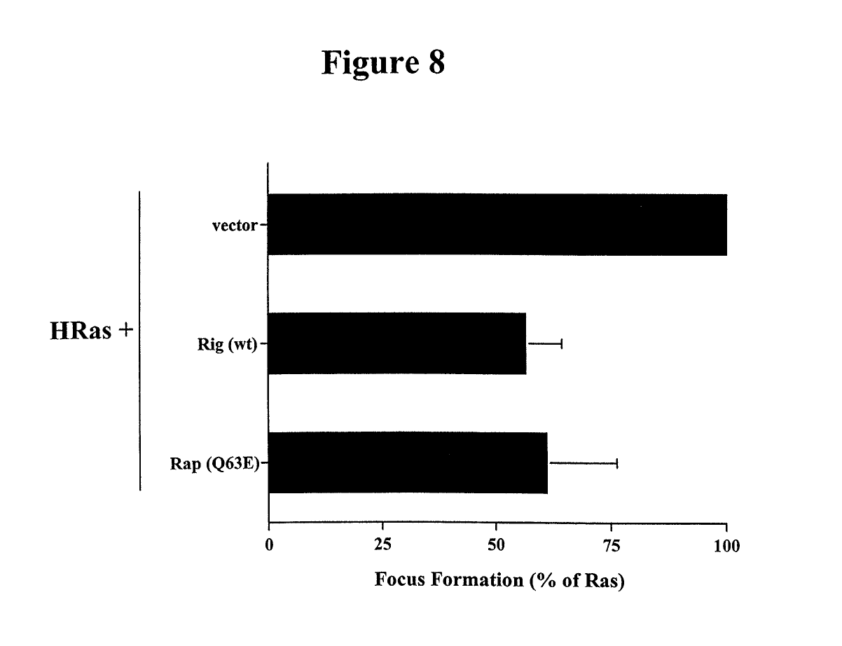

In this experiment, each transfection tube contained a pCGN-Ras(G12V) mammalian expression vector driven by the CMV promoter region (Fiordalisi et al., Methods Enzymol., 332:3-36 [2001]), and either (a) an empty mammalian expression vector, (b) a Rig(wt) expression vector, or (c) a Rap(Q63E) mutant expression vector. Following transfection, the cells were selected for neomycin resistance, and allowed to form transformed foci on the original plate. The foci were visualized using an inverted microscope and scored accordingly. Focus formation values are depicted as percentages of the focus formation with Ras(G12V) transformation without any co-expressed gene. As shown in FIG. 8, co-transfection of Ras(G12V) with Rap(Q63E) inhibited Ras-mediated transformation. Furthermore, co-transfection of the Rig(wt) expression vector with Ras(G12V) also resulted in inhibition of Ras-induced foci formation. Thus, Rig has the ability to inhibit Ras-mediated transformation.

In yet another experiment, the ability of Rig to regulate Ras signalling was examined. This analysis used an Elk-1-dependent luciferase assay to indirectly assess Ras activity following transient expression of Rig. Elk-1 is a downstream transcription factor regulated by Ras signaling. A transient transfection assay was used in this experiment to determine the ability of the Rig protein to regulate transcriptional activity of the Elk-1 transcription factor in the presence of an activated Ras (i.e., in a Ras-dependent manner). The Elk-1 transcription factor is expressed as a fusion protein with the Gal4 DNA binding domain, and transcriptional activation activity of the fusion protein is measured by a luciferase reporter construct containing 5 tandem Gal4 DNA-binding sites (Stratagene). A Rig(wt) expression vector was produced using the pCDNA3.1 expression vector (Invitrogen).

In this transient transfection assay, NIH-3T3 cells were co-transfected using the calcium phosphate (CaPO₄) precipitation method with Gal-Elk-1 expression vector (Stratagene), a Ras(G12V)/pCGN mammalian expression vector, and the Gal-Luciferase reporter plasmid (Stratagene). In addition, cells also received various quantities (10-1000 ng) of the Rig(wt) expression vector. Following the transfection, cells were cultured for 48 hours in culture medium containing 10% calf serum, then shifted to culture medium containing 1% calf serum overnight. The next day, the resulting quiescent transfected cells were harvested in luciferase lysis buffer, and luciferase activity was measured.

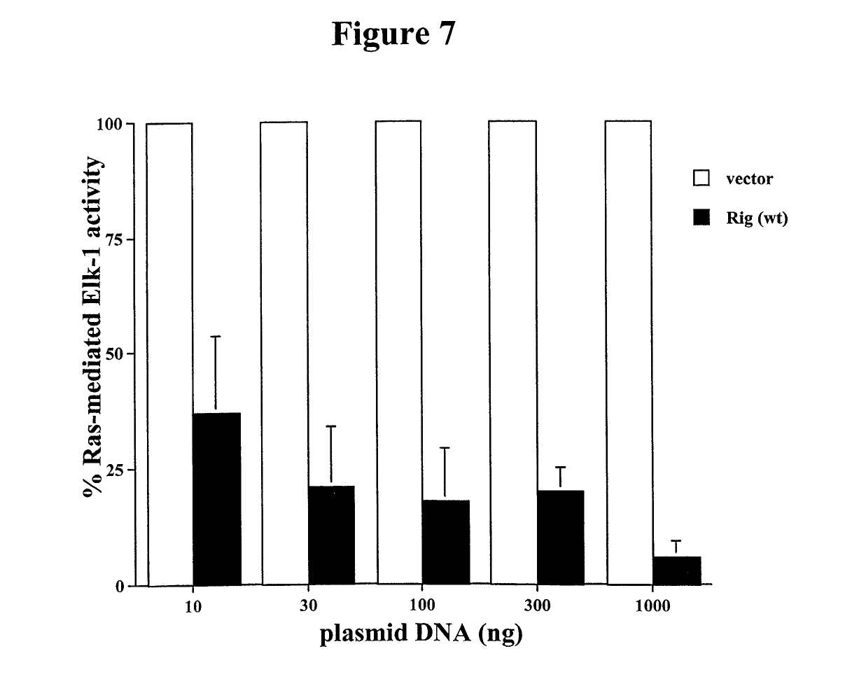

The results of this assay are shown in FIG. 7. Data are represented as percent of Ras-mediated Elk-1 activity, where the observed Elk-1 activity using only Ras without any exogenous Rig has been normalized to 100%. Rig has an antagonistic effect on Ras-dependent Elk-1 activity, even when as little as 10 ng of the Rig(wt) expression vector was introduced. Increasing concentrations of this Rig expression vector showed increasing Elk-1 inhibitory effects. Thus, Rig has an antagonistic effect on Ras signaling.

An experiment was also conducted to observe the effect of Rig expression on the growth rates of neural tumor cell lines. In these experiments the growth rate was monitored growth rate by counting cells following the induced expression of Rig within the U251 glioblastoma and A673 rhabdomyosarcoma tumor cell lines. Due to the inhibitory effect of Rig expression on cell growth and survival, Rig expression in this experiment was accomplished through the use of a recombinant tetracycline-inducible Moloney murine leukemia virus (M-MuLV)-based retroviral vector, pLRT.

The LRT retroviral vector contains retroviral sequences that permit the production of non-replicating, infective virion, in the presence of helper functions provided by a suitable packaging cell line. The LRT vector contains a cloning site and regulatory sequences that facilitate the subcloning and conditional expression of a desired ORF, under the transcriptional control of a tetracycline-sensitive promoter, such that addition of tetracycline (or the tetracycline-analog doxycycline) results in induced transcription and protein expression. In addition, the LRT vector encodes and constitutively expresses the blasticidin-S-deaminase (BSD) gene product, which imparts resistance to the cytotoxic drug blasticidin to cells which harbor the virus.

For safety purposes, variants of the U251 and A673 neural tumor-derived human cell lines were constructed that express the ecotropic docking receptor (Wang et al., Nature 352:729-731 [1991]). To accomplish this, the gene encoding the ecotropic docking receptor was subcloned into the pBabe retroviral vector (Morgenstern et al., Nucl. Acids Res., 18:3587-3596 [1990]), then transfected into the amphotropic packaging cell line PA317 (Kwok et al., Proc. Natl Acad. Sci. USA 83:4552-4555 [1986]), followed by selection of stable transfectants in puromycin. Retroviral-conditioned medium produced by the engineered PA317 cells was used to infect human U251 and A673 neural cells, followed by selection for stably infected recipient cells in puromycin. Thus, these engineered U251 and A673 cells are now capable of infection by virion produced by an ecotropic retroviral producer cell line.

To construct the Rig-LRT vector, a XhoI/NotI fragment containing the Rig ORF and the FLAG epitope tag was cloned into the same sites of the pLRT plasmid, to form pLRT-Rig-FLAG. This pLRT-Rig-FLAG vector was then transfected into the Phoenix ecotropic retroviral packaging cell line (Nolan and Shatzman, Curr. Opin. Biotechnol., 9(5):447-450 [1998]; Hofmann et al., Proc. Natl. Acad. Sci. USA 93(11):5185-5190 [1996]). Infective pLRT-Rig-FLAG virions were collected from the Phoenix culture supernatants, and these supernatants were subsequently used to stably infect the engineered ecotropic receptor-positive U251 and A673 host cells. Following infection, stable integration of the pLRT-Rig-FLAG viral genome was selected for by the addition of blasticidin, permitting the establishment of polyclonal U251(pLRT-Rig-FLAG) and A673(pLRT-Rig-FLAG) cell lines.

Following establishment of the stably infected cell lines, parallel cultures of the U251(pLRT-Rig-FLAG) and A673(pLRT-Rig-FLAG) strains were established under reduced serum conditions. After 24 hours, one set of cultures was supplemented with 2 μg/ml doxycycline. At various time points over the course of 6 days, growth rates of the cultures were determined by taking total cell counts using a Coulter Counter.

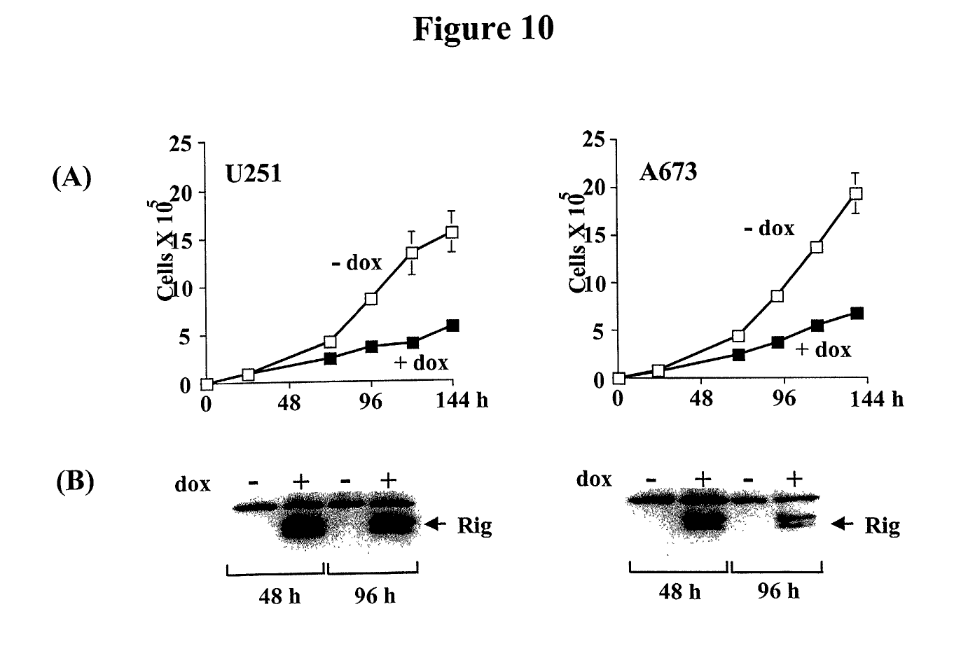

The resulting growth curves are shown in FIG. 10, Panel A, with the growth curves of the U251(pLRT-Rig-FLAG) cells on the left, and the growth curves of the A673(pLRT-Rig-FLAG) cells on the right. Cultures treated with doxycycline are shown with a solid square, while untreated cultures are shown with an open square. As can be seen in the Figure, both the U251 and A673 cell lines showed similar responses, where addition of the doxycycline resulted in the inhibition of cell growth compared to untreated cells. Results from these experiments indicate a direct correlation between increased Rig expression and reduced growth rate in neural tumor-derived cell lines.

In order to confirm doxycycline-induced Rig-FLAG expression, the doxycycline-treated and -untreated cells were collected, lysed and analyzed by Western blot using anti-Rig antiserum as the primary antibody (as described in Examples 9 and 11). The resulting blot is shown in FIG. 10, Panel B. As shown in this Figure, Rig-FLAG expression was strongly induced at 48 and 96 hours following drug treatment, but control, non-treated cells showed no Rig-FLAG expression at these same times. Identical results were obtained in another experiment using an anti-FLAG antibody as the primary antibody (data not shown). In both the U251 (pLRT-Rig-FLAG) and A673(pLRT-Rig-FLAG) cells, the induced Rig-FLAG expression was seen as early as 24 hours post-drug treatment and was sustained through 5 days post-treatment, whereas expression was not detectable in uninduced cells (data not shown). Therefore, Rig expression was rapidly upregulated by the addition of doxycycline, without apparent background expression.

Taken together, the above experiments indicate that Rig has activities that are antithetical to mitogenic Ras signaling. Specifically, these experiments indicate that Rig expression inhibits or stops cell growth, inhibits Ras-transformation, inhibits the growth of neural cell lines, and inhibits Ras signaling as measured by Elk-1 activity. However, knowledge of the mechanisms by which Rig antagonizes cell growth, Ras transformation, and Ras signaling is not necessary to make or use the present invention, nor is it intended that the present invention be limited to any particular mechanism(s). Nonetheless, each of these experimental results supports a conclusion that Rig has tumor-suppressor properties. Furthermore, as illustrated by the experiments described above, the present invention provides compositions and methods for the assessment of Rig activity. The present invention also provides numerous expression vectors suitable for the expression of Rig proteins.

IV. Rig Interacts with Raf-1

The ability of Rig and Raf-1 to interact in a transformed human embryonal kidney cell line (HEK-293T) was also examined. The ability of Raf to interact with H-Ras and K-Ras was also examined in the same assay. Raf-1 is a Ras-effector which binds to and is activated by Ras protein. These experiments employed a standard co-transfection/co-immunoprecipitation protocol, as known in the art. In this assay, cultured mammalian cells were cotransfected with FLAG-tagged and/or hemagglutinin (HA)-tagged mammalian expression vector constructs, followed by immunoprecipitation with an anti-Raf-1 antibody and Western immunoblotting using either anti-FLAG or anti-HA primary detection antibodies.

Briefly, a HA-RAF-1 fusion protein expression vector was produced by cloning the Raf-1 ORF (Fabian et al., Proc. Natl. Acad.Sci. USA 91:5982-5986 [1994]) into the pCGN mammalian expression vector, which also expresses the HA epitope (Fiordalisi et al, Methods Enzymol., 332:3-36 [2001]). A pCDNA3.1(FLAG) expression vector was produced by inserting the FLAG epitope in-frame upstream of the multiple cloning site of pCDNA3.1 (Invitrogen). A FLAG-Rig expression vector was made by cloning the Rig ORF into pCDNA-FLAG. HA-H-Ras and HA-K-Ras fusion genes were constructed by subcloning the H-Ras and K-Ras ORFs into pCGN.

HEK-293T cells were transfected with expression vector DNA using the calcium phosphate (CaPO₄) precipitation method. All cultures received the HA-Raf-1 expression vector. In addition, the cells were also co-transfected with either (a) the FLAG-Rig expression vector, (b) the HA-H-Ras expression vector, or (c) the HA-K-Ras expression vector.

Following transfection, the cells were cultured in reduced serum conditions for 48 hours. Cell lysates were then harvested in RIPA lysis buffer. HA-Raf-1 was immunoprecipitated from the lysates by incubation with a rabbit polyclonal anti-Raf-1 antibody. Immune complexes were immobilized and isolated with A/G Plus-agarose beads (Santa Cruz Biotechnology), resolved by SDS-PAGE, then blotted onto a polyvinylidene difluoride (PVDF) membrane. Western blots were probed with either a polyclonal anti-FLAG antibody (Sigma; M2 antibody) or an anti-HA antibody (BAbCo), followed by incubation with horseradish peroxidase-conjugated goat anti-rabbit IgG (Amersham). Antibody binding was then visualized, using techniques known in the art.

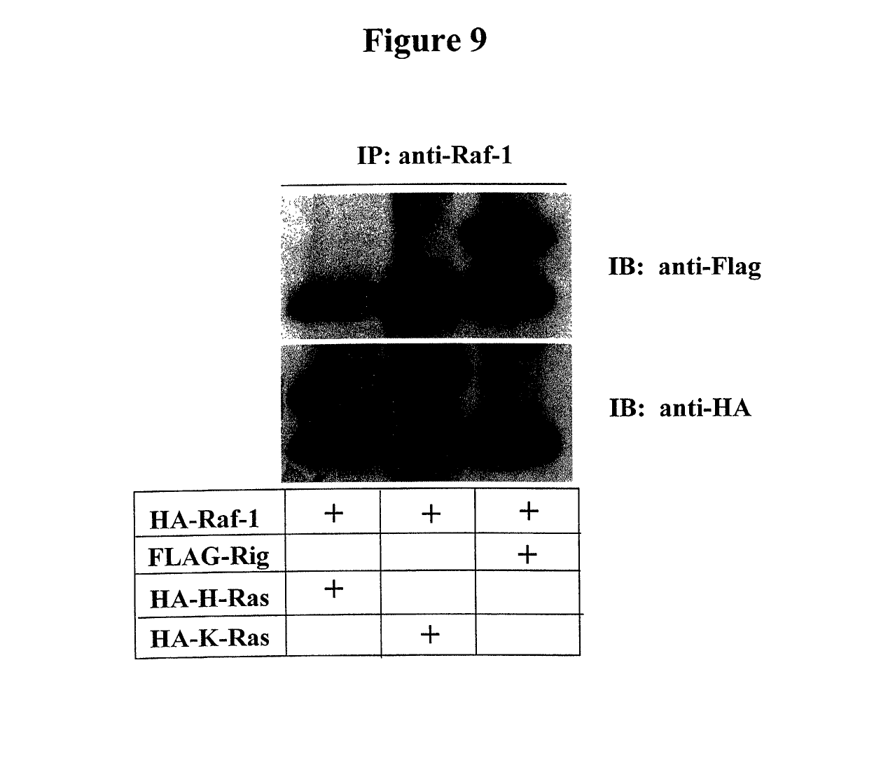

The Western blots showing the results of these binding assays are shown in FIG. 9. The top panel of this Figure shows a Western immunoblot using an anti-FLAG primary antibody, while the lower panel shows the same blot stripped and reprobed using an anti-HA primary antibody. The expression vectors used in each transfection are indicated at the bottom. As can be seen in the Figure, immunoprecipitation with the anti-Raf-1 antibody coprecipitated the recombinant HA-H-Ras, HA-K-Ras and FLAG-Rig proteins, indicating that each of these proteins are associated with Raf-1 in vivo. However, knowledge of the mechanism(s) by which Rig exerts its biological activities are not necessary to make or use the present invention, nor is it intended that the present invention be limited to any particular mechanism(s).

V. Generation of Anti-Rig Antibodies

The present invention provides polyclonal and monoclonal antibodies directed against the Rig protein. These antibodies find numerous uses, including diagnostic agents in the examination of tumor biopsy material, as well as in research. These clinical/diagnostic and research methods include but are not limited to immunoassays such as Western blotting, enzyme-linked immunosorbent assays (ELISAs), radioimmunoassays (RIAs), immunofluorescence assays (IFAs), immunoprecipitation, immunohistochemistry and immunoaffinity purification. All of these methods are well known in the art (See, e.g., Harlow and Lane (eds.), Antibodies: A Laboratory Manual, Cold Spring Harbor Laboratory Press [1988]; Ausubel et al. (eds.), Current Protocols in Molecular Biology, Vol. 1-4, John Wiley & Sons, Inc., New York [1994]; and Laurino et al., Ann. Clin. Lab Sci., 29(3):158-166 [1999]).

It is not intended that the present invention be limited to the antibody production methods provided below. Numerous methods for the production and purification of antibodies are well known in the art, and can be found in various sources (See e.g., Sambrook et al. (eds.), Molecular Cloning, Cold Spring Harbor Laboratory Press [1989]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). It is also not intended that the present invention be limited to any particular Rig antigen or epitope, nor any particular method for the production of Rig antigen. As skilled artisans know, numerous protocols for the purification of polypeptides suitable for use as antigens are available.

Production of Rig antigen: A variety of protocols and reagents are useful in the production of substantially purified Rig polypeptide for use as an antigen. In some embodiments of the present invention, the Rig antigen produced involves any portion of the Rig protein, where the portion is a minimum of 7 amino acids in length. In other embodiments, the Rig antigen is produced with or without a fusion protein tag (e.g., GST, HA or FLAG), while in still further embodiments, the Rig antigen is synthetic, recombinant or native. In additional embodiments, recombinant Rig antigen is produced in various cell types (e.g., bacterial cells or mammalian cells), while in still other embodiments, various expression vectors are used to drive expression of recombinant Rig protein within a cell. In further embodiments, the Rig antigen is purified by various methods (for example, including but not limited to, GST purification, as described herein). Indeed, it is not intended that the present invention be limited by the polypeptide purification protocols provided herein. It is contemplated that any protocol that will produce a substantially purified Rig polypeptide will find use with the present invention. Such alternative protocols include the use of hemagglutinin (HA)-tagged Rig fusion polypeptides, polyhistidine (i.e., 6xHis)-tagged Rig fusion polypeptides, thioredoxin-tagged Rig fusion polypeptides, and Rig polypeptides without any fused tag(s). In some embodiments, Rig polypeptides suitable for use as antigenic material are produced by synthetic chemistry.

Various protocols for recombinant polypeptide production also find use in the present invention. In some embodiments of the present invention, various host systems are used to produce starting material for Rig purification. Such systems include insect cells with a baculovirus overexpression system (e.g., Sƒ9 or Sƒ21 cell lines), mammalian cell lines used in conjunction with vectors designed for recombinant polypeptide overexpression (expression vectors, e.g., pZipNeo and pCDNAFLAG), or mammalian cells or tissues for the purification of Rig polypeptide expressed from its endogenous (i.e., native) chromosomal location. The cultivation of the transformed, transfected or infected host of the invention is carried out in a medium under conditions most appropriate for the growth of that particular host cell. These media formulations and culture conditions are well known to those in the art.

Polyclonal Anti-Rig Antisera Production: A polypeptide from the C-terminus of the Rig protein was used to produce anti-Rig polyclonal antiserum. Specifically, a 20 amino acid synthetic peptide corresponded to amino acids 175 to 194 of the human Rig protein, having the sequence:

L¹⁷⁵NIDGKRSGKQKRTDRVKGK (SEQ ID NO:8) was used to immunogenize two 3-9 month old New Zealand white rabbits using methods standard in the art. Immune serum was subsequently collected from these animals using standard techniques. This particular peptide was chosen based upon the uniqueness of its amino acid sequence as determined by database query. However, it is not intended that the present invention be limited to this specific amino acid sequence, as other sequences find use with the present invention.

For immunogen preparation, the Rig peptide was conjugated to keyhole limpet hemocyanin (KLH) and emulsified by mixing with an equal volume of Freund's Adjuvant. The amount of antigen utilized per immunization was 0.1 mg, injected into three subcutaneous dorsal sites. The animals received boosts at 2, 6 and 8 weeks. Auricular artery blood samples were obtained at weeks 4, 8, and 10. The serum was subsequently tested for the presence of anti-Rig antibody and stored at −20° C. until needed. The antiserum obtained from the week 10 bleed was used in the experiments described herein.

The antibody titer was determined with an enzyme linked immunosorbent assay (ELISA) with peptide (i.e., SEQ ID NO:8) bound in solid phase at a concentration of 1 μg peptide/well. Detection in the ELISA was accomplished using a biotinylated anti-rabbit IgG, horseradish peroxidase-strepavidin (HRP-SA) conjugate, and ABTS subtrate (Kirkegaard and Perry Laboratories), and absorbance was measured by OD₄₀₆ₙₘ. Only one of the two rabbits immunized produced Rig-reactive antibody. The anti-Rig antibody showed no cross-reactivity with any of the other monomeric GTPase GST fusion proteins tested, including Rap, Ras, Rheb and RRP22 (data not shown).

It is not intended that the present invention be limited to the polyclonal antisera production method described herein, as those in the art know numerous alternative reagents and protocols that find use with the present invention. For example, in some embodiments of the present invention, full-length Rig polypeptide, any portion thereof, either native, recombinant or synthetically produced, can be used to raise polyclonal antisera in any suitable animal (e.g., rabbit, rat, mouse, goat, etc.). In some embodiments, the antigen is mixed with an alternative adjuvant (e.g., Freund's incomplete or complete adjuvant) prior to immunization. In addition in some embodiments, the dosage of the antigen administered per animal ranges between 0.1 and 10 mg when no adjuvant is used, and between 1.0 and 100 μg when an adjuvant is used. In other embodiments, antigen is injected via intravenous, subcutaneous or intraperitoneal routes, and it is not intended that the interval of immunization, boosts or serum collection be limited to specific time points.

Monoclonal Antibody Production: For preparation of monoclonal antibodies directed toward the Rig protein, or any portion thereof, any technique that provides for the production of monoclonal antibody by continuous cell lines in culture can be used. These methods include but are not limited to the hybridoma technique originally developed by Köhler and Milstein (Köhler and Milstein, Nature 256:495-497 [1975]), as well as the trioma technique, the human B-cell hybridoma technique (See e.g., Kozbor et al. Immunol. Today 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

In some embodiments, the following protocol is used to produce a monoclonal antibody specific for a Rig protein of the present invention. It is not intended that the present invention be limited to the use of this or any other protocol, as numerous protocols for generating antibody-producing cells are known, and find use in the present invention.

Inoculation and Recovery of Antibody-Producing Cells: A mammalian animal host is immunized according to the protocol described above to produce polyclonal antisera. Subsequently, at 1 to 10 days, preferably 3 days, after the final immunization, antibody-producing cells are collected from the animal. Antibody-producing B cells found in the spleen, lymph nodes, peripheral blood, etc. are typically enumerated after isolation. In most embodiments, the spleen or local lymph node cells are used in the following steps.

Cell Fusion and Formation of Hybridoma Cell Lines: In order to obtain hybridomas which produce monoclonal antibodies, cell fusions between the antibody-producing B cells described above and myeloma cells are performed. Preferably, myeloma cells used for this purpose are unable to survive in HAT selective medium (i.e., containing hypoxanthine, aminopterin and thymidine), and are capable of surviving in this medium only when fused to B lymphocytes. In some embodiments, mouse myeloma cell strains including but not limited to, P3×63Ag.8.U1(P3U1), Sp2/0, NS-1 are used as myeloma cells. Subsequently, the myeloma cells and the antibody-producing B lymphocytes described above are subjected to cell fusion. In some embodiments, 1×10⁹ cells/ml of the antibody-producing cells and 1×10⁸ cells/ml of the myeloma cells are mixed together in equal volumes in cell culture medium (e.g., serum-free DMEM or RPMI-1640), and incubated in the presence of a cell fusion promoting agent. In some embodiments, polyethylene glycol with an average molecular weight of 1,500 Da is used as the cell fusion promoting agent. Alternatively, the antibody-producing cells and the myeloma cells are fused in a commercial cell fusion apparatus utilizing electric stimulation (e.g., electroporation).

Selection and Cloning of Hybridoma Lines: Following cell fusion, hybridomas are selected from the culture. In some embodiments, the cells are appropriately diluted in culture medium (e.g., RPMI-1640 medium containing fetal bovine serum), and plated in microtiter plate wells at a density of about 2×10⁵ cells/well. A selective medium is added to each well, and the fused cells are incubated in this selective medium. After unfused cells have died (usually by 14 days after the start of cultivation in the selective medium) hybridomas are ready to be screened.

Screening is performed in order to determine whether the desired antibody is present in the hybridoma culture supernatant. Any suitable method for screening of hybridomas finds use with the present invention. For example, in some embodiments, part of the culture supernatant of a well in which a hybridoma is grown is collected and subjected to enzyme immunoassay or radioimmunoassay.

Cloning of the fused cell is performed by the limiting dilution method or the like. Finally, the hybridoma of interest producing the monoclonal antibody of interest is established.

Production of Monoclonal Antibody Stocks: In some embodiments of the present invention, conventional cell culture methods or the abdominal dropsy formation method are employed for recovering the monoclonal antibody from the established hybridoma of interest (i.e., a monoclonal antibody-producing cell).

In the cell culture methods, the established hybridoma is cultured in a cell culture medium (e.g., RPMI-1640 or MEM medium, containing fetal bovine serum, or in a serum-free medium) under conventional culture conditions (e.g., at 37° C. in the presence of 5% CO₂) for 2 to 10 days. Then, the monoclonal antibody is then recovered from the culture supernatant.

In the abdominal dropsy or ascites fluid formation method, about 1×10⁷ cells of the hybridoma are administered into the abdominal cavity of an animal syngeneic to the animal from which the myeloma cells were derived, in order to propagate the hybridoma. One to two weeks thereafter, the abdominal dropsy/ascites fluid and/or serum is collected.

Antibody Purification: Following the production of polyclonal or monoclonal antibodies, the antibodies are purified using any suitable method known in the art, including but not limited to Protein A/Protein G affinity, ammonium sulfate salting out, ion exchange chromatography, gel filtration, affinity chromatography, or any of these methods in combination (See, e.g., Sambrook et al. (eds.), Molecular Cloning, Cold Spring Harbor Laboratory Press [1989]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). In view of numerous alternative protocols known in the art for the production and purification of polyclonal and monoclonal antibodies, it is not intended that the present invention be limited to any particular method.

VI. Analysis of Rig Protein Expression

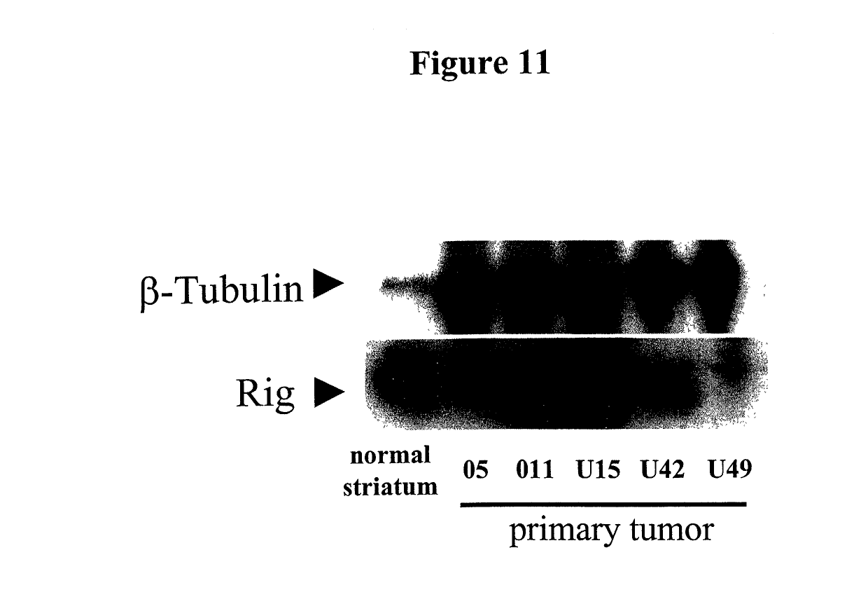

Endogenous human Rig protein expression was examined in normal tissue and neural tumorigenic samples using a Western immunoblotting technique. The anti-Rig polyclonal antiserum used in this method is described in Example 9. This example also describes experiments to test the ability of the polyclonal anti-human Rig antiserum to detect Rig protein in mouse tissue. The results of these experiments are shown in FIGS. 11, 12 and 13. Thus, the present invention provides means for the detection of Rig polypeptides.

In one experiment, protein lysates were prepared from human neural tissue (normal striatum), oligodendroglioma (05 and 011) and end-stage astrocytoma (U15, U42 and U49) and resolved by SDS-PAGE, and transferred to PVDF membrane (Immobilon-P membrane; Millipore). Western immunoblotting analysis was performed, using the anti-Rig antiserum as the primary antibody and goat anti-rabbit HRP conjugate as the secondary antibody, as described in Example 9. As per methods common in the art, prior to probing, blots were incubated for one hour in a blocking buffer, incubated with the primary antibody, washed, incubated with the secondary antibody, then washed again. Membranes were then bathed in enhanced chemiluminescent substrate (ECL; Amersham) and signal was detected by autoradiography. As a control, the same immunoblot was stripped and reprobed using an anti-β-tubulin primary antibody (Santa Cruz Biochemicals) and goat anti-rabbit secondary antibody (Amersham).

These two blots are shown in FIG. 11. As can be seen in the lower blot in the Figure, Rig expression is observed in the normal striatum tissue. However, Rig expression is greatly diminished or absent in 3 of 5 of the tumor tissues tested. Detection of the β-tubulin control protein confirmed that protein samples from the tumor specimens were not degraded or underloaded.

In another experiment using this same immunoblotting protocol, Rig expression was examined in a series human astrocytoma tumor tissue samples. The tumor samples used in this experiment were placed in two clinical groups, low-grade tumors (13 samples) and high-grade tumors (6 samples), where the low-grade tumors were determined to be less advanced, and the patients had a more favorable prognosis than those with high-grade tumors. Protein lysates were prepared from normal human neural tissue (normal striatum) and the astrocytoma tumor tissues, and equivalent amounts of total protein from the lysate samples were resolved by SDS-PAGE, transferred to PVDF membrane, and subject to the same Western immunoblotting analysis described previously, using the anti-Rig antiserum as the primary antibody, and goat anti-rabbit-HRP as the secondary antibody. Samples of normal human striatum tissue (labelled N) were also run in parallel.

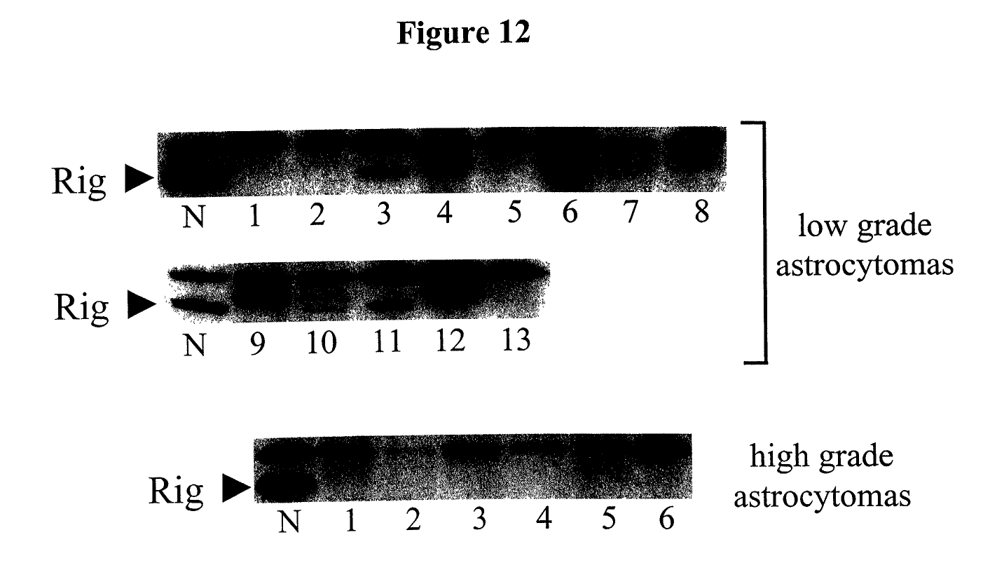

The resultant immunoblots are shown in FIG. 12. As indicated in this Figure, a protein species that cross-reacts with the anti-Rig antiserum was observed in the human normal striatum tissue. In the low grade astrocytoma tumor samples, the protein was absent or reduced in 8 of 13 tumor specimens. More strikingly, the anti-Rig polypeptide was found to be completely absent in 5 of 6 high grade astrocytoma specimens, and greatly reduced in the one remaining high grade specimen. Thus, based on the results of the experiments shown in FIGS. 11 and 12, Rig polypeptide expression is inversely correlated with tumorigenicity.

Another experiment was conducted using this same immunoblotting protocol to determine whether the anti-Rig antiserum produced in response to a human Rig peptide reacts with Rig protein from the mouse (see, Example 9). Protein lysates were prepared from human neural tissue (normal striatum) and mouse brain. Equivalent amounts of total protein from the human and mouse tissue lysate samples were resolved by SDS-PAGE, transferred to PVDF membrane, and subject to the same Western immunoblotting analysis described above.

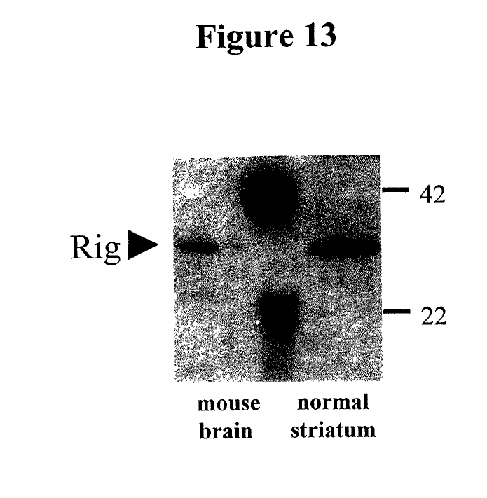

This immunoblot is shown in FIG. 13. Molecular weight markers are in the center lane. As can be seen in the Figure, a protein species of uniform size with an approximate molecular weight of 30 kDa, which cross reacts with the anti-Rig antiserum is present in both the human normal striatum and the mouse brain tissue. Thus, the antiserum raised against the human Rig peptide appears to detect a mouse Rig protein, as well as the human Rig protein. Also as indicated in FIG. 5, a human Rig nucleic acid probe detected a transcript in a total RNA sample from the mouse NE115 neuroblastoma cell line.

Thus, the present invention provides compositions and methods for the detection of Rig polypeptide from a wide variety of samples, including whole tissues as well as cultured cells.

VII. Analysis of Rig Prenylation

The prenylation of Rig, Ras and Rap was studied in an in vitro prenylation assay. Radiolabelled farnesyl pyrophosphate, radiolabelled geranylgeranyl pyrophosphate and radiolabelled mevalonate were alternatively used as substrates in the assay system for the determination of the prenylation specificity of each GTPase protein. In addition, the ability of a farnesyl transferase inhibitor (FTI; namely, FTI-277, Calbiochem, Catalog No. 344555) to inhibit the prenylation of Rig, Ras and Rap was examined. The prenylation experiments used glutathione-S-transferase (GST)-Rig, GST-H-Ras(G12V) and GST-Rap fusion proteins produced and purified in vitro. The H-Ras(G12V) protein was included in the prenylation assays as a positive control (Clark et al, Proc. Natl. Acad. Sci. USA 90:4887-4891 [1993]).

The GST-Rig and GST-H-Ras(G12V) fusion constructs were made by cloning the Rig ORF into the pGEX-2T plasmid (Pharmacia). The GST-Rap fusion construct was produced as described in Clark et al. (Clark et al., J. Biol. Chem., 272:10608-10615 [1997]). The resulting plasmids were transformed into competent bacteria, and the GST-fusion proteins produced and purified by standard techniques known in the art, and according to the manufacturer's protocol (Pharmacia), with the minor modification of the inclusion of 100 μM GTP and the omission of EDTA from the protein collection/purification buffers. Briefly, bacteria were transformed with the DNA vectors, and grown to exponential phase, at which time isopropyl-β-thiogalactopyranoside (IPTG) was added to the culture. Four hours following IPTG induction, the bacteria were pelleted, resuspended in lysis buffer, sonicated, then clarified by centrifugation. The resulting lysate was used as starting material for glutathione-conjugated agarose bead purification (Sigma) using standard techniques known in the art. Following their purification to near homogeneity, concentrations of the GST fusion proteins were estimated using SDS-PAGE followed by Coomassie Blue staining.

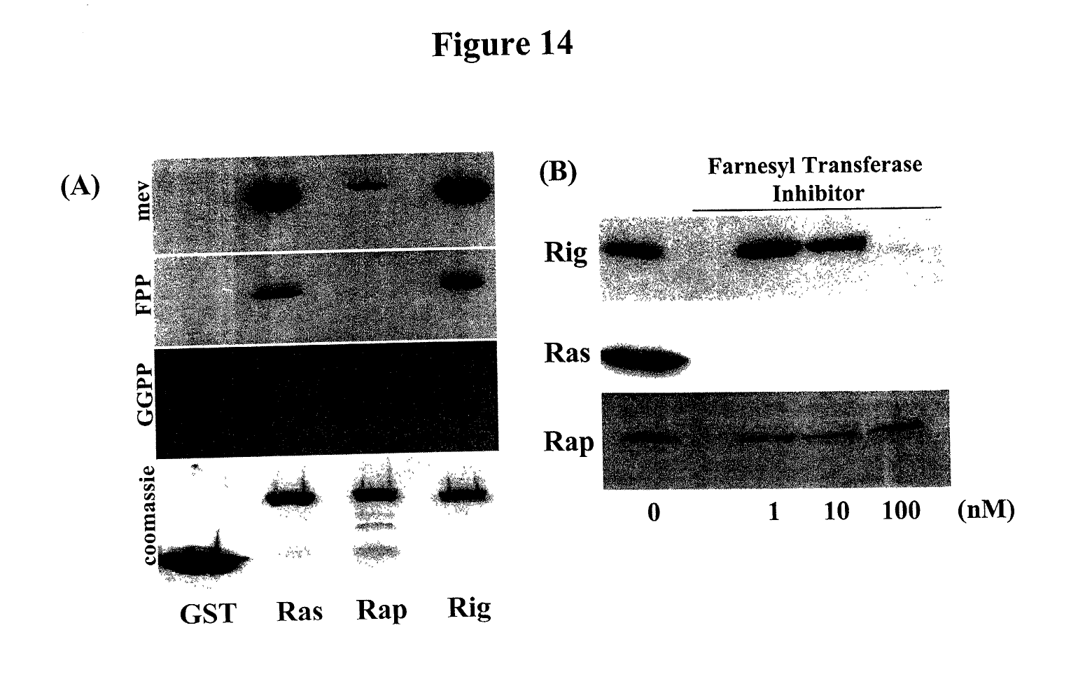

Each in vitro prenylation reaction contained, alternatively, GST, GST-Rig, GST-H-Ras(G12V) or GST-Rap. Each reaction mixture also contained rabbit reticulocyte lysate (Promega, Catalog No. L4151) with either 2.5 μCi [³H]-mevalonate (mev), 2.5 μCi [³H]-farnesyl pyrophosphate (FPP), or 2.5 μCi [³H]-geranylgeranyl pyrophosphate (GGPP). Reactions were incubated for one hour at 30° C. Affinity complexes were isolated by centrifugation, resolved by SDS-PAGE, fixed, soaked in autofluor (National Diagnostics, Catalog No. LS315), and analyzed by autoradiography. As shown in FIG. 14, Panel A, mevalonate (mev) was found to be a suitable substrate for the prenylation of GST-Rig, GST-Ras(G12V) and GST-Rap, while farnesyl pyrophosphate (FPP) was a suitable substrate for the prenylation of GST-Rig and GST-Ras(G12V), and geranylgeranyl pyrophosphate (GGPP) was a suitable substrate for the prenylation of Rig and Rap. Also as shown in FIG. 14, Panel A, GST protein was not prenylated by any of the three prenylation substrates. The bottom image of FIG. 14, Panel A, shows a Coomassie blue-stained polyacrylamide gel of the proteins used in the prenylation reactions, confirming that each protein was accurately synthesized, undegraded, and used in approximately the same concentration in each reaction. Thus, Rig undergoes post-translational prenylation, being modified by farnesyl and geranylgeranyl isoprenoid groups under these conditions.

In a second experiment, the sensitivity of mevalonate prenylation of GST-Rig, GST-Ras and GST-Rap to increasing amounts of farnesyl transferase inhibitor (FTI-277; Calbiochem) was assessed. The same methodology as previously described was used in this experiment. The in vitro prenylation assays contained, alternatively, equal amounts of GST-Rig, GST-Ras(G12V) or GST-Rap. Each reaction mixture contained rabbit reticulocyte lysate, 2.5 μCi [³H]-mevalonate and increasing concentrations of FTI-277 ranging from 1-100 nM. Reactions were incubated, then resolved by SDS-PAGE. The gel was fixed, soaked in autofluor and analyzed by autoradiography. As shown in FIG. 14, Panel B, prenylation (i.e., radiolabelling) of GST-Rig and GST-Ras was sensitive to FTI-277. Of the two proteins, GST-Ras showed a greater sensitivity, although farnesylation of both proteins was completely abolished at 100 nM FTI concentration. GST-Rap farnesylation by mevalonate did not show sensitivity to FTI in the 1-100 nM FTI concentration range. However, an understanding of the mechanisms regulating Rig prenylation, or any other mechanism regulating Rig function, is not required to make and use the present invention.

VIII. Analysis of Rig Subcellular Localization

The subcellular localization of the Rig protein was also analyzed. To accomplish this, a green fluorescent protein (GFP) fusion was constructed to yield a GFP-Rig chimeric indicator protein. Following transient expression of this protein in a host cell, the fusion protein was visualized and photographed. An ecdysone-inducible, two part mammalian conditional expression system was used in these experiments to conditionally express the Rig-GFP fusion protein, according to the manufacturer's protocols (Invitrogen).

A vector comprising the Rig and GFP coding sequences, operably joined in-frame, was engineered using the two-component pIND(SP1) inducible expression vector system (Invitrogen). In this system, the ORF of interest is placed under the transcriptional control of the ecdysone promoter, and the ecdysone receptor is constitutively expressed from a second plasmid. In this experiment, the Rig ORF as well as GFP, were cloned in operable combination, into pIND(SP1), yielding GFP-Rig/pIND (SP1). Expression of the GFP-Rig coding sequence in a mammalian cell stably cotransfected with the pVgRXR plasmid (which constitutively expresses the heterodimeric ecdysone receptor; Invitrogen) is inducible by the addition of the ecdysone-analog molecule ponasterone A.

The ecdysone-inducible vector encoding the GFP-Rig fusion protein was transiently transfected into a human embryonal kidney (HEK)-293-based cell line (E293), which constitutively expresses the ecdysone receptor subunits from the pVgRXR plasmid (Invitrogen). The transfected cells were exposed to ponasterone A 48 hours following transfection and were viewed 24 hours post-ponasterone A induction. GFP fluorescence was visualized using an inverted fluorescence microscope with a 40× objective under excitation/emission wavelengths of 488 nm and 507 nm, respectively. A parallel control experiment was conducted using an ecdysone-inducible expression vector containing the GFP open reading frame fused to a nuclear localization signal (NLS).

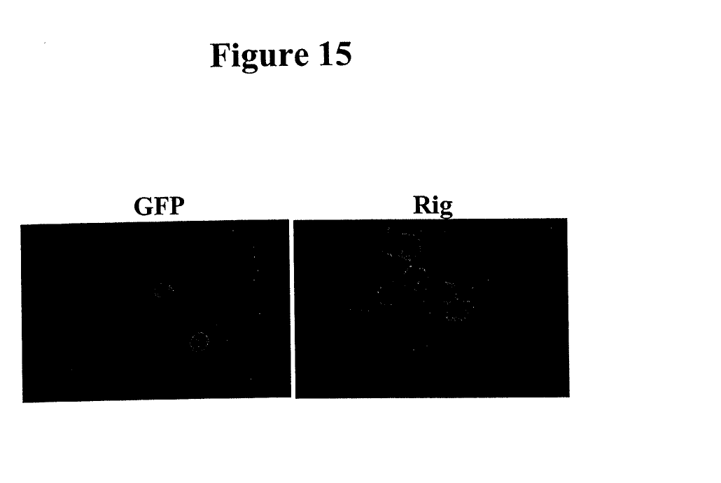

Fluorescence microscopy images are shown in FIG. 15. Cells that express GFP-NLS are shown in the left panel, while the right panel shows an image of cells expressing the GFP-Rig fusion protein. Largely nuclear fluorescence was observed in cells expressing the GFP-NLS fusion, while plasma membrane localization is clearly present in cells expressing the GFP-Rig chimera. Thus, like Ras proteins, Rig also appears to localize to the plasma membrane. However, an understanding of the mechanism(s) regulating Rig subcellular localization, or any other mechanism(s) regulating Rig function, is not required to make and use the present invention. Nor is it intended that the present invention be limited to any particular mechanism(s).

IX. Compositions and Methods for the Detection of the Rig Gene, Transcript and Protein

As indicated above, the present invention provides the Rig gene, which has tumor-suppressor properties. Also as described herein Rig was found to be expressed in all normal tissues tested, and loss of Rig expression was observed in a majority of tumor tissues and transformed cell lines tested.

It is contemplated that assessment of Rig expression will find use in various settings, including but not limited to basic research, as well as for cancer diagnostics. In one embodiment, the assessment of Rig expression will find use in used in making cancer treatment decisions (i.e. whether to treat a subject with Rig gene therapy). Prior to treatment, it is contemplated that a biopsy sample taken from a subject's tumor is analyzed for Rig expression and/or genomic Rig status. Furthermore, it is contemplated that subjects whose tumors display chromosomal loss or mutation of the Rig gene are the most likely to benefit from gene therapy with a recombinant Rig gene.

In addition, the present invention provides compositions and methods for the assessment of endogenous Rig expression. In some embodiments, these methods and compositions are used alone or in combination, and include Northern blotting to detect endogenous Rig RNA; PCR analysis of cellular RNA to detect Rig transcripts; Western immunoblotting using an anti-Rig antibody to detect Rig polypeptide; and ELISA to detect or quantitate Rig polypeptide. Thus, the present invention provides means to assess Rig protein presence and function in various samples. Some of these methods are discussed in greater detail below.

1) Northern Blotting to Detect Endogenous Rig RNA

The present invention provides Northern blotting methods for the detection of endogenous Rig transcripts, as described in Examples 3 and 4. As described in these Examples, total cellular RNA was isolated using a commercial RNA isolation kit (i.e., Qiagen RNeasy Total RNA Mini Kit). The RNA was resolved using denaturing agarose electrophoresis, blotted, and probed using a random-primed ³²P-dCTP labelled probe corresponding to the full-length Rig cDNA.

In view of numerous alternative protocols known in the art for Northern blotting, it is not intended that the present invention be limited to the Northern blotting protocol provided in Examples 3 or 4, nor any other particular Northern blotting method. For example, in some embodiments, RNA is isolated from tissue samples using other methods (e.g., guanidinium isothiocyanate lysis followed by cesium chloride gradient purification). Alternatively, polyA+mRNA can be used with the Northern blotting methods of the present invention.

Similarly, alternative probe synthesis and labelling techniques also find use with the present invention. For example, any probe having a minimum complementarity of 25 bases to the Rig RNA will find use in the Northern blot methods of the present invention. Furthermore, it is contemplated that the nucleic acid comprising the probe will be generated by PCR, by restriction digest, or by synthetic oligonucleotide synthesis. Other methods for labelling nucleic acid probes also find use with the present invention (e.g., labelling with ³³P radioisotope or non-radioactive labelling methods). In addition, various Northern blotting protocols and reagents suitable for use in the present invention are known in the art (See e.g., Ausubel et al. (eds.), Current Protocols in Molecular Biology, Vol. 1, pages 4.9.1-4.9.16, John Wiley & Sons, Inc., New York [1994]).

2) PCR Analysis of Cellular RNA to Detect Rig Transcripts

In some embodiments, an mRNA transcript of the Rig gene is detected in total cellular RNA or polyA⁺ mRNA using reverse transcription polymerase chain reaction (RT-PCR). This technique, which incorporates both reverse transcriptase activity (i.e., an RNA-dependent DNA polymerase) and DNA-dependent DNA polymerase activity, is described in multiple references (e.g., Mullis et al. (eds.), PCR—The Polymerase Chain Reaction, Chapter 24, “RT-PCR and Gene Expression,” Birkhauser Publishers, Cambridge, Mass. [1994]; and Ausubel et al. (eds.), Current Protocols in Molecular Biology, Section 15.4, “Enzymatic Amplification of RNA by PCR,” John Wiley & Sons, Inc., New York [1994]). In a preferred embodiment, the reverse transcriptase and DNA-dependent DNA polymerase activities are encoded by the same enzyme. In a most preferred embodiment, the enzyme having both reverse transcriptase and DNA-dependent DNA polymerase activities is thermostable. It is also not intended that the present invention be limited to any particular method of RNA isolation. The art knows well alternative protocols for the isolation of total RNA or polyA⁺ mRNA.

3) Western Immunoblotting Using an Anti-Rig Antibody to Detect Rig polypeptide

The present invention provides monoclonal and polyclonal antibodies directed against the Rig polypeptide. The anti-Rig antibodies of the present invention find use in Western immunoblotting to detect recombinant or endogenous Rig polypeptide. For example, these antibodies find use in assessment of endogenous Rig polypeptide expression in tumor biopsy samples taken from human subjects.

In view of the numerous conditions known in the art for the analysis of proteins by Western immunoblotting, it is not intended that the present invention be limited to any particular Western blotting method. For example, in some embodiments, tissue biopsy samples to be analyzed by Western blotting using the anti-Rig antibody of the present invention, are prepared by homogenization either manually (e.g., using a Dounce homogenizer) or mechanically (i.e., electric homogenizer). Before, during or after homogenization, tissue samples are suspended in a sample buffer suitable for loading directly onto an SDS-PAGE gel (e.g., Laemmli buffer). Following homogenization and addition of a suitable sample buffer, samples are heated, typically at 95° C. for 2 minutes, loaded and resolved by SDS-PAGE, blotted onto a suitable membrane (e.g., polyvinylidene difluoride [PVDF]), probed with an anti-Rig antibody of the present invention, and visualized with an appropriate secondary antibody. Protocols and reagents for Western immunoblotting are well known to those in the art (See e.g., Ausubel et al. (eds.) (Current Protocols in Molecular Biology, Section 10.8, “Immunoblotting and Immunodetection,” John Wiley & Sons, Inc., New York [1994]; and Walker (ed.), The Protein Protocols Handbook, Part III, “Blotting and Detection Methods,” Humana Press, Totowa, N.J. [1996]).

4) ELISA Assay to Detect or Quantitate Rig Polypeptide

As indicated above, the present invention provides monoclonal and polyclonal antibodies raised against a Rig polypeptide. The anti-Rig antibodies of the present invention find use in enzyme-linked immunosorbent assays (ELISAs) to detect and/or quantitate recombinant or endogenous Rig polypeptides (e.g. endogenous Rig polypeptide in a tumor biopsy sample taken from a human subject).

Numerous ELISA methods are known in the art (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). Some ELISA formats known in the art which find use with the present invention include “direct ELISAs,” “indirect ELISAs,” and “sandwich ELISAs.” Thus, in view of the numerous ELISA formats known in the art, it is not intended that the present invention be limited to any particular ELISA format.

Briefly, in some embodiments, these ELISA methods first immobilize a protein of interest that is in a sample (e.g., a protein extract from a tumor tissue biopsy, in which the tumor is suspected of having an altered Rig polypeptide expression profile) to a solid support (e.g., a microtiter plate well). In some embodiments, this immobilization is directly to the solid support, while in other embodiments immobilization occurs via a suitable “capture antibody.” The anti-Rig antibody of the present invention finds use as a Rig-specific capture antibody. Detection and quantitation of the immobilized antigen (i.e., the Rig polypeptide) is accomplished by the use of an antibody-enzyme conjugate detection antibody (i.e., the anti-Rig antibody of the present invention conjugated to a suitable enzyme) capable of binding to the immobilized antigen and producing a quantifiable signal. The amount of enzyme reaction product produced after the addition of a suitable enzyme substrate is directly proportional to the amount of antigen present in the sample. Enzymes commonly used in the ELISA detection step include horseradish peroxidase (HRPO), urease, alkaline phosphatase, glucoamylase and β-galactosidase. Methods for the preparation of suitable antibody-enzyme conjugates are also known to those skilled in the art. The end product of an ELISA is a signal, typically the development of color or fluorescence. Color development and fluorescence are read (i.e., quantitated) using a suitable spectrocolorimeter (i.e., a spectrophotometer) or spectrofluorometer, respectively. The amount of color or fluorescence is directly proportional to the amount of immobilized antigen.

The present invention further provides diagnostic kits useful for the rapid assessment of Rig mRNA or polypeptide expression using either Northern blotting, PCR analysis, Western blotting, and/or an enzyme-linked immunosorbent assay (ELISA).

In some embodiments, kits designed to incorporate reagents for use in PCR methods include, but are not limited to, nucleic acid isolation reagents, PCR primers, PCR reaction buffer, deoxyribonucleotide triphosphates (dNTPs), a thermostable reverse transcriptase and a thermostable DNA-dependent DNA polymerase (the latter two components can be substituted with a thermostable enzyme having both reverse transcriptase and DNA-dependent DNA polymerase activities). In alternative embodiments, kits designed to facilitate Northern blotting include, but are not limited to, RNA purification reagents, sample denaturation buffer, suitable blotting membrane (e.g., PVDF), a nucleic acid probe, and hybridization and wash buffers. In still further embodiments, kits designed to facilitate Western blotting include, but are not limited to, tissue homogenizers, protein extraction buffers, SDS-PAGE sample buffers, suitable blotting membrane, blocking and wash buffers, suitable primary and secondary antibodies and visualization reagents. In related immunoassay based embodiments, kits designed to facilitate ELISAs include, but are not limited to microtiter plates, a suitable capture antibody, a suitable detection antibody (i.e., antibody-enzyme conjugate), suitable blocking and wash buffers, and substrate and stop solutions. In other embodiments, these kits further include any material(s) which make possible or facilitate the analysis of a sample, including, but not limited to, apparatus for sample collection, sample tubes, holders, trays, racks, dishes, plates, instructions to the kit user, samples to be used for standardization,/normalization, and/or positive and negative control samples.

X. Methods for Inhibiting Cell Growth

The present invention provides compositions related to the Rig gene. The experiments described herein demonstrate that the Rig gene has properties of a tumor suppressor gene. Furthermore, the present invention provides methods (See e.g., Examples 6, 8 and 10) for the inhibition of cell growth. Each of these methods involves use of a recombinant expression vector comprising the Rig ORF, and expression of that ORF to yield a Rig polypeptide product within a cell. It is not intended that a method to inhibit cell growth comprising the Rig gene be limited to the type of recombinant expression vector used, the means of delivery of the recombinant expression vector, or the recipient cell type to receive the recombinant expression vector.

The present invention provides various expression vectors for the expression of the Rig gene (e.g., a pCDNA3.1 based vector under the transcriptional control of the constitutively active CMV promoter, or the pLRT retroviral expression vector under a conditional tetracycline-responsive promoter). However, it is not intended that the present invention be limited to these or any other expression vectors, as those in the art know numerous other expression vectors suitable for use with the present invention. Such vectors may be under constitutive or conditional transcriptional control, and can be viral or non-viral based.

Similarly, it is not intended that the present invention be limited to any particular method for delivering a recombinant expression vector into a cell. Delivery of a DNA vector into a cell can be accomplished by a variety of means known to those in the art, including calcium phosphate (CaPO₄) precipitation, DEAE-dextran-mediated transfection, electroporation, microinjection, lipofection, retroviral infection, microinjection including biolistics and protoplast fusion. Where viral-based vectors are used, numerous viral sequences find use with the present invention, including but not limited to adenovirus sequences, adeno-associated virus sequences, retrovirus sequences, herpes virus sequences, vaccinia virus sequences and Moloney virus sequences. Mammalian cell transfection and viral infection techniques are common in the art, and are described in many sources (See, e.g., Ausubel et al. (eds.), Current Protocols in Molecular Biology, Vol. 1-4, John Wiley & Sons, Inc., New York [1994]).

Furthermore, it is not intended that the present invention be particularly limited to the type of cell to receive the recombinant Rig expression vector, as many types of eukaryotic cells receiving a suitable recombinant Rig expression vector are encompassed by the present invention. Moreover, the cell which receives the recombinant Rig expression vector can be cultured (in vitro), or within a subject (in vivo). For example, astrocytoma, glioblastoma, Ewing sarcoma, primitive neuroectodermal tumor, rhabdomyosarcoma, undifferentiated carcinoma, and neuroblastoma cells all find use with the present invention.

XI. Pharmaceutical Compositions Comprising the Rig Gene for the Treatment of Cancer

In particularly preferred embodiments, the present invention provides a polypeptide that has tumor suppressor activity (i.e., the Rig polypeptide), and a gene encoding the polypeptide. It is contemplated that these compositions will find use as therapeutic agents for the treatment of cancer, in view of this ability of the recombinant Rig gene of the present invention to inhibit or stop the growth of tumor cells.

When compositions of the present invention are used as therapeutic agents in the treatment of cancer, it is not intended that the present invention be limited to any particular type of cancer. For example, in one embodiment the present invention finds use in the treatment of cancers of neural origin, including but not limited to, astrocytoma cells, glioblastoma cells, Ewing sarcoma cells, primitive neuroectodermal tumor cells, rhabdomyosarcoma cells, undifferentiated carcinoma cells, and neuroblastoma cells. However, it is contemplated that the present invention will find use in the treatment of other cancers not catalogued herein.

In one embodiment, the present invention is used to slow, stop or eradicate cancer. In one embodiment, compositions of the present invention are delivered via a viral delivery system. In an alternative embodiment, a non-viral delivery system is utilized.

Viral-mediated gene delivery has been shown to be an effective mechanism for gene delivery. Indeed, methods for viral-mediated gene therapy have recently been shown to be effective in human and non-human systems (Cavazzana-Calvo et al., Science 288:669-672 [2000]; Kay et al., Nature Genetics 24:257-261 [2000]; Amado and Chen, Science 285:674-676 [1999]; Burton et al., Proc. Natl. Acad. Sci. USA 96(22):12725-12730 [1999]; Zhang, Cancer Gene Ther., 6(2):113-138 [1999]; Connelly et al, Blood 91(9):3273-3281 [1998]; and Connelly et al., Blood 88(10):3846-3853 [1996]). A number of viruses have proven to be effective tools in recombinant gene delivery protocols. These viral vectors include but are not limited to, adenovirus (lentivirus) vectors, adeno-associated virus vectors, herpes virus vectors, vaccinia virus vectors, and retrovirus vectors. In some preferred embodiments, the recombinant viral vector comprising the Rig gene of the present invention comprises nucleic acid elements operably linked for the purpose of transcribing and translating the gene of the invention in tumor cells in a subject. In other preferred embodiments, these nucleic acid elements consist of a nucleotide sequence encoding the Rig polypeptide, and operably linked promoter and enhancer elements for expression of the Rig gene. In some embodiments, these promoter/enhancer elements are widely active in all or many cell types, and direct constitutive expression of the gene (e.g., cytomegalovirus (CMV), SV40 or Rous sarcoma virus (RSV) promoter/enhancer sequences). In alternative embodiments, operably linked promoter/enhancer elements are restricted in activity to a single cell type or tissue (e.g., cardiac-specific, liver-specific or ovarian-specific promoter/enhancers) (Maniatis et al., Science 236:1237-1245 [1987]; Voss et al., Trends Biochem. Sci., 11:287 [1986]). In other embodiments, a promoter/enhancer element that imparts inducible (i.e., conditional) expression of an operably linked open reading frame (e.g., tetracycline inducible or repressible promoters) is used. Furthermore, operably linked nucleotide sequences such as sequences directing proper translation initiation, post-transcriptional splicing/editing, and/or polyadenylation are included in some embodiments. In still other embodiments, in addition to containing nucleotide sequences controlling the expression of the Rig gene, a viral gene therapy vector further contains the necessary nucleotide sequences for in vitro replication and propagation of the virus, production of infective virion particles, and sequences that impart stability to the DNA in a cellular host (although many viral functions require the presence of a “helper virus”). Collectively, such sequences are sometimes referred to as the viral “backbone.”

In alternative embodiments, non-viral delivery systems are used to deliver the Rig gene as a gene therapy agent. Non-viral delivery systems include gene delivery by direct application of the nucleic acid to cells or tissues, or the use of phospholipid vesicles such as liposomes (Mahato et al., Adv. Genet., 41:95-156 [1999]). Liposomes are biodegradable vesicles containing an internal aqueous region surrounded by a lipid bilayer. This structure is able to encapsulate materials (e.g., at least one gene of the present invention). By mixing at least one gene of the present invention with phospholipid starting material under appropriate conditions, a liposome-gene complex forms. Subsequently, when this complex is cultured with cells or administered to cells in a subject, the gene(s) in the complex is taken into the cells (i.e., via lipofection). Thus, in some embodiments, nucleic acid of the present invention is enclosed in phospholipid vesicles or liposomes, and the resultant liposomes are administered to a subject, or to the tumor of the subject.

In still other embodiments, beads (e.g., DYNAFECT beads) coated with antibodies specific for defined cell surface antigens are used to deliver or enhance the transmembrane uptake of nucleic acid (Bildirici et al., Nature 405:298 [2000]). This process, also known as immunoporation, delivers DNA to cells at a high rate of efficiency, and offers the added benefit of targeting the particular cells in a mixed population of cells, to receive the gene of interest (i.e., the Rig gene). In further embodiments, this technology is used to directly deliver the Rig protein of the present invention to the site of a tumor or other target cells.

In some embodiments, methods for the delivery the Rig gene to a subject involve parenteral administration. In some embodiments, systemic administration of the Rig gene is by intravenous or intra-arterial administration. In alternative embodiments, local administration is used. In one embodiment, local administration of the Rig gene is accomplished by surgical delivery, implantation, or injection, or any other suitable method that restricts the distribution of the gene. In still further embodiments, an administration method is combined with catheter techniques and surgical operations.

As known to those in the art, the dosage levels of the agent for delivering the gene(s) of the invention vary depending on the age, sex and conditions of the subject, the route of administration, the number of administrations, and the type of the formulation, among other considerations. One skilled in the art is capable of determining the therapeutically effective amount appropriate for any given situation. Usually, it is appropriate to administer a gene of the invention in an amount of 0.1-100 mg/adult body/day, although other concentrations are contemplated.

### Experimental

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); μg (micrograms); ng (nanograms); 1 or L (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); bp (base pair); PCR (polymerase chain reaction); RT (reverse transcription) kDa (kilodalton); ELISA (enzyme-linked immunosorbent assay); BSA (bovine serum albumin); FBS (fetal bovine serum); and Dulbecco's Modified Eagles Medium (DMEM).

Where manufacturers are indicated, the following abbreviations apply: Ambion (Ambion, Inc., Austin, Tex.); Amersham or Amersham/Pharmacia (Amersham-Pharmacia, Inc., Piscataway, N.J.); ATCC (American Type Culture Collection, Manassas, Va.); BAbCo (Berkeley Antibody Company/Covance Research Products/CRP, Inc.); Boehringer Mannheim (Boehringer Mannheim, Corp., Indianapolis, Ind.); Calbiochem (Calbiochem/Novabiochem Corp., La Jolla, Calif.); Clontech (Clontech, Palo Alto, Calif.); GCG (Genetics Computer Group, Madison, Wis., >http://www.GCG.com<); Gibco/BRL/Life Technologies (GIBCO BRL Life Technologies, Gaithersburg, Md.); Invitrogen (Invitrogen Corporation, Carlsbad, Calif.); KPL (Kirkegaard and Perry Laboratories, Gaithersburg, Md.); Millipore (Millipore Corporation, Bedford, Mass.); National Diagnostics (National Diagnostics, Atlanta, Ga.); NEB (New England Biolabs, Beverly, Mass.); Pharmacia (Amersham/Pharmacia Biotech, Piscataway, N.J.); PharMingen (PharMingen/Transduction Laboratories, San Diego, Calif.); Promega (Promega Corp., Madison, Wis.); Santa Cruz Biotechnology (Santa Cruz Biotechnology, Inc., Santa Cruz, Calif.); Sigma (Sigma Chemical Co., St. Louis, Mo.); and Stratagene (Stratagene Inc., La Jolla, Calif.).

Restriction enzymes, other DNA modification enzymes and molecular biology reagents used in these Examples are readily available from numerous manufacturers, including, but not limited to several of those listed above (Invitrogen, NEB, Boehringer Mannheim, Promega, Gibco/BRL and Stratagene).

The following Examples are provided in order to demonstrate and further illustrate certain preferred embodiments and aspects of the present invention and are not to be construed as limiting the scope thereof.

### EXAMPLE 1

### Cell Culture and Transfections

NIH-3T3 cells (a mouse, contact-inhibited embryonic cell line; ATCC CRL No. 1658) were propagated in Dulbecco's Modified Eagles Medium (DMEM; Gibco) supplemented with 10% fetal bovine serum (FBS; Gibco). COS-7 cells (SV-40 transformed, African Green Monkey kidney cell line; ATCC CRL No. 1651), HEK-293T cells (a transformed human embryonal kidney cell line, ATCC CRL No. 1573) and the Phoenix ecotropic retroviral producer cell line were propagated DMEM supplemented with 10% FBS. The PA317 amphotropic packaging cell line was grown in DMEM supplemented with 10% FBS. To produce cells capable of inducible-expression of a Rig-GPF chimeric protein, E293 cells were constructed by stably transfecting HEK-293T cells with the pVgRXR (i.e., the ecdysone receptor vector; Invitrogen). The resulting cell line was maintained in DMEM supplemented with 10% fetal bovine serum and penicillin/streptomycin, and kept under selective pressure using 5 μM Zeocin™ Invitrogen). Tumor-derived neural cell lines U251 (a human glioblastoma), TC32 (a human Ewing sarcoma), TC106 (a human PNET—primitive neuroectodermal tumor), A673 (human rhabdomyosarcoma/undifferentiated carcinoma; ATCC CRL No. 7910), SH5Y (a human neuroblastoma) and NE1 15 (a murine neuroblastoma) were cultured in RPMI-1640 (Gibco) supplemented with 10% FBS.

Cells were transfected using the calcium phosphate (CaPO₄) precipitation technique as known in the art (Clark et al., Methods Enzymol., 255:395-412 [1995]). Mammalian cells were cultured using conditions and techniques common in the art (See e.g., Ausubel et al. (eds.), Current Protocols in Molecular Biology, Vol. 4, Section A.3F, “Techniques for Mammalian Cell Tissue Culture,” John Wiley & Sons, Inc., New York [1994]). Reagents for the culture of cells are routinely available from a number of manufacturers, including but not limited to, Sigma and Gibco/BRL.

### EXAMPLE 2

### Electronic Screening and Cloning

In order to identify novel Ras-family gene products which have tumor suppressor activity, an electronic screen was undertaken to identify proteins showing homology to the Noey2 (also called Ahri1) gene product (Yu et al., Proc. Natl. Acad. Sci. USA 96:214-219 [1999]; GenBank Accession No. NP_{—}004666; and SEQ ID NO:9). The full length Noey2 amino acid sequence (SEQ ID NO:9) was used as the query sequence to search the databases of the National Center for Biotechnology Information (NCBI), using the search program “Advanced tBLASTn” (>http://www.ncbi.nlm.nih.gov/BLAST/<). This electronic screen identified an open reading frame (ORF) on a bacterial artificial chromosome (BAC) 41195 (GenBank Accession No. AC006538), corresponding to a region on human chromosome 19 at p13.3. This ORF was then used to search the NCBI expressed sequence tagged (EST) databases. That search identified an IMAGE Consortium EST (IMAGE Consortium Clone ID No. 2165313; GenBank Accession No. AI497811) containing a human ORF corresponding to the region of interest on the BAC 41195 clone. This IMAGE Consortium Clone was obtained from ATCC (Catalog No. 3363561) and used as template material in a PCR strategy to isolate the full length ORF from the cDNA clone using the following primers:

| | |
|---|---|
| 5′ primer: | |
| 5′-GGGGGATCCATGCCGGAACAGAGTAACGATTAC- | (SEQ ID NO:2) |
| 3′, | |
| 3′ primer: | |
| 5′-GCGGAATTCTCACATGAGGGTGCATTTGCCC-3′. | (SEQ ID NO:3) |

These primers contained terminal BamHI and EcoRI restriction sites, respectively, to facilitate subcloning. The resulting PCR product was subcloned into a variety of vectors for subsequent study, as detailed below.

This PCR strategy identified a 597 base pair human ORF, including the 3′ stop codon; shown in FIG. 1, Panel A; and SEQ ID NO:4). This ORF (i.e., gene and polypeptide) are herein called “Rig.”

The Rig ORF was predicted to encode a 198 amino acid polypeptide (shown in FIG. 1, Panel B; GenBank Accession No. AAD13119; and SEQ ID NO:5). The polypeptide encoded by the Rig ORF was compared to other Ras-family members using PileUp and GrowTree programs of the Genetics Computer Group (GCG) suite of sequence analysis tools. Sequence alignment and dendogram comparison of the Rig polypeptide with other Ras-family proteins is shown in FIGS. 2 and 3, respectively. The human Ras-related sequences of these comparisons include: Rig (GenBank Accession No. AAD13119; and SEQ ID NO:5), Noey2 (GenBank Accession No. NP_{—}004666; and SEQ ID NO:9), Ra1A (GenBank Accession No. TVHUAA; and SEQ ID NO:10), Rap1A (GenBank Accession No. P10113; and SEQ ID NO:11), Rap2A (GenBank Accession No. XP_{—}007223; and SEQ ID NO:12), HRas (GenBank Accession No. P01112; and SEQ ID NO:13), RRas (GenBank Accession No. P10301; and SEQ ID NO:14), and Rheb (GenBank Accession No. Q15382; and SEQ ID NO:15).

From these comparisons, it was determined that the Rig protein shares significant homology with a large number of Ras-family GTP-binding proteins, including Rnd3, Rap proteins, Ras proteins, Rheb and RRP22. Rig shares strongest homology to the Noey2 gene product (SEQ ID NO:9; and GenBank Accession No. NP_{—}004666), where the two proteins share 63% sequence similarity over their full length, 71% identity in the guanine nucleotide-binding domain and 77% identity in the effector domains. The Rig protein contains a C-terminal CAAX (SEQ ID NO:1) motif located at amino acid positions 195-198, which is a signal for post-translational prenylation. Surprisingly, the Rig protein contains a serine at amino acid residue 65, suggesting that the Rig protein does not have GTPase activity. In FIG. 2, the Rig protein guanine nucleotide binding domain is indicated in bold capital letters, the effector binding domain is indicated by underlined capital letters, and the CAAX (SEQ ID NO: 1) motif is simply capitalized.

### EXAMPLE 3

### Rig RNA Expression Analysis in Human Tissues

In these experiments, the expression pattern of the Rig gene was analyzed by Northern blotting in RNA samples isolated from a variety of human tissues, using both electrophoretic and slot-blot formats. A probe corresponding to the 597 base pair Rig ORF was used in these Northern blots.

In one experiment Rig mRNA expression was analyzed using a multiple human tissue poly(A)⁺ mRNA Northern blot (Clontech) probed with a Rig cDNA probe produced by random-primed ³²P-dCTP labelling (PRIME-IT® II random primer labeling kit; Stratagene) of the 597 bp Rig ORF. Hybridization and washing conditions were as per the manufacturer's recommended protocol (Clontech). Briefly, pre-hybridization and hybridization was performed in ExpressHyb™ hybridization solution (Clontech; Catalog No. 8015). The blot was then washed once with 2×SSC (where 20×SSC contains 3M NaCl and 0.3M Na₃citrate, pH 7.0) and 0.1% SDS for 15-30 min at 65° C., then washed with 0.1×SSC and 0.1% SDS for 10-20 min at 65° C., followed by autoradiography.

As shown in FIG. 4, Panel A, a single predominant transcript corresponding to the Rig gene was present in the poly(A)⁺ mRNA derived from brain and heart. No Rig expression was observed in skeletal muscle, colon, thymus, spleen, kidney, liver, small intestine, placenta, lung or peripheral blood leukocytes.

In another experiment, a multiple human tissue poly(A)⁺ mRNA Northern slot blot (Clontech) was probed with a Rig ORF probe (See, FIG. 4, Panels B and C). The probe used in the Northern slot blot, and pre-hybridization/hybridization/washing was performed as described above. FIG. 4, Panel C provides the key to the RNA samples in the slot blot. As shown in FIG. 4, Panel B, strong positive signals indicative of Rig message were detected in most adult brain and cardiac structures, as well as fetal brain and heart tissue. Significantly weaker signals were also observed in additional adult tissues.

### EXAMPLE 4

### Rig RNA Expression in Tumor-Derived Neural Cell Lines

In this experiment, the expression pattern of the Rig gene was analyzed in tumor-derived neural cell lines by Northern blotting. Total RNA was isolated from cultured U251 (a human glioblastoma), TC32 (a human Ewing sarcoma), TC106 (a human PNET—primitive neuroectodermal tumor), A673 (human rhabdomyosarcoma/undifferentiated carcinoma; ATCC CRL No. 7910), SH5Y (a human neuroblastoma), and NE115 (a murine neuroblastoma) cell lines using RNeasy® total RNA isolation kits (Qiagen). Samples containing 10 μg of total RNA were resolved by formaldehyde-denaturing gel electrophoresis using a 0.8% agarose-formaldehyde gel, then transferred to Ambion® BRIGHTSTAR™ nylon membranes. These blots were probed with a random-primed ³²P-dCTP labelled full-length Rig cDNA (as described in Example 3) using Ambion® ULTRA-hyb™ hybridization reagent according to the manufacturer's instructions, followed by autoradiography.

As shown in FIG. 5, virtually no Rig mRNA was detected in the U251 and TC106 cell lines, whereas Rig expression varied in the other cell lines tested. The lower panel in FIG. 5 shows loading-normalization staining of 28S ribosomal RNA from the same blot. No genomic deletions of the Rig gene were found, as determined by Southern blot (data not shown).

### EXAMPLE 5

### Clonogenic Focus Formation Assay

In this experiment, the effect of Rig expression on cell growth was analyzed by the stable introduction of expression vectors encoding wild-type or mutant Rig proteins into NIH-3T3 cells, and subsequently observing the ability of the transfected cells to form clonogenic colonies. The expression vectors used in this experiment were based on the pZIP-NeoSV(x)1 retroviral-based expression vector (Cepko et al., Cell 37(3):1053-1062 [1984]), which carries the gene encoding neomycin resistance (neo). This vector was modified to contain a BamHI/EcoRI multiple cloning site (MCS) and an upstream hemagglutinin (HA) tag (Fiordalisi et al., Methods Enzymol., 332:3-36 [2001]) to yield the pZIP-Neo(HA) vector. Constitutive production of recombinant protein under the control of the Moloney murine leukemia virus (MoMuLV) LTR promoter results in the synthesis of HA-tagged fusion proteins. A pZIP-Neo(HA) vector encoding the Rig protein was produced by cloning a BamHI/EcoRI-compatible Rig ORF PCR fragment into those same sites in pZIP-Neo(HA). A pZIP-Neo(HA) vector encoding a mutant Rig(S17N) protein was produced using the Stratagene QuikChange™ site directed mutagenesis kit. This mutagenesis used the following primers:

| | |
|---|---|
| 5′-GCGTGGGCAAGAACTCGCTGG-3′ | (SEQ ID NO:6) |
| 5′-CCAGCGAGTTCTTGCCCACGC-3′ | (SEQ ID NO:7) |

The Rig(S21N) mutant protein is analogous to the Ras(S17N) mutation, which acts in a dominant-negative manner by abolishing GTP-binding. In the Ras protein, this mutation has been shown to result in dominant-negative inhibition of cell growth and transformation (Nature Cell Biol., 1(2):E25-E27 [1999]).

NIH-3T3 cells were grown in 6 cm dishes and transfected by the calcium phosphate (CaPO₄) precipitation method. Cells were exposed to the transfected plasmids for 4 hours followed by a 4 minute glycerol shock (7.5% v/v) at 25° C. Each plate was transfected with 100 ng plasmid DNA of one of three vectors:

(a) the empty pZIP-Neo(HA) expression vector,

(b) the pZIP-Neo(HA) vector encoding Rig(WT), or

(c) the pZIP-Neo(HA) vector encoding a Rig(S21N) mutant.

Approximately 48 hours following transfection, stable NIH-3T3 transfectants harboring the expression vectors were selected in 500 μg/ml of the neomycin-analog G418/GENTICIN® (GIBco/Life Technologies) for a minimum of two days. Surviving cells were maintained on the same plate in culture and allowed to form clonal cell colonies. After 10-14 days, cells were stained with crystal violet to facilitate observation and colony counting. This experiment was performed in duplicate at least 3 times.

Representative images of the transfected plates are shown in FIG. 6, Panel A. As indicated in the Figure, cells transfected with the empty expression vector pZIP-Neo(HA) (vector) formed abundant neo resistant colonies. However, cells transfected with wild-type Rig did not survive G418 selection. Cells transfected with the dominant-negative form of Rig also showed abundant colony formation following G418 selection. Thus, when stably expressed, Rig inhibits or prevents cell growth. Similar results were observed using COS-7 (SV-40 transformed, African Green Monkey kidney cell line; ATCC CRL No. 1651), HEK-293T (a transformed human embryonal kidney cell line, ATCC CRL No. 1573), TC32 (a human Ewing sarcoma), A673 (human rhabdomyosarcoma/undifferentiated carcinoma; ATCC CRL No. 7910), U251 (a human glioblastoma), TC106 (a human PNET—primitive neuroectodermal tumor) and SH5Y (a human neuroblastoma) cell lines (data not shown). It was for this reason that this gene and protein were called “Rig,” for Ras-related Inhibitor of Growth.

FIG. 6, Panel B, shows immunoblotting results for lysates from the NIH-3T3 cell lines containing the empty expression vector or expression vector encoding the Rig(S17N) mutant. An anti-HA primary antibody (BAbCo) and a horseradish peroxidase (HRP)-conjugated goat anti-mouse secondary antibody (Amersham) were used to develop this blot. As shown in the Figure, expression of the mutant HA-tagged Rig(S21N) protein was confirmed, demonstrating that the result of the clonogenic colony assay was not the result of deletion or misexpression of the mutant Rig protein.

### EXAMPLE 6

### Elk-1 Dependent Luciferase Assay

In this Example, experiments performed to measure the ability of Rig to regulate Ras signalling based on Elk-1 transcription factor activity are described. This experiment used a transient transfection assay to observe the ability of the Rig protein to regulate the transcriptional activity of the Elk-1 transcription factor in the presence of an activated Ras (i.e., in a Ras-dependent manner). The Elk-1 transcription factor was expressed as a fusion protein with the Gal4 DNA binding domain, and transcriptional activation activity of the fusion protein was measured by a luciferase reporter construct containing 5 tandem Gal4 DNA-binding sites (Stratagene).

A Rig(wt) expression vector was produced using the pCDNA3.1 expression vector (Invitrogen). The wild-type Rig coding sequence was generated as a PCR product with BamHI/EcoRI compatible ends, and directionally cloned into those same sites in pCDNA3. 1, to yield Rig(wt)/pCDNA3.1.

In this transient transfection assay, NIH-3T3 cells were plated in 6-well tissue culture plates and cultured to approximately 60% confluence. These cells were co-transfected using the calcium phosphate (CaPO₄) precipitation method with 125 ng of a Gal-Elk-1 expression vector (Stratagene), 50 ng of a Ras(G12V)/pCGN mammalian expression vector driven by the CMV promoter region (Fiordalisi et al., Methods Enzymol., 332:3-36 [2001]), and 250 ng of the Gal-Luciferase reporter plasmid (Stratagene). In addition, cells also received various quantities (10-1000 ng) of the Rig(wt)/pcDNA3.1 expression vector.

Following transfection, cells were cultured for 48 hours in culture medium containing 10% FBS, then shifted to culture medium containing 1% FBS overnight. The next day, the resulting quiescent transfected cells were harvested in luciferase lysis buffer and lysed for 30 minutes at 25° C. Luciferase activity was measured using a Pharmingen Luciferase Assay Kit and luminometer.

The results of this assay are shown in FIG. 7. Data are represented as percent of Ras-mediated Elk-1 activity, where the observed Elk-1 activity using only Ras without any exogenous Rig was normalized to 100%. Each data point is a mean of at least two experiments, with each data point representing duplicate transfections. The mean is expressed with the standard error, indicated by vertical lines. As seen in the Figure, Rig has an antagonistic effect on Ras-dependent Elk-1 activity even when as little as 10 ng of the Rig(wt) expression vector is introduced into cells. Increasing concentrations of the Rig(wt) expression vector resulted in more pronounced Elk-1 inhibitory effects.

### EXAMPLE 7

### Ras Transformation (Focus Formation) Assay

In this Example, experiments conducted to assess the ability of Rig to inhibit the Ras-mediated transformation of NIH-3T3 cells are described. This experiment involved the use of the focus formation assay method described in Example 6.

Identical numbers of NIH-3T3 mouse fibroblast cells were cultured in 60 cm tissue culture dishes and transfected with Ras, Rig and Rap expression vectors, as discussed below, using the calcium phosphate (CaPO₄) technique. The Rig(wt)/pCDNA3.1 vector, as described in Example 6, was used in this experiment. In addition, a pCGN expression vector encoding mutant Rap(Q63E) protein was produced by site-directed mutagenesis using an M13-Rap phage clone encoding the wild-type Rap protein as a template. The Rap mutant was identified by ³²P-labelled oligonucleotide probe differential hybridization, and confirmed by sequencing. The mutant nucleotide sequence was subcloned into the pCGN expression vector using the BamHI cloning sites. The Rap gene, and specifically the Rap(Q63E) mutant allele, has been previously shown to antagonize Ras transformation (Clark et al., J. Biol. Chem., 272:10608-10615 [1997]), and was used in this experiment as a positive control.

Each transfection contained 50 ng of a pCGN-Ras(G12V) mammalian expression vector driven by the CMV promoter region (Fiordalisi et al., Methods Enzymol., 332:3-36 [2001]), and with 500 ng of either (a) an empty pCDNA3.1 mammalian expression vector, (b) a pCDNA3.1-Rig(wt) expression vector, or (c) a pCGN-Rap(Q63E) mutant expression vector.

The cells were exposed to the DNA-transfection solution for 4 hours followed by a 4 minute glycerol shock (7.5% v/v) at 25° C. Following the glycerol shock, the medium was replaced with fresh medium of the same type every 2-3 days for 10-14 days. At the end of this time, the number of transformed cell foci was visualized using an inverted microscope and scored on each transfected plate.

The results of this experiment are shown in FIG. 8. Focus formation values are indicated as percentages of the focus formation with Ras(G12V) transformation without a co-expressed gene. Results are averages of three independent experiments, with each transfection done in triplicate. The standard error of the mean is depicted as a narrow line on each bar of the histogram.

As shown in FIG. 8, co-transfection of Ras(G12V) with Rap(Q63E) inhibited the Ras-mediated transformation. Furthermore, co-transfection of the Rig(wt) expression vector with Ras(G12V) also resulted in inhibition of Ras-induced foci formation.

### EXAMPLE 8

### In Vivo Rig/Raf-1 Co-immunoprecipitation

In this Example, experiments to assess the ability of Rig and Raf-1 to interact in vivo are described. These experiments (results shown in FIG. 9) used a standard in vivo co-transfection/co-immunoprecipitation protocol, where cultured mammalian cells were cotransfected with FLAG-tagged and/or hemagglutinin (HA)-tagged mammalian expression vector constructs, followed by immunoprecipitation with an anti-Raf-1 antibody and Western immunoblotting using either anti-FLAG or anti-HA primary detection antibodies. The assay was conducted using HEK-293T cells, a transformed human embryonal kidney cell line.

Briefly, HA-RAF-1 fusion protein expression vector was produced by generating a full-length BamHI-compatible PCR fragment from a Raf-1 template (Fabian et al., Proc. Natl. Acad.Sci. USA 91:5982-5986 [1994]), then cloned into the BamHI site of pCGN, which also encodes the HA epitope (Fiordalisi et al, Methods Enzymol., 332:3-36 [2001]). The construct was confirmed by sequencing.

A pCDNA3.1 (FLAG) expression vector was produced by inserting the FLAG epitope in-frame upstream of the BamHI restriction site of the multiple cloning region of pCDNA3.1 (Invitrogen). A FLAG-Rig expression vector was made by cloning a BamHI/EcoRI fragment containing the Rig ORF into the same sites of pCDNA-FLAG. An HA-H-Ras fusion protein was made by subcloning a BamHI fragment containing the H-Ras ORF into pCGN. An HA-K-Ras fusion gene was made by subcloning a BamHI-compatible PCR product into the pCGN vector, and confirmed by sequencing.

HEK-293T cells were cultured in 10 cm dishes and transfected with expression vector DNA using the calcium phosphate (CaPO₄) precipitation method. All cultures received 5 μg of the HA-Raf-1 expression vector. In addition, the cells were also co-transfected with either (a) 5 μg of the FLAG-Rig expression vector, (b) 5 μg of the HA-H-Ras expression vector, or (c) 5 μg of the HA-K-Ras expression vector.

Following transfection, the cells were cultured in DMEM containing 1% FBS for 48 hours. After this time, RIPA lysis buffer (50 mM HEPES, pH 7.5, 150 mM NaCl, 1% NP-40, 0.5% sodium deoxycholate, 1 mM Na₃VO₄, 50 mM NaF, 1 mM phenylmethylsulfonyl fluoride [PMSF], 5 mM benzamidine, 0.1% aprotinin, 1 μg/ml leupeptin) was added to the plates, and the cells were scraped into eppendorf tubes on ice. The resulting lysate was clarified by centrifugation at 13,000×g. HA-Raf-1 was immunoprecipitated from lysates by incubation for 1-2 hours with a rabbit polyclonal anti-Raf-1 antibody (Santa Cruz Biotechnology; Cat. No. C-12). Immune complexes were immobilized with A/G Plus-agarose beads (Santa Cruz Biotechnology), washed in RIPA buffer, resuspended in 2× sample buffer and resolved by 4-20% gradient SDS-PAGE, then blotted onto a polyvinylidene difluoride (PVDF) membrane. Blots were incubated for one hour in blocking buffer (Tris-buffered saline [TBS] containing 5% (w/v) nonfat dry milk and 0.1% Tween-20), then incubated for one hour with a polyclonal anti-FLAG antibody (Sigma; M2 antibody) or anti-HA antibody (BAbCo) at a 1:5000 dilution in blocking buffer. Membranes were washed 3 times in washing buffer (TBS containing 0.1% Tween-20) for 10 minutes each wash, then incubated for 30 minutes with horseradish peroxidase-conjugated goat anti-rabbit IgG (Amersham) diluted 1:5000 in blocking buffer, then washed 4 times in washing buffer for 10 minutes each wash. Membranes were then incubated in enhanced chemiluminescent substrate (ECL; Amersham) and the signal was detected on X-ray film.

The corresponding Western blots are shown in FIG. 9. The top panel of this Figure shows a Western blot probed with an anti-FLAG primary antibody, while the lower panel shows the same blot stripped and reprobed with an anti-HA primary antibody. The expression vectors used in each transfection are indicated at the bottom. As indicated in the Figure, immunoprecipitation with the anti-Raf-1 antibody coprecipitated the recombinant HA-H-Ras, HA-K-Ras and FLAG-Rig proteins, indicating that these proteins are associated with Raf-l in HEL-293T cells.

### EXAMPLE 9

### Generation of Polyclonal Anti-Rig Antiserum

A polypeptide from the carboxyl-terminus of the Rig protein was used to produce anti-Rig polyclonal antiserum. Specifically, a 20 amino acid synthetic peptide corresponded to amino acids 175 to 194 of the human Rig protein, having the sequence:

L¹⁷⁵NIDGKRSGKQKRTDRVKGK (SEQ ID NO:8) was used to immunogenize 3-9 month old New Zealand white rabbits and immune serum was subsequently collected using standard techniques. This particular peptide was chosen based upon the uniqueness of its amino acid sequence as determined by database query.

Two rabbits were immunized with this peptide using standard techniques for antibody production. The Rig peptide was conjugated to keyhole limpet hemocyanin (KLH) and emulsified by mixing with an equal volume of Freund's Adjuvant. The amount of antigen per immunization was 0.1 mg, injected into three subcutaneous dorsal sites. The animals received boosts at 2, 6 and 8 weeks. At the time of collections, animals were bled from the auricular artery, blood was allowed to coagulate, and serum was collected by centrifugation. Bleeds were obtained at weeks 4, 8, and 10, and tested for the presence of anti-Rig antibody. The serum was stored at −20° C. until needed. The antiserum obtained from the week 10 bleed was used in the experiments described herein.

The antibody titer was determined with an enzyme linked immunosorbent assay (ELISA) with peptide (i.e., SEQ ID NO:8) bound in solid phase at a concentration of 1 μg peptide/well. Detection was accomplished using biotinylated anti-rabbit IgG, horseradish peroxidase-strepavidin (HRP-SA) conjugate, and ABTS substrate/hydrogen peroxide solution (KPL). Absorbance was measured at OD₄₀₆ₙₘ. Only one of the two rabbits immunized produced Rig-reactive antibody. The anti-Rig antibody showed no cross-reactivity with any of the other monomeric GTPase GST fusion proteins tested, including Rap, Ras, Rheb and RRP22 (data not shown).

### EXAMPLE 10

### Cellular Growth Rate in Rig Overexpressing Tumor Cell Lines

In this Example, experiments to assess the ability of Rig expression to regulate the growth of neural tumor cell lines are described. In these experiments the growth rate was monitored by enumerating cells following the induced expression of Rig within the U251 glioblastoma and A673 rhabdomyosarcoma tumor cell lines. Due to the inhibitory effect of Rig expression on cell growth and survival, Rig expression in this experiment was controlled through the use of a recombinant tetracycline-inducible Moloney murine leukemia virus (M-MuLV)-based retroviral vector, pLRT.

The LRT retroviral vector contains retroviral sequences that permit the production of non-replicating, infective virions, in the presence of helper functions provided by a suitable packaging cell line. The LRT vector contains a cloning site and regulatory sequences that facilitate the subcloning and conditional expression of a desired ORF, which is under the transcriptional control of a tetracycline-sensitive promoter, such that addition of tetracycline (or the tetracycline-analog doxycycline) results in induced transcription and protein expression. In addition, the LRT virus encodes and constitutively expresses the blasticidin-S-deaminase (BSD) gene product, which imparts resistance to the cytotoxic drug blasticidin to cells which harbor the virus.

For safety purposes, variants of the U251 and A673 neural tumor-derived human cell lines were constructed that express the ecotropic docking receptor (Wang et al., Nature 352:729-731 [1991]). To accomplish this, the gene encoding the ecotropic docking receptor was subcloned into the pBabe retroviral vector (Morgenstern et al., Nucl. Acids Res., 18:3587-3596 [1990]), then transfected into the amphotropic packaging cell line PA317 (Kwok et al., Proc. Natl. Acad. Sci. USA 83:4552-4555 [1986]), followed by selection of stable transfectants in puromycin. Retroviral-conditioned medium produced by the engineered PA317 cells was used to infect human U251 and A673 neural cells, followed by selection for stably infected recipient cells in puromycin. Thus, these engineered U251 and A673 cells are now susceptible to infection by virion produced by an ecotropic retroviral producer cell line.

To construct the Rig-LRT vector, an HindIII/EcoRI fragment containing the Rig ORF and the FLAG epitope tag was excised from the Rig-pCDNA3.1-FLAG construct (previously described), and cloned into the HindIII/EcoRI sites of pBluescript® (Stratagene). From the modified pBluescript® vector, an XhoI/NotI fragment was excised and cloned into the same sites in the pLRT plasmid, to form pLRT-Rig-FLAG. This pLRT-Rig-FLAG vector was then transfected into the Phoenix ecotropic retroviral packaging cell line (Nolan and Shatzman, Curr. Opin. Biotechnol., 9(5):447-450 [1998]; Hofmann et al., Proc. Natl. Acad. Sci. USA 93(11):5185-5190 [1996]). Infective pLRT-Rig-FLAG virions were collected from the Phoenix culture supernatants, and these supernatants were then used to stably infect the engineered ecotropic receptor-positive U251 and A673 host cells. Following infection, stable integration of the pLRT-Rig-FLAG viral genome was selected by the addition of 5 μg/ml of blasticidin, and polyclonal U251(pLRT-Rig-FLAG) and A673(pLRT-Rig-FLAG) cell lines were subsequently established.

Following establishment of the stably infected cell lines, parallel cultures of approximately 4×10⁴ cells of the U251(pLRT-Rig-FLAG) and A673(pLRT-Rig-FLAG) lines in 6 cm dishes were established under 1% and 5% (i.e., reduced) serum conditions, respectively. After 24 hours, one set of cultures was supplemented with 2 μg/ml doxycycline. At various time points over a period of 6 days, growth rates of the cultures were determined by trypsinizing, harvesting, and counting cells with a Coulter Counter.

The resulting growth curves are shown in FIG. 10, Panel A, with the growth curves of the U251(pLRT-Rig-FLAG) cells on the left, and the growth curves of the A673(pLRT-Rig-FLAG) cells on the right. Cultures treated with doxycycline are shown with a solid square, while untreated cultures are shown with an open square. Each experimental condition represents the mean (± standard error of the mean) of three experiments performed in duplicate or triplicate. As indicated in this Figure, both the U251 and A673 cell lines showed similar responses (i.e., addition of the doxycycline resulted in the inhibition of cell growth compared to untreated cells). Results from these experiments indicate an indirect correlation between Rig expression and growth rate in neural tumor-derived cell lines.

In order to confirm doxycycline-induced Rig-FLAG expression, the counted cells were pelleted by centrifugation and lysed in RIPA buffer. The resulting cell extracts from doxycycline-treated and untreated cells at 48 and 96 hours following drug treatment were analyzed by Western blot using the anti-Rig antiserum (as described in Example 9) as the primary antibody, and a goat anti-rabbit HRP-conjugated antibody (Amersham) as the secondary antibody, according to the same protocol as described in Example 11. This blot is shown in FIG. 10, Panel B. As indicated in this Figure, Rig-FLAG expression is strongly induced at 48 and 96 hours following drug treatment, but non-treated cells showed no Rig-FLAG expression at these same time points. Identical results were obtained in a another experiment using an anti-FLAG antibody as the primary antibody (data not shown). In both the U251(pLRT-Rig-FLAG) and A673(pLRT-Rig-FLAG) cells, the induced Rig-FLAG expression was seen as early as 24 hours post-drug treatment and was sustained through 5 days post-treatment, whereas expression was not detectable in uninduced cells (data not shown). Thus, Rig expression was rapidly upregulated by the addition of doxycycline, with no apparent background expression.

### EXAMPLE 11

### Rig Western Immunoblotting in Normal and Tumor Tissues, in Human and Mouse Specimens

In this Example, experiments to assess endogenous human Rig protein expression in normal tissue and a number of neural tumorigenic samples using a Western immunoblotting technique and the anti-Rig polyclonal antiserum of Example 9 are described. These experiments also tested the ability of the polyclonal anti-human Rig antiserum to detect Rig protein in mouse tissue.

In one experiment, protein lysates were prepared from human neural tissue (normal striatum), oligodendroglioma (05 and 011) and end-stage astrocytoma (U15, U42 and U49) with a dounce-homogenizer in chilled buffer. Lysates were clarified, and equivalent amounts of total protein from the lysate samples were resolved by SDS-PAGE, and transferred to PVDF membrane (Immobilon-P membrane; Millipore). Western immunoblotting analysis employed the anti-Rig antiserum of Example 9 as the primary antibody at a dilution of 1:1,000 and goat anti-rabbit HRP conjugate as the secondary antibody at a dilution of 1:5,000. Prior to probing, blots were incubated for one hour in blocking buffer (Tris-buffered saline [TBS] containing 5% (w/v) nonfat dry milk and 0.1% Tween-20), then incubated for one hour with the primary antibody. Membranes were washed 3 times in washing buffer (TBS containing 0.1% Tween-20) for 10 minutes each wash, then incubated for 30 minutes with the secondary antibody, and then washed 4 times in washing buffer for 10 minutes each wash. Membranes were then incubated in enhanced chemiluminescent substrate (ECL; Amersham) and the signal was detected on X-ray film. As a control, this same immunoblot was stripped and reprobed using an anti-β-tubulin primary antibody (Santa Cruz Biochemicals) and goat anti-rabbit secondary antibody (Amersham).

These two blots are shown in FIG. 11. As indicated in the lower blot, Rig expression is observed in the normal striatum tissue. However, Rig expression is greatly diminished or absent in 3 of 5 of the tumor tissues tested. Detection of the β-tubulin control protein confirmed that protein samples from the tumor specimens were not degraded or underloaded.

In another experiment using this same immunoblotting protocol, Rig expression was examined in a series human astrocytoma tumor tissue samples. The tumor samples used in this experiment were placed in two clinical groups, low-grade tumors (13 samples) and high-grade tumors (6 samples), where the low-grade tumors were determined to be less advanced, and patients with these tumors had a more favorable prognosis than had patients with high-grade tumors. Protein lysates were prepared from normal human neural tissue (normal striatum) and from astrocytoma tumor tissues, using the same protocol described above. Equivalent amounts of total protein from the tumor tissue lysate samples were resolved by SDS-PAGE, and transferred to a PVDF membrane (Immobilon-P membrane; Millipore). The same Western blotting protocol described above, was used with the anti-Rig antiserum as the primary antibody, and goat anti-rabbit-HRP as the secondary antibody. Samples of normal human striatum tissue (labelled N) were also run in parallel.

These immunoblots are shown in FIG. 12. As indicated in this Figure, a protein species which cross-reacts with the anti-Rig antiserum is observed in the human normal striatum tissue. In the low grade astrocytoma tumor samples, that protein is absent or reduced in 8 of 13 tumor specimens. More strikingly, the anti-Rig polypeptide is completely absent in 5 of 6 high grade astrocytoma specimens, and greatly reduced in the remaining high grade specimen. Thus, based on the experiments shown in FIGS. 11 and 12, Rig polypeptide expression is inversely correlated with tumorigenicity.

Another experiment was conducted using this same immunoblotting protocol to determine whether the anti-Rig antiserum produced in response to a human Rig peptide (See, Example 9), reacts with the murine Rig protein. Protein lysates were prepared from human neural tissue (normal striatum) and mouse brain, as described above. Equivalent amounts of total protein from the human and mouse tissue lysate samples were resolved by SDS-PAGE, and transferred to a PVDF membrane (Immobilon-P membrane; Millipore). The Western blot protocol described above was used, with anti-Rig antiserum used as the primary antibody, and goat anti-rabbit-HRP used as the secondary antibody.

This immunoblot is shown in FIG. 13. Molecular weight markers are in the center lane. As indicated in the Figure, a protein species of approximately 30 kDa detected by the anti-human Rig antiserum was observed in both the human normal striatum and the mouse brain tissue. Thus, the antiserum raised against the human Rig peptide appears to detect the mouse Rig protein, as well as the human Rig protein. Also as indicated previously, a human Rig nucleic acid probe detected a transcript in a total RNA sample from the mouse NE115 neuroblastoma cell line (See, FIG. 5).

### EXAMPLE 12

### In Vitro Prenylation Assay

In this Example, experiments performed to determine the in vitro prenylation characteristics of Rig, Ras and Rap are described. Radiolabelled farnesyl pyrophosphate, radiolabelled geranylgeranyl pyrophosphate or radiolabelled mevalonate were alternatively used as substrates in these assays, to determine the prenylation specificity of each GTPase protein. In addition, the ability of a farnesyl transferase inhibitor (FTI; namely, FTI-277, Calbiochem, Catalog No. 344555) to inhibit the prenylation of Rig, Ras and Rap was examined. Purified GST-Rig, GST-H-Ras (G12V) and GST-Rap fusion proteins were used in the in vitro prenylation experiments. The H-Ras(G12V) protein was included in the prenylation assays to serve as a positive control (Clark et al., Proc. Natl. Acad. Sci. USA 90:4887-4891 [1993]).

The GST-Rig fusion construct was made by cloning a restriction fragment containing the Rig ORF in frame into the BamHI/EcoRI sites of pGEX-2T (Pharmacia). The vector encoding GST-H-Ras(G12V) was made by subcloning an H-Ras (G12V) BamHI/EcoRI PCR product into the BamHI/EcoRI sites of the pGEX-2T vector. The product was confirmed by sequencing. The GST-Rap fusion construct was produced as described by Clark et al. (Clark et al., J. Biol. Chem., 272:10608-10615 [1997]).

The resulting plasmids were transformed into competent XL1-Blue Escherichia coli (Stratagene), and the GST-fusion proteins produced by the bacteria were isolated by standard techniques, according to the manufacturer's protocol (Pharmacia), with minor modification (inclusion of 100 μM GTP and the omission of EDTA from the protein collection/purification buffers). Briefly, bacteria were grown to an OD₆₀₀ of 0.1 in LB broth. At that time, 100 mM isopropyl-β-thiogalactopyranoside (IPTG) was added to the culture. Four hours following IPTG induction, the bacteria were pelleted at 3,000 RPM for 20 minutes, and resuspended in 5 mL lysis buffer (phosphate buffered saline [PBS] containing 100 μM GTP, 0.1% Triton X−100, leupeptin and aprotinin). The lysate was sonicated, then clarified by centrifugation at 10,000 RPM for 30 minutes. The resulting lysate was used as starting material for glutathione-conjugated agarose bead purification (Sigma) using standard techniques. Following purification to near homogeneity, concentrations of the GST fusion proteins were estimated using SDS-PAGE followed by Coomassie Blue staining.

Each in vitro prenylation reaction contained approximately 1-2 μg alternatively of GST, GST-Rig, GST-H-Ras(G12V) or GST-Rap. Each reaction mixture also contained rabbit reticulocyte lysate (Promega, Catalog No. L4151) containing either 2.5 μCi [³H]-mevalonate (mev), 2.5 μCi [³H]-farnesyl pyrophosphate (FPP), or 2.5 μCi [³H]-geranylgeranyl pyrophosphate (GGPP). Reactions were incubated for one hour at 30° C. Affinity complexes were isolated by centrifugation, suspended in 1×SDS-PAGE sample loading buffer containing 5% β-mercaptoethanol, boiled, and resolved by SDS-PAGE. The gel was soaked in fixing buffer (water with 10% methanol and 10% acetic acid), soaked in autofluor (National Diagnostics, Catalog No. LS315) and analyzed by autoradiography.

As shown in FIG. 14, Panel A, mevalonate (mev) was a suitable substrate for the prenylation of GST-Rig, GST-Ras(G12V) and GST-Rap. Farnesyl pyrophosphate (FPP) was a suitable substrate for the prenylation of GST-Rig and GST-Ras(G12V), and geranylgeranyl pyrophosphate (GGPP) was a suitable substrate for the prenylation of Rig and Rap. Also as shown in FIG. 14, Panel A, GST protein was not prenylated by any of the three prenylation substrates. The bottom image of FIG. 14, Panel A, shows a Coomassie blue-stained polyacrylamide gel of the proteins used in the prenylation reactions, confirming that each protein was accurately synthesized, undegraded, and used in approximately the same concentration in each reaction. Thus, Rig undergoes post-translational prenylation, being modified by farnesyl and geranylgeranyl isoprenoid groups under these in vitro conditions.

In a second experiment, the sensitivity of mevalonate prenylation of GST-Rig, GST-Ras and GST-Rap, to increasing amounts of farnesyl transferase inhibitor (FTI-277; Calbiochem) was assessed. The same methodology as describe above was used in this experiment. The in vitro prenylation assays contained alternatively equal amounts of GST-Rig, GST-Ras(G12V) or GST-Rap. Each reaction mixture contained rabbit reticulocyte lysate, 2.5 μCi [³H]-mevalonate and increasing concentrations of FTI ranging from 1-100 nM. Reactions were incubated for one hour at 30° C., then stopped by the addition of SDS-PAGE sample buffer. The samples were boiled and resolved by reducing SDS-PAGE. The gel was fixed, soaked in autofluor and analyzed by autoradiography. As shown in FIG. 14, Panel B, prenylation (i.e., radiolabelling) of GST-Rig and GST-Ras was sensitive to FTI-277. Of the two proteins, GST-Ras showed a greater sensitivity, although farnesylation of both proteins was completely abolished at 100 nM FTI concentration. GST-Rap farnesylation by mevalonate did not show sensitivity to FTI in the 1-100 nM FTI concentration range.

### EXAMPLE 13

### Rig Subcellular Localization

In this experiment, the subcellular localization of the Rig protein was analyzed. To accomplish this, a green fluorescent protein (GFP) fusion was constructed to yield a GFP-Rig chimeric indicator protein. Following transient expression of this protein in a host cell, the protein was visualized and photographed. An ecdysone-inducible, two part mammalian conditional expression system was used in this experiment to conditionally express the GFP-Rig fusion protein, according to the manufacturer's protocols (Invitrogen).

A vector comprising the Rig and GFP coding sequences, operably joined in-frame, was engineered using the two-component pIND(SP11) inducible expression vector system (Invitrogen). In this system, the ORF of interest is placed under the transcriptional control of the ecdysone promoter, and the ecdysone receptor is constitutively expressed from a second plasmid. To make the inducible GFP-Rig construct, pIND(SP1) was modified by cloning the GFP open reading frame within the NheI/HindIII restriction sites of the multiple cloning cassette, yielding GFP/pIND(SP1). Subsequently, the Rig open reading frame (along with the FLAG epitope) was excised as a HindIII/NotI restriction fragment obtained from the pCDNA3-FLAG/Rig(WT) construct, and cloned into GFP/pIND(SP1), to form GFP-Rig/pIND (SP1). Expression of the Rig coding sequence is inducible by the addition of the ecdysone-analog molecule Ponasterone A to 5 μM, in a mammalian cell stably cotransfected with the pVgRXR plasmid (Invitrogen), which constitutively expresses the heterodimeric ecdysone receptor.

The ecdysone-inducible vector encoding the GFP-Rig fusion protein was transiently transfected into a human embryonal kidney (HEK)-293-based cell line (E293) which constitutively expresses the ecdysone receptor subunits from the pVgRXR plasmid (Invitrogen). The transfected cells were exposed to ponasterone A 48 hours following transfection. The cells were viewed at 24 hours post-ponasterone A induction and GFP fluorescence was visualized on an inverted fluorescence microscope with a 40× objective under excitation/emission wavelengths of 488 nm and 507 nm, respectively, A parallel control experiment was conducted using an ecdysone-inducible expression vector containing the GFP open reading frame fused to a nuclear localization signal (NLS).

These fluorescence microscopy images are shown in FIG. 15. Cells that express the GFP-NLS fusion are shown in the left panel, while the right panel shows an image of cells expressing the GFP-Rig chimera. The cells expressing the GFP-NLS species show largely nuclear fluorescence, while the GFP-Rig species clearly localizes to the plasma membrane. Thus, like Ras proteins, Rig also appears to localize to the plasma membrane.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, genetics, cancer biology or related fields are intended to be within the scope of the following claims.

What is claimed is:

1. A recombinant expression vector comprising a nucleotide sequence encoding the polypeptide set forth in SEQ ID NO:5.

2. The recombinant expression vector of claim 1, wherein said nucleotide sequence is the nucleotide sequence set forth in SEQ ID NO:4.

3. The recombinant expression vector of claim 1, wherein said vector further comprises a replication-defective virus.

4. A host cell comprising the recombinant expression vector of claim 1, wherein said host cell is selected from the group consisting of prokaryotic host cells and eukaryotic host cells.

5. An antibody directed against at least a portion of the amino acid sequence of SEQ ID NO:5, wherein said antibody is selected from the group consisting of monoclonal antibodies and polyclonal antibodies.

6. A method for detecting nucleic acids encoding Rig in a sample, comprising the steps of:
a) providing:
i) a sample encoding Rig,
ii) a probe comprising nucleic acid having complementarity to at least a portion of the nucleotide sequence of SEQ ID NO:4,
b) combining said sample and said probe under conditions wherein a hybridization complex is formed between said probe and said nucleic acid in said sample, and
c) detecting said hybridization complex.

7. The method of claim 6, wherein said sample is selected from the group consisting of total cellular RNA, polyA RNA and genomic DNA.

8. The method of claim 6, wherein said sample comprises tumor tissue.

9. The method of claim 6, wherein said sample is from a human subject.

10. The method of claim 6, wherein said method comprises a Northern blotting protocol.

11. A method for amplifying nucleic acids encoding Rig in a sample, comprising:
a) providing:
i) a sample comprising nucleic acids encoding Rig,
ii) a DNA polymerase;
iii) two oligonucleotides having complementarity to the nucleotide sequence of SEQ ID NO:4; and
iv) PCR amplification reagents;
b) combining said sample, said DNA polymerase, said oligonucleotides, and said PCR amplification reagents,
c) annealing said oligonucleotides to said nucleic acid in said sample;
d) extending said oligonucleotides with reiterated DNA synthesis under conditions such that said nucleic acid is amplified to produce an amplified product; and
e) detecting said amplified product.

12. The method of claim 11, wherein said DNA polymerase has both DNA-dependent DNA polymerase activity and reverse transcriptase RNA-dependent DNA polymerase activity.

13. The method of claim 11, wherein said sample is from a human subject.

14. The method of claim 11, wherein said sample comprises tumor tissue.

15. The method of claim 11, wherein said nucleic acid is selected from DNA and RNA.

16. The method of claim 11, wherein said two oligonucleotides comprise SEQ ID NO:2 and SEQ ID NO:3.

17. A method for detecting Rig polypeptide in a sample, comprising:
a) providing:
i) a sample comprising Rig, and
ii) an antibody of claim 5;
b) contacting said sample with said antibody under conditions such that said antibody specifically binds to said polypeptide in said sample to form an antigen-antibody complex; and
c) detecting said polypeptide-antibody complex.

18. The method of claim 17, wherein said sample comprises tumor tissue.

19. The method of claim 17, wherein said sample is from a human subject.

20. The method of claim 17, wherein said method is selected from the group consisting of Western blotting, enzyme linked immunosorbent assays, immunofluorescence assays, radioimmuno assays, and immunohistochemistry.

21. A method for inhibiting cell growth, comprising the steps of:
a) providing:
i) a cell,
ii) the recombinant expression vector of claim 1, and
iii) a means for delivery of said recombinant expression vector into said cell;
b) delivering said recombinant expression vector into said cell using said means for delivery, and
c) expressing said polypeptide within said cell.

22. The method of claim 21, wherein said cell is a human cell.

23. The method of claim 22, wherein said human cell is within a human subject.

24. The method of claim 21, wherein said cell is a tumor cell.

25. The method of claim 24, wherein said tumor cell is of neural origin, and wherein said tumor cell of neural origin is selected from the group consisting of astrocytoma cells, glioblastoma cells, human Ewing sarcoma cells, primitive neuroectodermal tumor cells, rhabdomyosarcoma cells, undifferentiated carcinoma cells, and neuroblastoma cells.

26. The method of claim 21, wherein said means of delivery is selected from the group consisting of direct nucleic acid administration, liposomes, a recombinant virus, and any combination thereof.

27. The method of claim 26, wherein said recombinant virus comprises operably linked recombinant nucleotide sequences comprising a suitable promoter sequence and viral sequences, wherein said viral sequences are selected from the group consisting of adenovirus sequences, adeno-associated virus sequences, retrovirus sequences, herpes virus sequences, vaccinia virus sequences and Moloney virus sequences.

28. The method of claim 26, wherein said means of delivery is selected from local delivery and systemic delivery, and wherein local delivery is selected from the group comprising surgical delivery, implantation, and injection.
